(19) United States
(12) Patent Application Publication
(10) Pub. No.: US 2009/0143392 A1
(43) Pub. Date: Jun. 4, 2009

(54) Methods of Treating Obesity and Metabolic Disorders

(75) Inventors: Norbert Hofgen, Ottendorf-Okrilla (DE); Hans Stange, Riesa (DE); Barbara Langen, Radebeul (DE); Ute Egerland, Radebeul (DE); Rudolf Schindler, Dresden (DE); Antje Gasparic, Coswig (DE); Chris Rundfeldt, Coswig (DE); Thomas Pfeifer, Radebeul (DE)

Correspondence Address: FISH & RICHARDSON P.C., P.O BOX 1022, MINNEAPOLIS, MN, 55440-1022, US

(73) Assignee: ELBION GMBH, Radebeul (DE)

(21) Appl. No.: 12/323,188

(22) Filed: Nov. 25, 2008

Related U.S. Application Data
(60) Provisional application No. 61/004,883, filed on Nov. 30, 2007.

(51) Int. Cl. A61K 31/4985; A61P 3/00; A61P 3/04; A61P 3/10

(52) U.S. Cl. 514/250

(57) ABSTRACT

The invention relates to methods of treating or preventing obesity, type 2 diabetes, metabolic syndrome, or glucose intolerance using pyrido[3,2-e]pyrazines which are inhibitors of PDE10. The invention further relates to methods of reducing body fat or body weight.

### CROSS REFERENCE TO RELATED APPLICATIONS

This application claims the benefit of U.S. Provisional Patent Application Ser. No. 61/004,883, filed Nov. 30, 2007, the disclosure of which is incorporated herein by reference in its entirety.

### FIELD OF THE INVENTION

The invention relates to methods of treating or preventing obesity, type 2 diabetes, metabolic syndrome, or glucose intolerance using pyrido[3,2-e]pyrazines which are inhibitors of PDE10. The invention further relates to methods of reducing body fat or body weight.

### BACKGROUND

Psychotic disorders, especially schizophrenia, are severe mental disorders which extremely impair daily life. The symptoms of psychosis may be divided into two fractions. In the acute phase, it is predominated by hallucinations and delusions being called the positive symptoms. When the agitated phase abates the so called negative symptoms become obvious. They include cognitive deficits, social phobia, reduced vigilance, indifference and deficits in verbal learning and memory, verbal fluency and motor function.

Although several antipsychotics are available since, the present therapy of psychosis is not satisfactory. The classic antipsychotics, such as haloperidol, with a high affinity to dopamine D2 receptor show extreme side effects, such as extrapyramidal symptoms (=EPS) and do not improve the negative symptoms of schizophrenia so that they do not enable the patient to return to everyday life.

Clozapine which has emerged as a benchmark therapeutic ameliorating positive, negative and cognitive symptoms of schizophrenia and devoid of EPS shows agranulocytosis as a major, potential lethal side-effect (Capuano et al., Curr Med Chem 9: 521-548, 2002). Besides, there is still a high amount of therapy resistant cases (Lindenmayer et al., J Clin Psychiatry 63: 931-935, 2002).

In conclusion, there is still a need for developing new antipsychotics which ameliorate positive, negative and cognitive symptoms of psychosis and have a better side effect profile.

The exact pathomechanism of psychosis is not yet known. A dysfunction of several neurotransmitter systems has been shown. The two major neurotransmitter systems that are involved are the dopaminergic and the glutamatergic system:

Thus, acute psychotic symptoms may be stimulated by dopaminergic drugs (Capuano et al., Curr Med Chem 9: 521-548, 2002) and classical antipsychotics, like haloperidol, have a high affinity to the dopamine D2 receptor (Nyberg et al., Psychopharmacology 162: 37-41, 2002). Animal models based on a hyperactivity of the dopaminergic neurotransmitter system (amphetamine hyperactivity, apomorphine climbing) are used to mimic the positive symptoms of schizophrenia.

Additionally there is growing evidence that the glutamatergic neurotransmitter system plays an important role in the development of schizophrenia (Millan, Prog Neurobiol 70: 83-244, 2005). Thus, NMDA antagonists like phencyclidine and ketamine are able to stimulate schizophrenic symptoms in humans and rodents (Abi-Saab et al., Pharmacopsychiatry 31 Suppl 2: 104-109, 1998; Lahti et al., Neuropsychopharmacology 25: 455-467, 2001). Acute administration of phencyclidine and MK-801 induce hyperactivity, stereotypies and ataxia in rats mimicking psychotic symptoms. Moreover, in contrast to the dopaminergic models the animal models of psychosis based on NMDA antagonists do not only mimic the positive symptoms but also the negative and cognitive symptoms of psychosis (Abi-Saab et al., Pharmacopsychiatry 31 Suppl 2: 104-109, 1998; Jentsch and Roth, Neuropsychopharmacology 20: 201-225, 1999). Thus, NMDA antagonists, additionally induce cognitive deficits and social interaction deficits.

Eleven families of phosphodiesterases have been identified in mammals so far (Essayan, J Allergy Clin Immunol 108: 671-680, 2001). The role of PDEs in the cell signal cascade is to inactivate the cyclic nucleotides cAMP and/or cGMP (Soderling and Beavo, Proc Natl Acad USA 96(12):7071-7076, 2000). Since cAMP and cGMP are important second messenger in the signal cascade of G-protein-coupled receptors PDEs are involved in a broad range of physiological mechanisms playing a role in the homeostasis of the organism.

The PDE families differ in their substrate specificity for the cyclic nucleotides, their mechanism of regulation and their sensitivity to inhibitors. Moreover, they are differentially localized in the organism, among the cells of an organ and even within the cells. These differences lead to a differentiated involvement of the PDE families in the various physiological functions.

PDE10 (PDE10A) is primarily expressed in the brain and here in the nucleus accumbens and the caudate putamen. Areas with moderate expression are the thalamus, hippocampus, frontal cortex and olfactory tubercle (Menniti et al., William Harvey Research Conference, Porto, Dec. 6^{th}-8^{th}, 2001). All these brain areas are described to participate in the pathomechanism of schizophrenia (Lapiz et al., Neurosci Behav Physiol 33: 13-29, 2003) so that the location of the enzyme indicates a predominate role in the pathomechanism of psychosis.

In the striatum PDE10A is predominately found in the medium spiny neurons and they are primarily associated to the postsynaptic membranes of these neurons (Xie et al., Neuroscience 139: 597-607, 2006). By this location PDE10A may have an important influence on the signal cascade induced by dopaminergic and glutamatergic input on the medium spiny neurons two neurotransmitter systems playing a predominate role in the pathomechanism of psychosis.

Phosphodiesterase (PDE) 10A, in particular, hydrolyses both cAMP and cGMP having a higher affinity for cAMP (Kₘ=0.05 μM) than for cGMP (Kₘ=3 μM) (Soderling et al., Curr. Opin. Cell Biol 12: 174-179, 1999).

Psychotic patients have been shown to have a dysfunction of cGMP and cAMP levels and its downstream substrates (Kaiya, Prostaglandins Leukot Essent Fatty Acids 46: 33-38, 1992; Muly, Psychopharmacol Bull 36: 92-105, 2002; Garver et al., Life Sci 31: 1987-1992, 1982). Additionally, haloperidol treatment has been associated with increased cAMP and cGMP levels in rats and patients, respectively (Leveque et al., J Neurosci 20: 4011-4020, 2000; Gattaz et al., Biol Psychiatry 19: 1229-1235, 1984). As PDE10A hydrolyses both cAMP and cGMP (Kotera et al., Biochem Biophys Res Commun 261: 551-557, 1999), an inhibition of PDE10A would also induce an increase of cAMP and cGMP and thereby have a similar effect on cyclic nucleotide levels as haloperidol.

The antipsychotic potential of PDE10A inhibitors is further supported by studies of Kostowski et al. (Pharmacol Biochem Behav 5: 15-17, 1976) who showed that papaverine, a moderate selective PDE10A inhibitor, reduces apomorphine-induced stereotypies in rats, an animal model of psychosis, and increases haloperidol-induced catalepsy in rats while concurrently reducing dopamine concentration in rat brain, activities that are also seen with classical antipsychotics. This is further supported by a patent application establishing papaverine as a PDE10A inhibitor for the treatment of psychosis (US Patent Application No. 2003/0032579).

In addition to classical antipsychotics which mainly ameliorate the positive symptoms of psychosis, PDE10A also bears the potential to improve the negative and cognitive symptoms of psychosis.

Focusing on the dopaminergic input on the medium spiny neurons, PDE10A inhibitors by up-regulating cAMP and cGMP levels act as D1 agonists and D2 antagonists because the activation of Gs-protein coupled dopamine D1 receptor increases intracellular cAMP, whereas the activation of the Gi-protein coupled dopamine D2 receptor decreases intracellular cAMP levels through inhibition of adenylyl cyclase activity (Mutschler et al., Mutschler Arzneimittelwirkungen. 8^{th} ed. Stuttgart: Wissenschaftliche Verlagsgesellschaft mbH, 2001).

Elevated intracellular cAMP levels mediated by D1 receptor signalling seems to modulate a series of neuronal processes responsible for working memory in the prefrontal cortex (Sawaguchi, Parkinsonism Relat Disord 7: 9-19, 2000), and it is reported that D1 receptor activation may improve working memory deficits in schizophrenic patients (Castner et al., Science 287: 2020-2022, 2000). Thus, it seems likely that a further enhancement of this pathway might also improve the cognitive symptoms of schizophrenia.

Further indication of an effect of PDE10A inhibition on negative symptoms of psychosis was given by Rodefer et al. (Eur. J Neurosci 21: 1070-1076, 2005) who could show that papaverine reverses attentional set-shifting deficits induced by subchronic administration of phencyclidine, an NMDA antagonist, in rats. Attentional deficits including an impairment of shifting attention to novel stimuli belongs to the negative symptoms of schizophrenia. In the study the attentional deficits were induced by administering phencyclidine for 7 days followed by a washout period. The PDE10A inhibitor papaverine was able to reverse the enduring deficits induced by the subchronic treatment.

The synthesis of imidazo[1,5-a]pyrido[3,2-e]pyrazinones and some medical uses are well described in patents and the literature.

The documents EP 0 400 583 and U.S. Pat. No. 5,055,465 from Berlex Laboratories, Inc. report a group of imidazoquinoxalinones, their aza analogs and a process for their preparation. These compounds have been found to have inodilatory, vasodilatory and yenodilatory effects. The therapeutic activity is based on the inhibition of phosphodiesterase 3 (PDE3).

EP 0 736 532 reports pyrido[3,2-e]pyrazinones and a process for their preparation. These compounds are described to have anti-asthmatic and anti-allergic properties. Examples of this invention are inhibitors of PDE4 and PDE5.

WO 00/43392 reports the use of imidazo[l,5-a]pyrido[3,2-e]pyrazinones which are inhibitors of PDE3 and PDE5 for the therapy of erectile dysfunction, heart failure, pulmonic hypertonia and vascular diseases which are accompanied by insufficient blood supply.

Another group of pyrido[3,2-e]pyrazinones, reported in WO 01/68097 are inhibitors of PDE5 and can be used for the treatment of erectile dysfunction.

Further methods for the preparation of imidazo[l,5-a]pyrido[3,2-e]pyrazinones are described also by D. Norris et al. (Tetrahedron Letters 42 (2001), 4297-4299).

WO 92/22552 refers to imidazo[1,5-a]quinoxalines which are generally substituted at position 3 with a carboxylic acid group and derivatives thereof. These compounds are described to be useful as anxiolytic and sedative/hypnotic agents.

In contrast, only a limited number of imidazo[1,5-a]pyrido[3,2-e]pyrazines and their medical use are already published.

WO 99/45009 refers to a group of imidazopyrazines which are described to be inhibitors of protein tyrosine kinases used in the treatment of protein tyrosine kinase-associated disorders such as immunologic disorders.

### BRIEF DESCRIPTION OF THE DRAWINGS

FIG. 1 depicts the characterization of the collected proteins from FPLC by Western blot.

FIG. 2 depicts PDE 10 present in the membrane fraction.

FIG. 3 depicts the alignment of the pig PDE10 (SEQ ID NO: 5), guinea pig PDE10 (SEQ ID NO: 9), and rat PDE 10 (SEQ ID NO: 10) gene sequences to provide the consensus sequence (SEQ ID NO: 8).

FIG. 4 depicts the alignment of the pig PDE10 (SEQ ID NO: 11), guinea pig PDE10 (SEQ ID NO: 12), and rat PDE 10 (SEQ ID NO: 13) protein sequences within the catalytic domain to provide the consensus sequence (SEQ ID NO: 14).

FIG. 5 depicts the effect of the compounds of Example 91a, 35a, 95a and 55a on MK-801-induced psychosis.

FIG. 6 depicts the effect of the compounds of Example 38a and 47a on MK-801-induced psychosis

FIG. 7 depicts the effect of the compounds of Example 62a and 69a on MK-801-induced psychosis

FIG. 8 depicts the effect of the compounds of Example 29a and 30a on MK-801-induced psychosis.

FIG. 9 depicts the effect of the compounds of Example 1b and 11b on MK-801-induced psychosis.

### SUMMARY OF THE INVENTION

This invention relates to methods of reducing body weight or body fat as well as treating or preventing obesity, type 2 diabetes, metabolic syndrome, or glucose intolerance by administering compounds of formula (IIa) and their pharmaceutically acceptable salts, solvates and prodrugs.

Compounds of formula (IIa)

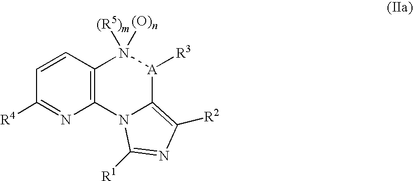

wherein the bond between A and N is a single bond or a double bond,
A is C when the bond is a double bond and CH when the bond is a single bond,
m is 0 or 1,
n is 0 or 1,
wherein R¹ and R² are independently selected from

### H,

a cyclic radical,
C₁₋₈ alkyl, optionally mono- or polysubstituted with halo, OH, O—C₁₋₃ alkyl and/or a cyclic radical,
C₂₋₈ alkenyl, optionally mono- or polysubstituted with halo, OH, O—C₁₋₃ alkyl and/or a cyclic radical,
C₂₋₈ alkynyl, optionally mono- or polysubstituted with halo, OH, O—C₁₋₃-alkyl and/or a cyclic radical,
a saturated, monounsaturated or polyunsaturated carboxylic ring system with 3 to 8 atoms, e.g. phenyl, or a heterocyclic ring system with 5 to 15 ring atoms containing at least one heteroatom selected from N including N-oxide, O and S, each optionally mono- or polysubstituted with halo, amino, C₁₋₃ alkylamino, di-C₁₋₃ alkylamino, nitro, C₁₋₃ alkyl, O—C₁₋₃ alkyl, and/or a cyclic radical, and
R³ is selected from

### H,

a cyclic radical,

### N₃,

### CN,

### R⁶, OR⁶, SR⁶, SOR⁶, SO₂R⁶,

### NH(CO)OR⁶, N((CO)OR⁶)₂, NR⁶((CO)OR⁶),

### NH—(C═O)—NH₂, NR⁶—(C═O)—NH₂,

### NH—(C═O)—NHR⁶, NR⁶—(C═O)—NHR⁶,

### NH—SO₂R⁶, N(SO₂R⁶)₂, and NR⁶(SO₂R⁶),

wherein R⁶ is in each case independently,
a cyclic radical,
C₁₋₈ alkyl, C₃₋₈ cyclo(hetero)alkyl,
C₂₋₈ alkenyl, C₃₋₈ cyclo(hetero)alkenyl,
or C₂₋₈ alkynyl each optionally mono or polysubstituted with halo, OH and/or O—C₁₋₃ alkyl, and/or a cyclic radical,
R⁷, OR⁷, SR⁷, NHSO₂R⁷, N(SO₂R⁷)₂, or N(R⁸)SO₂R⁷,
wherein R⁷ is aryl, heteroaryl, aryl-C₁₋₁₅ alkyl, heteroaryl-C₁₋₅ alkyl,
wherein aryl is phenyl or naphthyl, heteroaryl is an aromatic heterocyclic ring system of 5 to 15 ring atoms containing at least one atom selected from N including N-oxide, S, and O and wherein aryl and heteroaryl are optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical,
R³ is C₁₋₅ alkyl, optionally mono or polysubstituted with halo, OH, O—C₁₋₃ alkyl and/or a cyclic radical,
R⁴ is selected from

### H,

halo,
a cyclic radical,

### R⁹,

### OH or OR⁹,

NH(C═O)—C₁₋₃ alkyl, optionally mono- or polysubstituted with halo, OH, O—C₁₋₃ alkyl and/or a cyclic radical or

### NH₂, NHR⁹ or NR⁹R¹⁰,

wherein R⁹ and R¹⁰ are independently selected from
a cyclic radical,
C₁₋₆ alkyl or C₃₋₆ cyclo(hetero)alkyl, optionally mono- or polysubstituted with halo, OH, O—C₁₋₃ alkyl and/or a cyclic radical,
aryl-C₁₋₅-alkyl wherein aryl is phenyl, optionally mono- or polysubstituted with halo, amino, C₁₋₃ alkylamino, di-C₁₋₃ alkylamino, nitro, C₁₋₃ alkyl, OH, O—C₁₋₃ alkyl and/or a cyclic radical, or
NR⁹R¹⁰ together form a saturated or unsaturated five-, six- or seven-membered ring which can contain up to 3 heteroatoms, preferably N including N-oxide, S and/or O, optionally mono- or polysubstituted with halo, amino, C₁₋₃ alkylamino, di-C₁₋₃ alkylamino, C₁₋₃ alkyl, O—C₁₋₃ alkyl and/or aryl-C₁₋₅-alkyl, wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical,
   and R⁵ is selected from

### H,

C₁₋₅ alkyl, C₃₋₆ cycloalkyl or (CO)—C₁₋₅ alkyl, optionally mono or polysubstituted with halo, OH, O—C₁₋₃ alkyl and/or a cyclic radical,
or pharmaceutically acceptable salts and derivatives thereof.

A preferred embodiment of this invention relates to compounds of formula (IIa) wherein the bond between A and N is a double bond.

An other preferred embodiment of this invention relates to compounds of formula (IIa) wherein m and n are both 0.

A further preferred embodiment of this invention relates to compounds of formula (IIa) wherein R¹ is selected from

### H,

C₁₋₄ alkyl, particularly C₂₋₄ alkyl optionally mono- or polysubstituted with halo, OH, O—C₁₋₃ alkyl and/or a cyclic radical or
phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical.

Especially preferred are C₂₋₄-alkyl, e.g. propyl such as n-propyl or i-propyl, or phenyl, optionally substituted.

A further preferred embodiment of this invention relates to compounds of formula (IIa) wherein R² is

### H or

C₁₋₄ alkyl, particularly methyl, optionally substituted, e.g. halo substituted.

Especially preferred are hydrogen, a methyl group or a trifluoromethyl group.

A further preferred embodiment of this invention relates to compounds of formula (IIa) wherein R³ is H, CN or C₁₋₃ alkyl, e.g. methyl.

A further preferred embodiment of this invention relates to compounds of formula (IIa) wherein R³ is NH—(C═O)OR⁶, particularly NH—(C═O)—OC₁₋₅ alkyl, optionally mono- or polysubstituted as indicated above.

A further preferred embodiment of this invention relates to compounds of formula (IIa) wherein R³ is NH—SO₂R⁶, particularly NH—SO₂—C₁₋₅ alkyl, optionally mono- or polysubstituted as indicated above.

A further preferred embodiment of this invention relates to compounds of formula (IIa) wherein R⁴ is selected from

H, C₁₋₃ alkyl, O—C₁₋₃ alkyl, NH₂, NHC₁₋₃ alkyl, wherein alkyl is optionally mono- or polysubstituted with halo, OH, O—C₁₋₃ alkyl and/or a cyclic radical or
NH(C═O)—C₁₋₃ alkyl, optionally mono- or polysubstituted with halo, OH, O—C₁₋₃ alkyl and/or a cyclic radical or
cyclopropyl, cyclobutyl, tetrahydropyrrolyl, pyrrolyl, pyrazolyl, imidazolyl, 1,2,3-triazolyl, 1,2,4-triazolyl, piperidinyl, morpholinyl, piperazinyl, optionally mono- or polysubstituted with halo, OH, C₁₋₅ alkyl and/or O—C₁₋₃ alkyl, or aryl-C₁₋₅-alkyl, wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical, for example

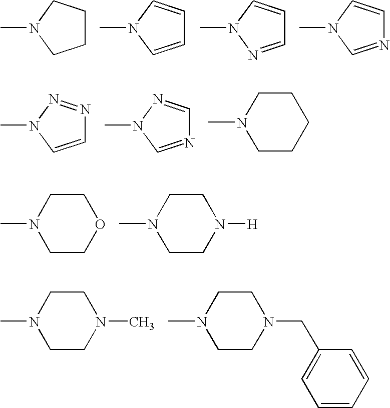

A further especially preferred embodiment of this invention relates to compounds of formula (IIa), wherein R⁴ is H, C₁₋₃ alkyl or O—C₁₋₃ alkyl, particularly H or OCH₃.

Examples of specific compounds of the formula (IIa) are the following:
4,8-dimethoxy-3-methyl-1-propyl-imidazo[1,5-a]pyrido[3,2-e]pyrazine
4,8-dimethoxy-1-propyl-imidazo[1,5-a]pyrido[3,2-e]pyrazine
4,8-dimethoxy-1-ethyl-3-methyl-imidazo[1,5-a]pyrido[3,2-e]pyrazine
4,8-dimethoxy-1,3-dimethyl-imidazo[1,5-a]pyrido[3,2-e]pyrazine
4,8-dimethoxy-3-methyl-imidazo[1,5-a]pyrido[3,2-e]pyrazine
1-ethyl-4-isopropyloxy-8-methoxy-3-methyl-imidazo[1,5-a]pyrido[3,2-e]pyrazine
1-ethyl-8-methoxy-3-methyl-4-propyloxy-imidazo[1,5-a]pyrido[3,2-e]pyrazine
4-cyclopentyloxy-1-ethyl-8-methoxy-3-methyl-imidazo[1,5-a]pyrido[3,2-e]pyrazine
4-isopropyloxy-8-methoxy-3-methyl-1-propyl-imidazo[1,5-a]pyrido[3,2-e]pyrazine
8-methoxy-1,3-dimethyl-4-(2,3,6-trifluorobenzyloxy)-imidazo[1,5-a]pyrido[3,2-e]pyrazine
4-(2,4-dichlorobenzyloxy)-1-ethyl-8-methoxy-3-methyl-imidazo[1,5-a]pyrido[3,2-e]pyrazine
4-(2-chloro-6-fluorobenzyloxy)-1-ethyl-8-methoxy-3-methyl-imidazo[1,5-a]pyrido[3,2-e]pyrazine
1-ethyl-8-methoxy-3-methyl-4-(2,3,6-trifluorobenzyloxy)-imidazo[1,5-a]pyrido[3,2-e]pyrazine
1-ethyl-8-methoxy-3-methyl-4-(2,4,6-trimethylbenzyloxy)-imidazo[1,5-a]pyrido[3,2-e]pyrazine
4-(2-chloro-6-fluorobenzyloxy)-8-methoxy-3-methyl-1-propyl-imidazo[1,5-a]pyrido[3,2-e]pyrazine
4-(2,6-difluorobenzyloxy)-8-methoxy-3-methyl-1-propyl-imidazo[1,5-a]pyrido[3,2-e]pyrazine
1-ethyl-8-methoxy-3-methyl-4-(2-phenylethyloxy)-imidazo[1,5-a]pyrido[3,2-e]pyrazine
8-methoxy-3-methyl-4-(2-phenylethyloxy)-1-propyl-imidazo[1,5-a]pyrido[3,2-e]pyrazine
8-methoxy-1,3-dimethyl-4-(2-phenylethyloxy)-imidazo[1,5-a]pyrido[3,2-e]pyrazine
8-methoxy-3-methyl-4-(2-phenylethyloxy)-imidazo[1,5-a]pyrido[3,2-e]pyrazine
8-methoxy-3-methyl-4-(3-phenylpropyloxy)-1-propyl-imidazo[1,5-a]pyrido[3,2-e]pyrazine
1-ethyl-8-methoxy-3-methyl-4-(3-phenylpropyloxy)-imidazo[1,5-a]pyrido[3,2-e]pyrazine
1,3-dimethyl-8-methoxy-4-(3-phenylpropyloxy)-imidazo[1,5-a]pyrido[3,2-e]pyrazine
4-[(3,5-dimethylisoxazol-4-yl)methyloxy]-1-ethyl-8-methoxy-3-methyl-imidazo[1,5-a]pyrido[3,2-e]pyrazine
1-ethyl-8-methoxy-3-methyl-4-methylthio-imidazo[1,5-a]pyrido[3,2-e]pyrazine
8-methoxy-3-methyl-4-methylthio-1-propyl-imidazo[1,5-a]pyrido[3,2-e]pyrazine
1,3-dimethyl-8-methoxy-4-methylthio-imidazo[1,5-a]pyrido[3,2-e]pyrazine
8-methoxy-3-methyl-4-methylthio-imidazo[1,5-a]pyrido[3,2-e]pyrazine 4-cyano-8-methoxy-3-methyl-1-propyl-imidazo[1,5-a]pyrido[3,2-e]pyrazine
4-cyano-8-methoxy-3-methyl-1-ethyl-imidazo[1,5-a]pyrido[3,2-e]pyrazine
4-azido-8-methoxy-3-methyl-1-propyl-imidazo[1,5-a]pyrido[3,2-e]pyrazine
8-methoxy-3-methyl-4-methylsulfinyl-1-propyl-imidazo[1,5-a]pyrido[3,2-e]pyrazine
8-methoxy-3-methyl-4-methylsulfonyl-1-propyl-imidazo[1,5-a]pyrido[3,2-e]pyrazine
1-ethyl-8-methoxy-3-methyl-4-methylsulfinyl-imidazo[1,5-a]pyrido[3,2-e]pyrazine
8-methoxy-3-methyl-1-propyl-imidazo[1,5-a]pyrido[3,2-e]pyrazine
1-ethyl-8-methoxy-3-methyl-imidazo[1,5-a]pyrido[3,2-e]pyrazine
4-ethyl-8-methoxy-3-methyl-1-propyl-imidazo[1,5-a]pyrido[3,2-e]pyrazine
3,4-dimethyl-8-methoxy-1-propyl-imidazo[1,5-a]pyrido[3,2-e]pyrazine
3,4-dimethyl-8-methoxy-1-propyl-imidazo[1,5-a]pyrido[3,2-e]pyrazine hydrochloride
1-ethyl-3,4-dimethyl-8-methoxy-imidazo[1,5-a]pyrido[3,2-e]pyrazine
1,3,4-trimethyl-8-methoxy-imidazo[1,5-a]pyrido[3,2-e]pyrazine
3,4-dimethyl-8-methoxy-1-(3,3,3-trifluoropropyl)-imidazo[1,5-a]pyrido[3,2-e]pyrazine
3,4-dimethyl-8-methoxy-1-pentyl-imidazo[1,5-a]pyrido[3,2-e]pyrazine
1-cyclohexyl-3,4-dimethyl-8-methoxy-imidazo[1,5-a]pyrido[3,2-e]pyrazine
3,4-dimethyl-1-hexyl-8-methoxy-imidazo[1,5-a]pyrido[3,2-e]pyrazine
3,4-dimethyl-8-methoxy-1-phenethyl-imidazo[1,5-a]pyrido[3,2-e]pyrazine
3,4-dimethyl-8-methoxy-1-phenyl-imidazo[1,5-a]pyrido[3,2-e]pyrazine
3,4-dimethyl-8-methoxy-1-phenyl-imidazo[1,5-a]pyrido[3,2-e]pyrazine dihydrochloride
3,4-dimethyl-8-methoxy-1-(2-chlorophenyl)-imidazo[1,5-a]pyrido[3,2-e]pyrazine
3,4-dimethyl-8-methoxy-1-(4-fluorophenyl)-imidazo[1,5-a]pyrido[3,2-e]pyrazine
1-propyl-3,4,8-trimethyl-imidazo[1,5-a]pyrido[3,2-e]pyrazine
1-propyl-3,4-dimethyl-imidazo[1,5-a]pyrido[3,2-e]pyrazine
1-propyl-4,8-dimethyl-imidazo[1,5-a]pyrido[3,2-e]pyrazine
8-difluoromethoxy-3,4-dimethyl-1-propyl-imidazo[1,5-a]pyrido[3,2-e]pyrazine
3,4-dimethyl-8-(piperidin-1-yl)-methoxy-1-propyl-imidazo[1,5-a]pyrido[3,2-e]pyrazine
3,4-dimethyl-8-(4-methyl-piperazin-1-yl)-methoxy-1-propyl-imidazo[1,5-a]pyrido[3,2-e]pyrazine
3,4-dimethyl-8-(2-ethyl-4-methyl-imidazol-1-yl)-methoxy-1-propyl-imidazo[1,5-a]pyrido[3,2-e]pyrazine
3,4-dimethyl-8-(2-propyl-4-methyl-imidazol-1-yl)-methoxy-1-propyl-imidazo[1,5-a]pyrido[3,2-e]pyrazine
4-difluoromethoxy-3-methyl-1-propyl-imidazo[1,5-a]pyrido[3,2-e]pyrazine-8-ol
8-methoxy-3-methyl-5-oxo-1-propyl-imidazo[1,5-a]pyrido[3,2-e]pyrazine 3,4-dimethyl-8-methoxy-5-oxo-1-propyl-imidazo[1,5-a]pyrido[3,2-e]pyrazine
8-methoxy-4-methoxycarbonylamino-3-methyl-1-propyl-imidazo[1,5-a]pyrido[3,2-e]pyrazine
4-ethoxycarbonylamino-8-methoxy-3-methyl-1-propyl-imidazo[1,5-a]pyrido[3,2-e]pyrazine
4-(N,N-bis-methoxycarbonyl)-amino-8-methoxy-3-methyl-1-propyl-imidazo[1,5-a]pyrido[3,2-e]pyrazine
8-methoxy-4-(methoxycarbonyl-methyl-amino)-3-methyl-1-propyl-imidazo[1,5-a]pyrido[3,2-e]pyrazine
8-methoxy-3-methyl-4-(3-methyl-ureido)-1-propyl-imidazo[1,5-a]pyrido[3,2-e]pyrazine
8-methoxy-3-methyl-1-propyl-4-ureido-imidazo[1,5-a]pyrido[3,2-e]pyrazine
8-methoxy-3-methyl-4-(3-isopropyl-ureido)-1-propyl-imidazo[1,5-a]pyrido[3,2-e]pyrazine
8-methoxy-3-methyl-4-methylsulfonylamino-1-propyl-imidazo[1,5-a]pyrido[3,2-e]pyrazine
4-(N,N-bis-methylsulfonyl)-amino-8-methoxy-3-methyl-1-propyl-imidazo[1,5-a]pyrido[3,2-e]pyrazine
4-ethylsulfonylamino-8-methoxy-3-methyl-1-propyl-imidazo[1,5-a]pyrido[3,2-e]pyrazine
1-ethyl-8-methoxy-3-methyl-4-methylsulfonylamino-imidazo[1,5-a]pyrido[3,2-e]pyrazine
8-methoxy-3-methyl-1-propyl-4-trifluoromethylsulfonylamino-imidazo[1,5-a]pyrido[3,2-e]pyrazine
8-methoxy-3-methyl-1-propyl-4-propylsulfonylamino-imidazo[1,5-a]pyrido[3,2-e]pyrazine
4-isopropylsulfonylamino-8-methoxy-3-methyl-1-propyl-imidazo[1,5-a]pyrido[3,2-e]pyrazine
8-methoxy-3-methyl-4-(4-methylphenylsulfonylamino)-1-propyl-imidazo[1,5-a]pyrido[3,2-e]pyrazine
4-[N,N-bis-(4-methylphenylsulfonyl)-amino]-8-methoxy-3-methyl-1-propyl-imidazo[1,5-a]pyrido[3,2-e]pyrazine
8-methoxy-3-methyl-1-(3,3,3-trifluoropropyl)-4-methylsulfonylamino-imidazo[1,5-a]pyrido[3,2-e]pyrazine
1-hexyl-8-methoxy-3-methyl-4-methylsulfonylamino-imidazo[1,5-a]pyrido[3,2-e]pyrazine
8-methoxy-3-methyl-1-phenethyl-4-methylsulfonylamino-imidazo[1,5-a]pyrido[3,2-e]pyrazine
8-methoxy-3-methyl-1-phenyl-4-methylsulfonylamino-imidazo[1,5-a]pyrido[3,2-e]pyrazine
1-(2-chlorophenyl)-8-methoxy-3-methyl-4-methylsulfonylamino-imidazo[1,5-a]pyrido[3,2-e]pyrazine
1-(4-fluorophenyl)-8-methoxy-3-methyl-4-methylsulfonylamino-imidazo[1,5-a]pyrido[3,2-e]pyrazine
3-methyl-8-(4-methyl-2-propyl-imidazol-1-yl)-1-propyl-4-methylsulfonylamino-imidazo[1,5-a]pyrido[3,2-e]pyrazine
3-methyl-4-methylsulfonylamino-1-propyl-imidazo[1,5-a]pyrido[3,2-e]pyrazin-8-ol hydrobromide
3-methyl-4-methylsulfonylamino-1-propyl-imidazo[1,5-a]pyrido[3,2-e]pyrazin-8-ol
8-difluoromethoxy-3-methyl-4-methylsulfonylamino-1-propyl-imidazo[1,5-a]pyrido[3,2-e]pyrazine
8-cyclopropylmethoxy-3-methyl-4-methylsulfonylamino-1-propyl-imidazo[1,5-a]pyrido[3,2-e]pyrazine
3-methyl-1-propyl-4,5-dihydro-imidazo[1,5-a]pyrido[3,2-e]pyrazine
8-methoxy-1-propyl-4,5-dihydro-imidazo[1,5-a]pyrido[3,2-e]pyrazine
8-methoxy-3-methyl-1-propyl-4,5-dihydro-imidazo[1,5-a]pyrido[3,2-e]pyrazine
8-methoxy-3-methyl-1-propyl-4,5-dihydro-imidazo[1,5-a]pyrido[3,2-e]pyrazine hydrochloride
1-ethyl-8-methoxy-3-methyl-4,5-dihydro-imidazo[1,5-a]pyrido[3,2-e]pyrazine
3,5-dimethyl-8-methoxy-1-propyl-4,5-dihydro-imidazo[1,5-a]pyrido[3,2-e]pyrazine
5-acetyl-8-methoxy-3-methyl-1-propyl-4,5-dihydro-imidazo[1,5-a]pyrido[3,2-e]pyrazine
   and their pharmaceutically acceptable salts and derivatives thereof.

Especially preferred, the compound of formula (IIa) is selected from 3,4-Dimethyl-8-methoxy-1-propyl-imidazo[1,5-a]-pyrido[3,2-e]-pyrazine and pharmaceutically acceptable salts and derivatives thereof.

This invention further relates to methods of reducing body weight or body fat as well as treating or preventing obesity, type 2 diabetes, metabolic syndrome, or glucose intolerance by administering compounds of formula (IIb) and to pharmaceutically acceptable salts, solvates and prodrugs thereof.

Compounds of formula (IIb)

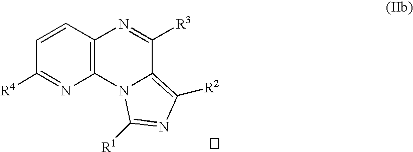

wherein R¹ and R² are independently selected from

### H,

a cyclic radical,
C₁₋₈ alkyl or C₃₋₈ cycloalkyl, optionally mono- or polysubstituted with halo, OH, O—C₁₋₃ alkyl, and/or a cyclic radical,
C₂₋₈ alkenyl or C₃₋₈ cycloalkenyl, optionally mono- or polysubstituted with halo, OH, O—C₁₋₃ alkyl and/or a cyclic radical,
C₂-C₈ alkynyl, optionally mono- or polysubstituted with halo, OH, O—C₁₋₃-alkyl, and/or a cyclic radical,
a saturated, monounsaturated or polyunsaturated heterocycle with 5 to 15 ring atoms, optionally mono- or polysubstituted with halo, amino, C₁₋₃ alkylamino, di-C₁₋₃ alkylamino, nitro, C₁₋₃ alkyl, and/or O—C₁₋₃ alkyl, and
phenyl, optionally mono- or polysubstituted with halo, amino, C₁₋₃ alkylamino, di-C₁₋₃ alkylamino, nitro, C₁₋₃ alkyl, and/or OC₁₋₃ alkyl and/or a cyclic radical,

R³ is NH₂, NHR⁵ or NR⁵R⁶;

wherein R⁵ and R⁶ are independently selected from
a cyclic radical,
C₁₋₅ alkyl, optionally mono- or polysubstituted with halo, OH, O—C₁₋₃ alkyl and/or a cyclic radical,
aryl-C₁₋₅-alkyl wherein aryl is phenyl, optionally mono- or polysubstituted with halo, nitro, C₁₋₃ alkyl, OC₁₋₃ alkyl, and/or a cyclic radical,
(C═O)—C₁₋₅ alkyl optionally mono- or polysubstituted with halo, OH, O—C₁₋₃ alkyl and/or a cyclic radical,
or NR⁵R⁶ together form a saturated or unsaturated five-, six- or seven-membered ring which can contain up to 3 heteroatoms, preferably N including N-oxide, S and O, optionally mono- or polysubstituted with halo, C₁₋₃ alkyl, O—C₁₋₃ alkyl and/or aryl-C₁₋₅-alkyl, wherein aryl is phenyl, optionally mono- or polysubstituted with halo, nitro, C₁₋₃ alkyl, and/or O—C₁₋₃ alkyl, and/or a cyclic radical, and
R⁴ is selected from

### H,

halo,
a cyclic radical,

### R⁷,

### OH or OR⁷,

NH(C═O)—C₁₋₃ alkyl, optionally mono- or polysubstituted with halo, OH, O—C₁₋₃ alkyl and/or a cyclic radical, in particular aryl or phenyl, or

### NH₂, NHR⁷ or NR⁷R⁸,

wherein R⁷ and R⁸ are independently selected from
a cyclic radical,
C₁₋₆ alkyl or C₃₋₆ cycloalkyl, optionally mono- or polysubstituted with halo, OH, O—C₁₋₃ alkyl, and/or a cyclic radical,
aryl-C₁₋₅-alkyl wherein aryl is phenyl, optionally mono- or polysubstituted with halo, nitro, C₁₋₃ alkyl, O—C₁₋₃ alkyl, and/or a cyclic radical,
or NR⁷R⁸ together form a saturated or unsaturated five- or six-membered ring which can contain up to 3 heteroatoms, preferably N including N-oxide, S and O, optionally mono- or polysubstituted with halo, C₁₋₃ alkyl, C₃₋₆ cycloalkyl, O—C₁₋₃ alkyl and/or aryl-C₁₋₅-alkyl, wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical,
or pharmaceutically acceptable salts and derivatives thereof.

A preferred embodiment of this invention relates to compounds of formula (IIb) wherein R¹ is selected from

### H,

C₁₋₄ alkyl, particularly C₂₋₄ alkyl optionally mono- or polysubstituted with halo, OH, C₁₋₃ alkyl, or/and a cyclic radical or
phenyl, optionally mono- or polysubstituted with halo, amino, C₁₋₃ alkylamino, di-C₁₋₃ alkylamino, nitro, C₁₋₃ alkyl, O—C₁₋₃ alkyl or/and a cyclic radical.

Especially preferred are C₂₋₄-alkyl or phenyl.

An other preferred embodiment of this invention relates to compounds of formula (IIb) wherein R² is

### H or

C₁₋₄ alkyl optionally halogenated, particularly methyl or trifluoromethyl.

Especially preferred are hydrogen or a methyl-group.

A further preferred embodiment of this invention relates to compounds of formula (IIb) wherein R³ is selected from

### NH₂,

NHC₁₋₃ alkyl, optionally mono- or polysubstituted with halo, OH, O—C₁₋₃ alkyl and/or a cyclic radical, or
NH(C═O)—C₁₋₃ alkyl, optionally mono- or polysubstituted with halo, OH, O—C₁₋₃ alkyl and/or a cyclic radical or
cyclopropyl, cyclobutyl, tetrahydropyrrolyl, pyrrolyl, pyrazolyl, imidazolyl, 1,2,3-triazolyl, 1,2,4-triazolyl, piperidinyl, morpholinyl, piperazinyl, optionally substituted with C₁₋₃ alkyl, optionally mono- or polysubstituted with halo, OH and/or O—C₁₋₃ alkyl, or arylalkyl, wherein aryl is phenyl, optionally mono- or polysubstituted with halo, amino, C₁₋₃ alkylamino, di-C₁₋₃ alkylamino, nitro, C₁₋₃ alkyl, and/or O—C₁₋₃ alkyl and/or a cyclic radical, for example

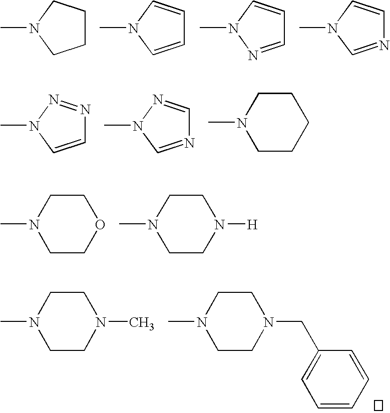

Especially preferred is one of —NH₂, —NH—C₁₋₃-alkyl, —NH—(C═O)—C₁₋₃-alkyl or -imidazolyl.

Also a preferred embodiment of this invention relates to compounds of formula (IIb) wherein R⁴ is selected from

OH or O—C₁₋₃ alkyl, optionally mono- or polysubstituted with halo, OH, O—C₁₋₃ alkyl, and/or a cyclic radical,
NHC₁₋₃ alkyl, optionally mono- or polysubstituted with halo, OH, O—C₁₋₃ alkyl and/or a cyclic radical, or
NH benzyl, wherein the phenyl group is phenyl, optionally mono- or polysubstituted with halo, amino, C₁₋₃ alkylamino, di-C₁₋₃ alkylamino, nitro,
C₁₋₃ alkyl, O—C₁₋₃ alkyl and/or a cyclic radical or
cyclopropyl, cyclobutyl, tetrahydropyrrolyl, pyrrolyl, pyrazolyl, imidazolyl, 1,2,3-triazolyl, 1,2,4-triazolyl, piperidinyl, morpholinyl, piperazinyl, optionally substituted with C₁₋₃ alkyl, optionally mono- or polysubstituted with halo, OH, C₁₋₅ alkyl and/or O—C₁₋₃ alkyl, or arylalkyl, wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical.

Especially preferred are one of hydrogen, —O—C₁₋₃-alkyl, —NH—C₁₋₃-alkyl, —NH-benzyl or the following groups:

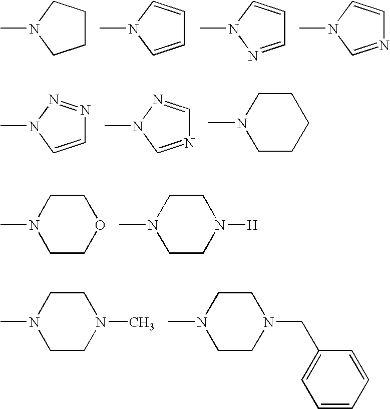

The compounds of formula (IIb) are inhibitors of phosphodiesterase 10 and thus have new biological properties. Based on these properties therapeutic uses of compounds of formula (IIb) which are different from those disclosed in WO 99/45009 are part of this invention.

Examples of specific compounds of the formula (IIb) are the following:
4-amino-8-methoxy-3-methyl-1-propyl-imidazo[1,5-a]pyrido[3,2-e]pyrazine
4-amino-1-ethyl-8-methoxy-3-methyl-imidazo[1,5-a]pyrido[3,2-e]pyrazine
4-amino-1-ethyl-3-methyl-imidazo[1,5-a]pyrido[3,2-e]pyrazine
4-amino-3-methyl-1-propyl-imidazo[1,5-a]pyrido[3,2-e]pyrazine
4-amino-1-ethyl-8-(2-ethyl-4-methyl-imidazol-1-yl)-3-methyl-imidazo[1,5-a]pyrido[3,2-e]pyrazine
4-amino-3-methyl-1-propyll-8-(2-propyl-4-methyl-imidazol-1-yl)-imidazo[1,5-a]pyrido[3,2-e]pyrazine
4-amino-1-hexyl-8-methoxy-3-methyl-imidazo[1,5-a]pyrido[3,2-e]pyrazine
4-amino-8-methoxy-3-methyl-1-(3,3,3-trifluoropropyl)-imidazo[1,5-a]pyrido[3,2-e]pyrazine
4-amino-8-methoxy-3-methyl-1-phenethyl-imidazo[1,5-a]pyrido[3,2-e]pyrazine
4-amino-8-methoxy-3-methyl-1-phenyl-imidazo[1,5-a]pyrido[3,2-e]pyrazine
4-amino-1-(2-chloro-phenyl)-8-methoxy-3-methyl-imidazo[1,5-a]pyrido[3,2-e]pyrazine
4-amino-1-(4-fluoro-phenyl)-8-methoxy-3-methyl-imidazo[1,5-a]pyrido[3,2-e]pyrazine
4-amino-1-isopropyl-8-methoxy-imidazo[1,5-a]pyrido[3,2-e]pyrazine
4-amino-8-methoxy-imidazo[1,5-a]pyrido[3,2-e]pyrazine
4-amino-8-methoxy-3-phenyl-imidazo[1,5-a]pyrido[3,2-e]pyrazine
4-(N-methyl-amino)-8-methoxy-3-methyl-1-propyl-imidazo[1,5-a]pyrido[3,2-e]pyrazine
4-(N-ethyl-amino)-8-methoxy-3-methyl-1-propyl-imidazo[1,5-a]pyrido[3,2-e]pyrazine
4-(N-methyl-amino)-1-ethyl-8-methoxy-3-methyl-imidazo[1,5-a]pyrido[3,2-e]pyrazine
4-(N,N-dimethyl-amino)-8-methoxy-3-methyl-1-propyl-imidazo[1,5-a]pyrido[3,2-e]pyrazine
4-(N-butyl-amino)-1-ethyl-8-methoxy-3-methyl-imidazo[1,5-a]pyrido[3,2-e]pyrazine
4-(N-benzyl-amino)-1-ethyl-8-methoxy-3-methyl-imidazo[1,5-a]pyrido[3,2-e]pyrazine
4-(N-cyclopentyl-amino)-1-ethyl-8-methoxy-3-methyl-imidazo[1,5-a]pyrido[3,2-e]pyrazine
4-(N-cyclopentyl-amino)-8-methoxy-3-methyl-1-propyl-imidazo[1,5-a]pyrido[3,2-e]pyrazine
1-ethyl-8-methoxy-3-methyl-4-morpholino-imidazo[1,5-a]pyrido[3,2-e]pyrazine
4-azetidine-8-methoxy-3-methyl-1-(3,3,3-trifluoropropyl)-imidazo[1,5-a]pyrido[3,2-e]pyrazine
8-methoxy-3-methyl-1-propyl-4-pyrrolidino-imidazo[1,5-a]pyrido[3,2-e]pyrazine
8-methoxy-3-methyl-4-piperidino-1-propyl-imidazo[1,5-a]pyrido[3,2-e]pyrazine
1-ethyl-8-methoxy-3-methyl-4-(4-phenylpiperazino)-imidazo[1,5-a]pyrido[3,2-e]pyrazine
8-methoxy-3-methyl-1-propyl-4-(pyrazol-1-yl)-imidazo[1,5-a]pyrido[3,2-e]pyrazine
8-methoxy-3-methyl-1-propyl-4-(pyrazol-1-yl)-imidazo[1,5-a]pyrido[3,2-e]pyrazine hydro chloride
4-(imidazol-1-yl)-8-methoxy-3-methyl-1-propyl-imidazo[1,5-a]pyrido[3,2-e]pyrazine
8-methoxy-3-methyl-1-propyl-4-(1,2,3-triazol-1-yl)-imidazo[1,5-a]pyrido[3,2-e]pyrazine
8-methoxy-3-methyl-1-propyl-4-(1,2,4-triazol-1-yl)-imidazo[1,5-a]pyrido[3,2-e]pyrazine
8-methoxy-3-methyl-4-(2-methyl-imidazol-1-yl)-1-propyl-imidazo[1,5-a]pyrido[3,2-e]pyrazine
4-(imidazol-1-yl)-3-methyl-1-propyl-imidazo[1,5-a]pyrido[3,2-e]pyrazine-8-ol
1-ethyl-4-(N-formyl-amino)-8-methoxy-3-methyl-imidazo[1,5-a]pyrido[3,2-e]pyrazine
4-(N-formyl-amino)-8-methoxy-3-methyl-1-propyl-imidazo[1,5-a]pyrido[3,2-e]pyrazine
4-(N-acetyl-amino)-8-methoxy-3-methyl-1-propyl-imidazo[1,5-a]pyrido[3,2-e]pyrazine
4-(N,N-diacetyl-amino)-8-methoxy-3-methyl-1-propyl-imidazo[1,5-a]pyrido[3,2-e]pyrazine
4-(N-acetyl-amino)-1-ethyl-8-methoxy-3-methyl-imidazo[1,5-a]pyrido[3,2-e]pyrazine
4-(N,N-diacetyl-amino)-1-ethyl-8-methoxy-3-methyl-imidazo[1,5-a]pyrido[3,2-e]pyrazine
4-(N-acetyl-amino)-8-methoxy-3-methyl-1-phenyl-imidazo[1,5-a]pyrido[3,2-e]pyrazine
8-methoxy-3-methyl-4-(N-propionyl-amino)-1-propyl-imidazo[1,5-a]pyrido[3,2-e]pyrazine
4-(N-cyclopropylcarboxy-amino)-8-methoxy-3-methyl-1-propyl-imidazo[1,5-a]pyrido[3,2-e]pyrazine
   and pharmaceutically acceptable salts and derivatives thereof.

Further, it was found that compounds of formula (IIc)

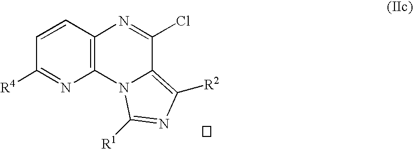

wherein X is Cl or Br and R¹, R² and R⁴ are as defined above are potent inhibitors of phosphodiesterase 10.

The term “halo” refers to fluoro, chloro, bromo or iodo.

The terms “alkyl”, “alkenyl” and “alkynyl” refer to straight or branched hydrocarbon radicals with up to 8 carbon atoms preferably up to 6 carbon atoms and more preferably up to 5 carbon atoms such as methyl, ethyl, vinyl, ethynyl, propyl, isopropyl, allyl, propynyl, butyl, isobutyl, t-butyl, butenyl, butynyl etc. which may optionally be substituted as indicated above. “Alkyl” groups are saturated; an “alkenyl” group contains at least one double carbon-carbon bond; and an “alkynyl” group contains at least one triple carbon-carbon bond.

As used herein, “cyclic radical” refers to a saturated, unsaturated, or aromactic carbocycle or heterocycle, optionally mono- or polysubstituted with halo, amino, C₁₋₃ alkylamino, di-C₁₋₃ alkylamino, nitro, C₁₋₃ alkyl, OH, or O—C₁₋₃ alkyl. The cyclic radical can be a 3 to 24 membered mono- or polycyclic ring. In some embodiments, the cyclic radical is a 3-, 4-, 5-, 6-, or 7-membered ring. The cyclic radical can contain 3 to 20, or in some embodiments, 4 to 10 ring forming carbon atoms. The cyclic radical includes cyclo(hetero)alkyl, aryl and heteroaryl groups as defined below.

“Cyclo(hetero)alkyl” refers to both cycloalkyl and cycloheteroalkyl groups. Cycloheteroalkyl and heteroaryl groups may, for example, contain 1 to 6, or in some embodiments, 1 to 3 ring forming heteroatoms, selected from O, N, S, and/or P. The cyclic radical can be bound via a carbon atom or optionally via a N, O, S, SO, or SO₂ group. An example of an aryl cyclic radical is phenyl. Examples of cycloalkyl cyclic radicals include cyclopropyl, cyclobutyl, cyclopentyl, cyclohexyl, and cycloheptyl. Examples of heteroaryl cyclic radicals include thienyl, furanyl, pyrrolyl, imidazolyl, triazolyl, oxazolyl, isoxazolyl, pyrazolyl, thiazolyl, pyridinyl, pyrimidinyl, and the like. Examples of cycloheteroalkyl cyclic radicals include pyrrolidinyl, tetrahydrofuranyl, morpholino, thiomorpholino, piperazinyl, tetrahydrothienyl, 2,3-dihydrobenzofuryl, 1,3-benzodioxole, benzo-1,4-dioxane, piperidinyl, isoxazolidinyl, isothiazolidinyl, pyrazolidinyl, oxazolidinyl, thiazolidinyl, and imidazolidinyl. Examples of heteroaryl groups are provided below.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, and the like. In some embodiments, an aryl group has from 6 to about 20 carbon atoms.

As used herein, “arylalkyl” refers to an alkyl group substituted by an aryl group. Example arylalkyl groups include benzyl and phenylethyl.

As used herein, “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems, including spirocycles. In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Also included in the definition of cycloalkyl are moieties that have one or more aromatic rings fused (i.e., having a bond in common with) to the cycloalkyl ring, for example, benzo derivatives of cyclopentane, cyclopentene, cyclohexane, and the like. A cycloalkyl group having one or more fused aromatic rings can be attached through either the aromatic or non-aromatic portion. One or more ring-forming carbon atoms of a cycloalkyl group can be oxidized, for example, having an oxo or sulfido substituent. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like.

As used herein, a “heteroaryl” group refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, a “heteroarylalkyl” group refers to an alkyl group substituted by a heteroaryl group. An example of a heteroarylalkyl group is pyridylmethyl.

As used herein, “cycloheteroalkyl” refers to a non-aromatic heterocycle where one or more of the ring-forming atoms is a heteroatom such as an O, N, or S atom. Cycloheteroalkyl groups can include mono- or polycyclic (e.g., having 2, 3 or 4 fused rings) ring systems as well as spirocycles. Example cycloheteroalkyl groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of cycloheteroalkyl are moieties that have one or more aromatic rings fused (i.e., having a bond in common with) to the nonaromatic heterocyclic ring, for example phthalimidyl, naphthalimidyl, and benzo derivatives of heterocycles. A cycloheteroalkyl group having one or more fused aromatic rings can be attached though either the aromatic or non-aromatic portion. Also included in the definition of cycloheteroalkyl are moieties where one or more ring-forming atoms is substituted by 1 or 2 oxo or sulfido groups. In some embodiments, the cycloheteroalkyl group has from 1 to about 20 carbon atoms, and in further embodiments from about 3 to about 20 carbon atoms. In some embodiments, the cycloheteroalkyl group contains 3 to about 20, 3 to about 14, 3 to about 7, or 5 to 6 ring-forming atoms. In some embodiments, the cycloheteroalkyl group has 1 to about 4, 1 to about 3, or 1 to 2 heteroatoms. In some embodiments, the cycloheteroalkyl group contains 0 to 3 double bonds. In some embodiments, the cycloheteroalkyl group contains 0 to 2 triple bonds.

As used herein, the term “substituted” refers to the replacement of a hydrogen moiety with a non-hydrogen moiety in a molecule or group. A molecule or group may be monosubstituted. A molecule or group may be also polysubstituted with the same or different substituents. A substituent may comprise a single non-hydrogen moiety or a combination of more than one non-hydrogen moieties, e.g., halo and C₁₋₃ alkyl, thus being a C₁₋₃ halo alkyl substituent.

The invention furthermore relates to the physiologically acceptable salts, solvates and derivatives of the compounds according to formulas (IIa), (IIb), and (IIc). Derivatives of the compounds according to formula (IIa), (IIb), and (IIc) are, for example, amides, esters and ethers. Further, the term “derivative” also encompasses prodrugs and metabolites of compounds of formula (IIa), (IIb), and (IIc).

The physiologically acceptable salts may be obtained by neutralizing the bases with inorganic or organic acids or by neutralizing the acids with inorganic or organic bases. Examples of suitable inorganic acids are hydrochloric acid, sulphuric acid, phosphoric acid or hydrobromic acid, while examples of suitable organic acids are carboxylic acid, sulpho acid or sulphonic acid, such as acetic acid, tartaric acid, lactic acid, propionic acid, glycolic acid, malonic acid, maleic acid, fumaric acid, tannic acid, succinic acid, alginic acid, benzoic acid, 2-phenoxybenzoic acid, 2-acetoxybenzoic acid, cinnamic acid, mandelic acid, citric acid, maleic acid, salicylic acid, 3-aminosalicylic acid, ascorbic acid, embonic acid, nicotinic acid, isonicotinic acid, oxalic acid, gluconic acid, amino acids, methanesulphonic acid, ethanesulphonic acid, 2-hydroxyethanesulphonic acid, ethane-1,2-disulphonic acid, benzenesulphonic acid, 4-methylbenzenesulphonic acid or naphthalene-2-sulphonic acid. Examples of suitable inorganic bases are sodium hydroxide, potassium hydroxide and ammonia, while examples of suitable organic bases are amines, preferably, however, tertiary amines, such as trimethylamine, triethylamine, pyridine, N,N-dimethylaniline, quinoline, isoquinoline, α-picoline, β-picoline, γ-picoline, quinaldine and pyrimidine.

In addition, physiologically acceptable salts of the compounds according to formula (IIa), (IIb), and (IIc) can be obtained by converting derivatives which possess tertiary amino groups into the corresponding quaternary ammonium salts in a manner known per se using quaternizing agents. Examples of suitable quaternizing agents are alkyl halides, such as methyl iodide, ethyl bromide and n-propyl chloride, and also arylalkyl halides, such as benzyl chloride or 2-phenylethyl bromide.

Furthermore, in the case of the compounds of the formula (IIa), (IIb), and (IIc) which contain an asymmetric carbon atom, the invention relates to the D form, the L form and D,L mixtures and also, where more than one asymmetric carbon atom is present, to the diastereomeric forms. Those compounds of the formula (II) which contain asymmetric carbon atoms, and which as a rule accrue as racemates, can be separated into the optically active isomers in a known manner, for example using an optically active acid. However, it is also possible to use an optically active starting substance from the outset, with a corresponding optically active or diastereomeric compound then being obtained as the end product.

The compounds according to the invention have been found to have pharmacologically important properties which can be used therapeutically. The compounds according to formula (IIa), (IIb), and (IIc) can be used alone, in combination with each other or in combination with other active compounds. The compounds according to the invention are inhibitors of phosphodiesterase 10. It is therefore a part of the subject-matter of this invention that the compounds according to formula (IIa), (IIb), and (IIc), and their salts and also pharmaceutical preparations which comprise these compounds or their salts, can be used for treating or preventing discorders associated with, accompanied by and/or covered by phosphodiesterase hyperactivity and/or disorders in which inhibiting phosphodiesterase 10 is of value.

Surprisingly, the compounds of formula (IIa), (IIb), and (IIc) are potent inhibitors of the enzyme PDE10.

It is an embodiment of this invention, that compounds of formula (IIa), (IIb), and (IIc) including their salts, solvates and prodrugs and also pharmaceutical compositions comprising an amount of a compound of formula (IIa), (IIb), and (IIc) or one of its salts, solvates or prodrugs effective in inhibiting PDE10 can be used for the treatment of central nervous system disorders of mammals including a human.

More particularly, the invention relates to the treatment of neurological and psychiatric disorders including, but not limited to, (1) schizophrenia and other psychotic disorders; (2) mood [affective] disorders; (3) neurotic, stress-related and somatoform disorders including anxiety disorders; (4) eating disorders; sexual dysfunction comprising excessive sexual drive; (5) disorders of adult personality and behaviour; (6) disorders usually first diagnosed in infancy, childhood and adolescence; (7) mental retardation and (8) disorders of psychological development; (9) disorders comprising the symptom of cognitive deficiency in a mammal, including a human; (10) factitious disorders.

(1) Examples of schizophrenia and other psychotic disorders that can be treated according to the present invention include, but are not limited to, continuous or episodic schizophrenia of different types (for instance paranoid, hebephrenic, catatonic, undifferentiated, residual, and schizophreniform disorders); schizotypal disorders (such as borderline, latent, prepsychotic, prodromal, pseudoneurotic pseudopsychopathic schizophrenia and schizotypal personality disorder); persistent delusional disorders; acute, transient and persistent psychotic disorders; induced delusional disorders; schizoaffective disorders of different type (for instance manic depressive or mixed type); puerperal psychosis and other and unspecified nonorganic psychosis.

(2) Examples of mood [affective] disorders that can be treated according to the present invention include, but are not limited to, manic episodes associated to bipolar disorder and single manic episodes, hypomania, mania with psychotic symptoms; bipolar affective disorders (including for instance bipolar affective disorders with current hypomanic and manic episodes with or without psychotic symptoms, bipolar I disorder or bipolar II disorder); depressive disorders, such as single episode or recurrent major depressive disorder of the mild moderate or severe type, depressive disorder with postpartum onset, depressive disorders with psychotic symptoms; persistent mood [affective] disorders, such as cyclothymia, dysthymia; premenstrual dysphoric disorder.

(3) Examples of disorders belonging to the neurotic, stress-related and somatoform disorders that can be treated according to the present invention include, but are not limited to, phobic anxiety disorders, for instance agoraphobia and social phobia primarily but not exclusively related to psychosis; other anxiety disorders such as panic disorders and general anxiety disorders; obsessive compulsive disorder; reaction to sever stress and adjustment disorders, such as post traumatic stress disorder; dissociative disorders and other neurotic disorders such as depersonalisation-derealisation syndrome.

(5) Examples of disorders of adult personality and behaviour that can be treated according to the present invention include, but are not limited to, specific personality disorders of the paranoid, schizoid, schizotypal, antisocial, borderline, histrionic, narcissistic, avoidant, dissocial, emotionally unstable, anankastic, anxious and dependent type; mixed personality disorders; habit and impulse disorders (such as trichotillomania, pyromania, maladaptive aggression); disorders of sexual preference.

(6) Examples of disorders usually first diagnosed in infancy, childhood and adolescence that can be treated according to the present invention include, but are not limited to, hyperkinetic disorders, attentional deficit/hyperactivity disorder (AD/HD), conduct disorders; mixed disorders of conduct and emotional disorders; nonorganic enuresis, nonorganic encopresis; stereotyped movement disorder; and other specified behavioural emotional disorders, such as attention deficit disorder without hyperactivity, excessive masturbation nail-biting, nose-picking and thumb-sucking; disorders of psychological development particularly schizoid disorder of childhood and pervasive development disorders such as psychotic episodes associated to Asperger's syndrome.

(7) Exemplary neurological disorders include neurodegenerative disorders including, without being limited to, Parkinson's disease, Huntington's disease, dementia (for example Alzheimer's disease, multi-infarct dementia, AIDS-related dementia, or Fronto temperal dementia), neurodegeneration associated with cerebral trauma, neurodegeneration associated with stroke, neurodegeneration associated with cerebral infarct, hypoglycemia-induced neurodegeneration, neurodegeneration associated with epileptic seizure, neurodegeneration associated with neurotoxic poisoning or multi-system atrophy.

(8) Examples of disorders of psychological development include but are not limited to developmental disorders of speech and language, developmental disorders of scholastic skills, such as specific disorder of arithmetical skills, reading disorders and spelling disorders and other learning disorders. These disorders are predominantly diagnosed in infancy, childhood and adolescence.

(9) The phrase “cognitive deficiency” as used here in “disorder comprising as a symptom cognitive deficiency” refers to a subnormal functioning or a suboptimal functioning in one or more cognitive aspects such as memory, intellect, learning and logic ability, or attention in a particular individual comparative to other individuals within the same general age population.

(10) Examples of disorders comprising as a symptom cognitive deficiency that can be treated according to the present invention include, but are not limited to, cognitive deficits primarily but not exclusively related to psychosis including schizophrenia; depression; age-associated memory impairment, autism, autistic spectrum disorders, fragile X syndrome, Parkinson's disease, Alzheimer's disease, multi infarct dementia, spinal cord injury, CNS hypoxia, Lewis body dementia, stroke, frontotemporal dementia, progressive supranuclear palsy Huntington's disease and in HIV disease, cerebral trauma, cardiovascular disease, drug abuse, diabetes associated cognitive impairment, and mild cognitive disorder.

(11) Additionally, the invention relates to movement disorders with malfunction of basal ganglia. Examples of movement disorders with malfunction of basal ganglia that can be treated according to the present invention include, but are not limited to, different subtypes of dystonia, such as focal dystonias, multiple-focal or segmental dystonias, torsion dystonia, hemispheric, generalised and tardive dyskinesias (induced by psychopharmacological drugs), akathisias, dyskinesias such as Huntington's disease, Parkinson's disease, Lewis body disease, restless leg syndrome, PLMS.

(12) Furthermore the invention relates to the treatment of organic, including symptomatic mental disorders, especially to organic delusional (schizophrenia-like) disorders, presenil or senile psychosis associated to dementia, to psychosis in epilepsy and Parkinson's disease and other organic and symptomatic psychosis; delirium; infective psychosis; personality and behavioural disorders due to brain disease, damage and dysfunction.

(13) The invention relates to the treatment of mental and behavioural disorders due to psychoactive compounds, more particular to the treatment of psychotic disorders and residual and late-onset psychotic disorders induced by alcohol, opioids, cannabinoids, cocaine, hallucinogens, other stimulants, including caffeine, volatile solvents and other psychoactive compounds.

(14) The invention further relates to a general improvement of learning and memory capacities in a mammal, including a human.

Compounds currently used to treat schizophrenia have been associated with several undesirable side effects. These side effects include weight gain, hyperprolactinemia, elevated triglyceride levels, metabolic syndrome (markers: diabetes, hyperlipidemia, hypertension, and obesity), glucose abnormalities (such as hyperglycemia, elevated blood glucose and impaired glucose tolerance), and the exhibition of extrapyramidal symptoms. The weight gain observed with conventional atypical antipsychotics, such as risperidone and olanzapine, has been associated with an increased risk of cardiovascular disease and diabetes mellitus.

Compounds of the present invention are useful in treating schizophrenia to effect a clinically relevant improvement such as reduction of a PANSS total score in a patient, while maintaining body weight, maintaining or improving glucose levels and/or tolerance, maintaining and/or improving triglycerides levels and/or total cholesterol levels and/or maintaining an EPS profile similar to baseline measurements before administration.

The PDE10 inhibitors of the invention are further useful in the prevention and treatment of obesity, type 2 diabetes (non-insulin dependent diabetes), metabolic syndrome, glucose intolerance, and related health risks, symptoms or disorders. As such, the compounds can also be used to reduce body fat or body weight of an overweight or obese individual. In some embodiments, the PDE10 inhibitor is selective for PDE10, meaning that it is a better inhibitor of PDE10 than for any other PDE. In some embodiments, the selective PDE10 inhibitor can reduce PDE10 activity at least 10-fold or at least 100-fold compared to other PDE's.

As used herein, the terms “overweight” and “obese” are meant to refer to adult persons 18 years or older having a greater than ideal body weight (or body fat) measured by the body mass index (BMI). BMI is calculated by weight in kilograms divided by height in meters squared (kg/m²) or, alternatively, by weight in pounds, multiplied by 703, divided by height in inches squared (lbs×703/in²). Overweight individuals typically have a BMI of between 25 and 29, whereas obese individuals typically have a BMI of 30 or more (see, e.g., National Heart, Lung, and Blood institute, Clinical Guidelines on the Identification, Evaluation, and Treatment of Overweight and Obesity in Adults, The Evidence Report, Washington, D.C.:U.S. Department of Health and Human Services, NIH publication no. 98-4083, 1998). Other means for indicating excess body weight, excess body fat, and obesity include direct measure of body fat and/or waist-to-hip ratio measurements.

The term “metabolic syndrome” is used according to its usual meaning in the art. The American Heart Association characterizes metabolic syndrome as having at least 3 of the 5 below symptoms: 1) Elevated waist circumference (>102 cm (40 inches) in men; >88 cm (35 inches) in women), 2) Elevated triglycerides (>150 mg/dL (>1.7 mmol/L) or drug treatment for elevated triglycerides), 3) Reduced HDL-C (<40 mg/dL (1.03 mmol/L) in men <50 mg/dL (1.3 mmol/L) in women or drug treatment for reduced HDL-C, 4) Elevated blood pressure (>130/85 mmHg or drug treatment for hypertension), and 5) Elevated fasting glucose (>100 mg/dL or drug treatment for elevated glucose). See, Grundy, S. M. et al., Circulation, 2005, 112(17, e285 (online at circ.ahajournals.org/cgi/reprint/112/17/e285). Metabolic syndrome according to the World Health Organization (See, Alberti et al., Diabet. Med. 15, 539-553, 1998) includes individuals suffering from diabetes, glucose intolerance, low fasting glucose, or insulin resistance plus two or more of 1) High blood pressure (>160/90 mmHg), 2) Hyperlipdemia (triglycerides≧150 mg/dL or HDL cholesterol<35 mg/dL in men and <39 mg/dL in women), 3) Central obesity (waist-to-hip ratio of >0.90 for men and >0.85 for women or BMI>30 kg/m2), and 4) Microalbuminuria (urinary albumin excretion rate ≧20 μg/min or an albumin-to-creatine ratio≧20 μg/kg).

The present methods relating to reduction of body fat or body weight, as well as the treatment or prevention of obesity, type 2 diabetes (non-insulin dependent diabetes), metabolic syndrome, glucose intolerance, and related health risks, symptoms or disorders can be carried out by the administration of one or more compounds of the present invention. In some embodiments, one or more additional therapeutic agents can be administered such as anti-obesity agents. Example anti-obesity agents include apolipoprotein-B secretion/microsomal triglyceride transfer protein(apo-B/MTP) inhibitors, 11-beta-hydroxysteroid dehydrogenase-1 (11beta-HSD type 1) inhibitors, peptide YY3-36 or analogs thereof, MCR-4 agonists, cholecystokinin-A (CCK-A) agonists, monoamine reuptake inhibitors (such as sibutramine), cannabinoid receptor-1 antagonists (such as rimona an, sympathomimetic agents, P3 adrenergic receptor agonists, 5 dopamine agonists; (such as bromocriptine), melanocyte-stimulating hormone receptor analogs, 5HT_{2C} agonists, melanin concentrating hormone antagonists, leptin (the OB protein), leptin analogs, leptin receptor agonists, galanin antagonists, lipase inhibitors (such as tetrahydrolipstatin, i.e. orlistat), anorectic agents (such as a bombesin agonist), neuropeptide-Y receptor antagonists (e.g., NPY Y5 receptor antagonists, such as the compounds described in U.S. Pat. Nos. 6,566,367; 6,1649,624; 6,1638,942; 6,1605,720; 6,1495,569; 6,1462,053; 6,1388,077; 6,335,345; and 6,326,375; US Pat. Appl. Publ. Nos. 2002/0151456 and 20031036652; and PCT Publication Nos. WO 031010175, WO 03/082190 and receptor agonists or antagonists, orexin receptor antagonists, glucagon-like peptide-1 receptor agonists, ciliary neurotrophic factors, human agouti-related proteins (AGRP), ghrelin receptor antagonists, histamine 3 receptor antagonists or inverse agonists, neuromedin U receptor agonists and the like. Other anti-obesity agents are readily apparent to one of ordinary skill in the art.

Representative methods for using PDE10 inhibitors for the reduction of body fat or body weight, as well as the treatment or prevention of obesity, type 2 diabetes (non-insulin dependent diabetes), metabolic syndrome, glucose intolerance, and related health risks, symptoms are reported in WO 2005/120514.

The present invention also includes method of treating pain conditions and disorders. Examples of such pain conditions and disorders include, but are not limited to, inflammatory pain, hyperalgesia, inflammatory hyperalgesia, migraine, cancer pain, osteoarthritis pain, post-surgical pain, non-inflammatory pain, neuropathic pain, sub-categories of neuropathic pain including peripheral neuropathic pain syndromes, chemotherapy-induced neuropathy, complex regional pain syndrome, HIV sensory neuropathy, neuropathy secondary to tumor infiltration, painful diabetic neuropathy, phantom limb pain, postherpetic neuralgia, postmastectomy pain, trigeminal neuralgia, central neuropathic pain syndromes, central poststroke pain, multiple sclerosis pain, Parkinson disease pain, and spinal cord injury pain.

In a further embodiment compounds of the present invention are administered in combination with one or more other agents effective for treating pain. Such agents include analgesics, non-steroidal anti-inflammatory drugs (NSAIDs), opiods and antidepressants. In various embodiments, one or more agents are selected from the group consisting of buprenorphine, naloxone, methadone, levomethadyl acetate, L-alpha acetylmethadol (LAAM), hydroxyzine, diphenoxylate, atropine, chlordiazepoxide, carbamazepine, mianserin, benzodiazepine, phenoziazine, disulfuram, acamprosate, topiramate, ondansetron, sertraline, bupropion, amantadine, amiloride, isradipine, tiagabine, baclofen, propranolol, tricyclic antidepressants, desipramine, carbamazepine, valproate, lamotrigine, doxepin, fluoxetine, imipramine, moclobemide, nortriptyline, paroxetine, sertraline, tryptophan, venlafaxine, trazodone, quetiapine, zolpidem, zopiclone, zaleplon, gabapentin, memantine, pregabalin, cannabinoids, tramadol, duloxetine, milnacipran, naltrexone, paracetamol, metoclopramide, loperamide, clonidine, lofexidine, and diazepam.

The present invention also includes methods of treating schizophrenia and other psychotic disorders, as described above, with a combination of compounds of the present invention with one or more antipsychotic agents. Examples of suitable antipsychotic agents for use in combination with the compounds of the present invention include, but are not limited to, the phenothiazine (chlorpromazine, mesoridazine, thioridazine, acetophenazine, fluphenazine, perphenazine and trifluoperazine), thioxanthine (chlorprothixene, thiothixene), heterocyclic dibenzazepine (clozapine, olanzepine and aripiprazole), butyrophenone (haloperidol), dipheyylbutylpiperidine (pimozide) and indolone (molindolone) classes of antipsychotic agents. Other antipsychotic agents with potential therapeutic value in combination with the compounds in the present invention include loxapine, sulpiride and risperidone.

The present invention further includes methods of treating depression or treatment-resistant depression with a combination of compounds of the present invention with one or more antidepressants. Examples of suitable anti-depressants for use in combination with the compounds of the present invention include, but are not limited to, norepinephrine reuptake inhibitors (tertiary and secondary amine tricyclics), selective serotonin reuptake inhibitors (SSRIs) (e.g., fluoxetine, fluvoxamine, paroxetine and sertraline), monoamine oxidase inhibitors (MAOIs) (isocarboxazid, phenelzine, tranylcypromine, selegiline), reversible inhibitors of monoamine oxidase (RIMAs) (moclobemide), serotonin and norepinephrine reuptake inhibitors (SNRIs) (venlafaxine), corticotropin releasing factor (CRF) receptor antagonists, alpha-adrenoreceptor antagonists, and atypical antidepressants (bupropion, lithium, nefazodone, trazodone and viloxazine).

An effective dose of the compounds according to the invention, or their salts, is used, in addition to physiologically acceptable carriers, diluents and/or adjuvants for producing a pharmaceutical composition. The dose of the active compounds can vary depending on the route of administration, the age and weight of the patient, the nature and severity of the diseases to be treated, and similar factors. The daily dose can be given as a single dose, which is to be administered once, or be subdivided into two or more daily doses, and is as a rule 0.001-2000 mg. Particular preference is given to administering daily doses of 0.1-500 mg, e.g. 0.1-100 mg.

Suitable administration forms are oral, parenteral, intravenous, transdermal, topical, inhalative, intranasal and sublingual preparations. Particular preference is given to using oral, parenteral, e.g. intravenous or intramuscular, intranasal preparations, e.g. dry powder or sublingual, of the compounds according to the invention. The customary galenic preparation forms, such as tablets, sugar-coated tablets, capsules, dispersible powders, granulates, aqueous solutions, alcohol-containing aqueous solutions, aqueous or oily suspensions, syrups, juices or drops, are used.

Solid medicinal forms can comprise inert components and carrier substances, such as calcium carbonate, calcium phosphate, sodium phosphate, lactose, starch, mannitol, alginates, gelatine, guar gum, magnesium stearate, aluminium stearate, methyl cellulose, talc, highly dispersed silicic acids, silicone oil, higher molecular weight fatty acids, (such as stearic acid), gelatine, agar agar or vegetable or animal fats and oils, or solid high molecular weight polymers (such as polyethylene glycol); preparations which are suitable for oral administration can comprise additional flavourings and/or sweetening agents, if desired.

Liquid medicinal forms can be sterilized and/or, where appropriate, comprise auxiliary substances, such as preservatives, stabilizers, wetting agents, penetrating agents, emulsifiers, spreading agents, solubilizers, salts, sugars or sugar alcohols for regulating the osmotic pressure or for buffering, and/or viscosity regulators.

Examples of such additives are tartrate and citrate buffers, ethanol and sequestering agents (such as ethylenediaminetetraacetic acid and its non-toxic salts). High molecular weight polymers, such as liquid polyethylene oxides, microcrystalline celluloses, carboxymethyl celluloses, polyvinylpyrrolidones, dextrans or gelatine, are suitable for regulating the viscosity. Examples of solid carrier substances are starch, lactose, mannitol, methyl cellulose, talc, highly dispersed silicic acids, high molecular weight fatty acids (such as stearic acid), gelatine, agar agar, calcium phosphate, magnesium stearate, animal and vegetable fats, and solid high molecular weight polymers, such as polyethylene glycol.

Oily suspensions for parenteral or topical applications can be vegetable synthetic or semisynthetic oils, such as liquid fatty acid esters having in each case from 8 to 22 C atoms in the fatty acid chains, for example palmitic acid, lauric acid, tridecanoic acid, margaric acid, stearic acid, arachidic acid, myristic acid, behenic acid, pentadecanoic acid, linoleic acid, elaidic acid, brasidic acid, erucic acid or oleic acid, which are esterified with monohydric to trihydric alcohols having from 1 to 6 C atoms, such as methanol, ethanol, propanol, butanol, pentanol or their isomers, glycol or glycerol. Examples of such fatty acid esters are commercially available miglyols, isopropyl myristate, isopropyl palmitate, isopropyl stearate, PEG 6-capric acid, caprylic/capric acid esters of saturated fatty alcohols, polyoxyethylene glycerol trioleates, ethyl oleate, waxy fatty acid esters, such as artificial ducktail gland fat, coconut fatty acid isopropyl ester, oleyl oleate, decyl oleate, ethyl lactate, dibutyl phthalate, diisopropyl adipate, polyol fatty acid esters, inter alia. Silicone oils of differing viscosity, or fatty alcohols, such as isotridecyl alcohol, 2-octyldodecanol, cetylstearyl alcohol or oleyl alcohol, or fatty acids, such as oleic acid, are also suitable. It is furthermore possible to use vegetable oils, such as castor oil, almond oil, olive oil, sesame oil, cotton seed oil, groundnut oil or soybean oil.

Suitable solvents, gelatinizing agents and solubilizers are water or water-miscible solvents. Examples of suitable substances are alcohols, such as ethanol or isopropyl alcohol, benzyl alcohol, 2-octyldodecanol, polyethylene glycols, phthalates, adipates, propylene glycol, glycerol, di- or tripropylene glycol, waxes, methyl cellosolve, cellosolve, esters, morpholines, dioxane, dimethyl sulphoxide, dimethylformamide, tetrahydrofuran, cyclohexanone, etc.

Cellulose ethers which can dissolve or swell both in water or in organic solvents, such as hydroxypropylmethyl cellulose, methyl cellulose or ethyl cellulose, or soluble starches, can be used as film-forming agents.

Mixtures of gelatinizing agents and film-forming agents are also perfectly possible. In this case, use is made, in particular, of ionic macromolecules such as sodium carboxymethyl cellulose, polyacrylic acid, polymethacrylic acid and their salts, sodium amylopectin semiglycolate, alginic acid or propylene glycol alginate as the sodium salt, gum arabic, xanthan gum, guar gum or carrageenan. The following can be used as additional formulation aids: glycerol, paraffin of differing viscosity, triethanolamine, collagen, allantoin and novantisolic acid. Use of surfactants, emulsifiers or wetting agents, for example of Na lauryl sulphate, fatty alcohol ether sulphates, di-Na-N-lauryl-β-iminodipropionate, polyethoxylated castor oil or sorbitan monooleate, sorbitan monostearate, polysorbates (e.g. Tween), cetyl alcohol, lecithin, glycerol monostearate, polyoxyethylene stearate, alkylphenol polyglycol ethers, cetyltrimethylammonium chloride or mono-/dialkylpolyglycol ether orthophosphoric acid monoethanolamine salts can also be required for the formulation. Stabilizers, such as montmorillonites or colloidal silicic acids, for stabilizing emulsions or preventing the breakdown of active substances such as antioxidants, for example tocopherols or butylhydroxyanisole, or preservatives, such as p-hydroxybenzoic acid esters, can likewise be used for preparing the desired formulations.

Preparations for parenteral administration can be present in separate dose unit forms, such as ampoules or vials. Use is preferably made of solutions of the active compound, preferably aqueous solution and, in particular, isotonic solutions and also suspensions. These injection forms can be made available as ready-to-use preparations or only be prepared directly before use, by mixing the active compound, for example the lyophilisate, where appropriate containing other solid carrier substances, with the desired solvent or suspending agent.

Intranasal preparations can be present as aqueous or oily solutions or as aqueous or oily suspensions. They can also be present as lyophilisates which are prepared before use using the suitable solvent or suspending agent.

Inhalable preparations can present as powders, solutions or suspensions. Preferably, inhalable preparations are in the form of powders, e.g. as a mixture of the active ingredient with a suitable formulation aid such as lactose.

The preparations are produced, aliquoted and sealed under the customary antimicrobial and aseptic conditions.

As indicated above, the compounds of the invention may be administered as a combination therapy with further active agents, e.g. therapeutically active compounds useful in the treatment of central nervous system disorders. These further compounds may be PDE10 inhibitors or compounds which have an activity which is not based on PDE10 inhibition such as dopamine D2 receptor modulating agents or NMDA modulating agents.

For a combination therapy, the active ingredients may be formulated as compositions containing several active ingredients in a single dose form and/or as kits containing individual active ingredients in separate dose forms. The active ingredients used in combination therapy may be co-administered or administered separately.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) preventing the disease; for example, preventing a disease, condition or disorder in an individual who may be predisposed to the disease, condition or disorder but does not yet experience or display the pathology or symptomatology of the disease; (2) inhibiting the disease; for example, inhibiting a disease, condition or disorder in an individual who is experiencing or displaying the pathology or symptomatology of the disease, condition or disorder; and (3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

The synthesis of compounds of formula (IIa) preferably starts from imidazo[1,5-a]pyrido[3,2-e]pyrazinones of formula (III):

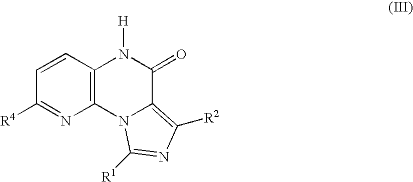

wherein R¹, R² and R⁴ are as described above.

The preparation of compounds of formula (III) is well described e.g. in WO 00/43392, WO 01/68097 and also by D. Norris et al. (Tetrahedron Letters 42 (2001), 4297-4299).

According to standard procedures known from the literature and already used in WO 99/45009 compounds of formula (III) are halogenated by treatment with halogenating reagents like POCl₃, PCl₃, PCl₅ SOCl₂, POBr₃, PBr₃ or PBr₅, yielding e.g. 4-chloro or 4-bromo-imidazo[1,5-a]pyrido[3,2-e]pyrazines of formula (IV):

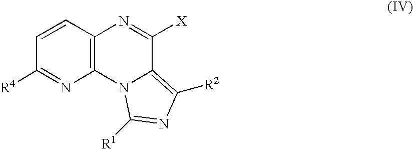

wherein X is Cl or Br and R¹, R² and R⁴ are as defined above.

Compounds of formula (IIa) where m and n are 0, the bond between A and N is a double bond and R³ is selected from OR⁶, SR⁶, OR⁷ or SR⁷ as described above, are preferably prepared by the treatment of an intermediate of formula (IV) with the corresponding alcohols or mercaptanes HOR⁶, HOR⁷, HSR⁶ or HSR⁷.

The synthesis of compounds of formula (IIb) and (IIc) starts from imidazo[1,5-a]pyrido[3,2-e]pyrazinones of formula (III),

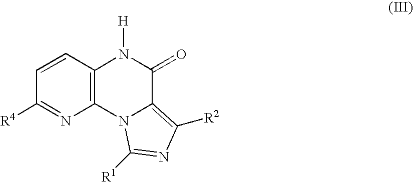

wherein R¹, R² and R⁴ are as described above.

The preparation of compounds of formula (III) is well described e.g. in WO 00/43392, WO 01/68097 and also by D. Norris et al. (Tetrahedron Letters 42 (2001), 4297-4299).

According to standard procedures known from the literature and already used in WO 99/45009 compounds of formula (III) are halogenated by treatment with halogenating reagents like POCl₃, PCl₃, PCl₅, SOCl₂, POBr₃, PBr₃ or PBr₅, yielding e.g. 4-chloro or 4-bromo-imidazo[1,5-a]pyrido[3,2-e]pyrazines of formula (IV),

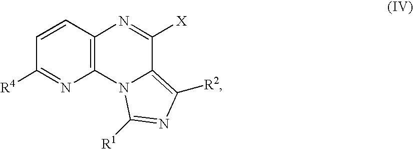

wherein X is Cl or Br, particularly Cl, and R¹, R² and R⁴ are as defined above.

Following this the chloro or bromo atom is substituted by amine treatment forming compounds of formula (IIb) and (IIc). Compounds of formula (IIb) and (IIc) with R⁵ and or R⁶ representing hydrogen can be transformed into N-acylated derivatives by the reaction with a very reactive carboxylic acid derivative. Carboxylic acid chlorides and anhydride are used preferentially.

### EXAMPLES

Examples 1a-94a relate to compounds of Formula (IIa)

Examples 1b-44b relate to compounds of Formula (IIb) and (IIc).

Compound names correspond to the following representative numbering system:

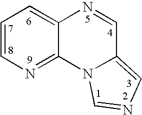

### Intermediate A1: 4-chloro-8-methoxy-3-methyl-1-propyl-imidazo[1,5-a]pyrido[3,2-e]pyrazine

16 g of 8-methoxy-3-methyl-1-propyl-imidazo[1,5-a]pyrido[3,2-e]pyrazine-4-one and 120 ml POCl₃ are mixed and heated up to reflux for 8 hours. After cooling to room temperature the reaction mixture is treated with 1200 ml crushed ice/water and stirred for 1 hour. The product is extracted with 2×300 ml dichloromethane. The collected organic layer is washed with 2×300 ml water and dried with Na₂SO₄. The solvent is removed under reduced pressure.

Yield: 14.5 g

m.p.: 121-123° C.

Many other intermediates A of formula (IV) can be prepared according to this procedure. Some examples are the following:

| | |
|---|---|
| | (IV) |
| 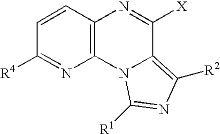 | |

| Intermediate | X | R¹ | R² | R⁴ | m.p.[° C.] |
|---|---|---|---|---|---|
| A1 | —Cl | —C₃H₇ | —CH₃ | —OCH₃ | 121-123 |
| A2 | —Cl | —C₂H₅ | —CH₃ | —OCH₃ | 148-150 |
| A3 | —Cl | —CH₃ | —CH₃ | —OCH₃ | 176-178 |
| A4 | —Cl | —C₆H₁₁ | —CH₃ | —OCH₃ | 211-213 |
| A5 | —Cl | —C₆H₁₃ | —CH₃ | —OCH₃ | 115-117 |
| A6 | —Cl | —C₅H₁₁ | —CH₃ | —OCH₃ | 110.5-113 |
| A7 | —Cl | —CH₂CH₂CF₃ | —CH₃ | —OCH₃ | 149-153 |
| A8 | —Cl | —(CH₂)₂C₆H₅ | —CH₃ | —OCH₃ | 130 |
| A9 | —Cl | —C₆H₅ | —CH₃ | —OCH₃ | 240-242 |
| A10 | —Cl | —C₆H₄(4-F) | —CH₃ | —OCH₃ | 256-258 |
| A11 | —Cl | —C₂H₅ | —CH₃ | —H | 117-120 |
| A12 | —Cl | —C₃H₇ | —CH₃ | —H | 138-140 |
| A13 | —Cl | —C₃H₇ | —H | —OCH₃ | 153-155 |
| A14 | —Cl | —CH(CH₃)₂ | —H | —OCH₃ | 162-164 |
| A15 | —Cl | —CH₃ | —H | —OCH₃ | 225-228 |
| A16 | —Cl | —H | —H | —H | 222-225 |
| A17 | —Cl | —H | —C₆H₅ | —OCH₃ | 168-171 |
| A18 | —Cl | —H | —CH₃ | —OCH₃ | 185-187 |
| A19 | —Cl | —C₃H₇ | —CH₃ | —CH₃ | 99-101 |
| A20 | —Cl | —C₂H₅ | —CH₃ | —N(C₂H₅)₂ | 145-150 |
| A21 | —Cl | —C₃H₇ | —CH₃ | 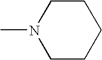 | |
| A22 | —Cl | —C₂H₅ | —CH₃ | 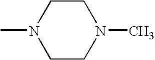 | 283-285 |
| A23 | —Cl | —C₂H₅ | —CH₃ | 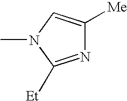 | 138-141 |
| A24 | —Cl | —C₃H₇ | —CH₃ | 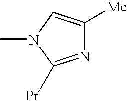 | 134-136 |

### Intermediate A25: 4-chloro-3-methyl-1-propyl-imidazo[1,5-a]pyrido[3,2-e]pyrazin-8-ol

2 g 4-chloro-8-methoxy-3-methyl-1-propyl-imidazo[1,5-a]pyrido[3,2-e]-pyrazine (Intermediate A1) was suspended in 50 ml dichloromethane. At 0-5° C. 3 ml bortribromide was added dropwise, followed by 1 h stirring at 0-5° C., 4 h stirring at room temperature, and standing over night. The reaction mixture was added slowly to a solution of 10 g potassium carbonate in 100 ml water. After stirring and constant pH>7 (adding 10% potassium carbonate solution) the precipitate was filtered off, and washed with water.

Yield: 1.87 g

m.p.: 227-234° C. (EtOH)

Other intermediates A of formula (IV) can be prepared according to this procedure. Examples with X=Br were obtained with a period of 6 h heating to reflux. Some examples are the following:

| Intermediate | X | R¹ | R² | R⁴ | m.p. [° C.] |
|---|---|---|---|---|---|
| A25 | —Cl | —C₃H₇ | —CH₃ | —OH | 227-234 |
| A26 | —Br | —C₂H₅ | —CH₃ | —OH | >360° C. |
| | | | | | (xHBr) |
| A27 | —Br | —C₆H₁₁ | —CH₃ | —OH | 212-216 |

### Intermediate A28: 4-chloro-8-difluoromethoxy-3-methyl-1-propyl-imidazo[1,5-a]pyrido[3,2-e]pyrazine

5.51 g (0.02 mol) 4-chloro-3-methyl-1-propyl-9H-imidazo[1,5-a]pyrido[3,2-e]pyrazin-8-ol (Intermediate A25) and 2 g (0.05 mol) sodium hydroxide were dissolved in 20 ml dimethylformamide. After 10 min stirring 2.53 ml (0.03 mol) chlorodifluoroacetic acid was added dropwise. The mixture was heated 5 h at 150° C. bath temperature with stirring. After cooling the product was extracted with ethyl acetate (200 ml, 300 ml), the combined organic phases were washed with water (2×100 ml), the organic phase was dried over sodium sulfate, filtered off, and evaporated to dryness.

The obtained residue with 3 alkylated products was separated by preparative chromatography (silica gel, dichloromethane/methanol=9/1, v/v).

Yield: 1.21 g

m.p.: 95-98° C.

### Example 1a

### 4,8-dimethoxy-3-methyl-1-propyl-imidazo[1,5-a]pyrido[3,2-e]pyrazine

1.5 g of intermediate A1 are dissolved in a mixture of 15 ml methanol and 15 ml dichloromethane. 1 g of solid KOH is added. The mixture is heated up to reflux for 7 hours. At room temperature 30 ml water are added. The organic layer is separated. The aqueous layer is extracted with 20 ml dichloromethane. The unified organic layers are washed with 2×20 ml water. The solvent is removed completely. The residue is purified by LC.

Yield: 1.2 g

m.p.: 112-115° C.

The following examples are prepared using the same route of synthesis and reaction conditions like described above for example 1a:

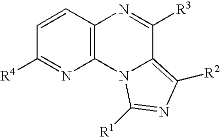

| Example | R¹ | R² | R³ | R⁴ | m.p. [° C.] |
|---|---|---|---|---|---|
| 1a | —C₃H₇ | —CH₃ | —OCH₃ | —OCH₃ | 112-115 |
| 2a | —C₃H₇ | —H | —OCH₃ | —OCH₃ | 113-116 |
| 3a | —C₂H₅ | —CH₃ | —OCH₃ | —OCH₃ | 155-157 |
| 4a | —CH₃ | —CH₃ | —OCH₃ | —OCH₃ | 184-186 |
| 5a | —H | —CH₃ | —OCH₃ | —OCH₃ | 152-154 |
| 6a | —C₂H₅ | —CH₃ | —OCH(CH₃)₂ | —OCH₃ | 80-81 |
| 7a | —C₂H₅ | —CH₃ | —OC₃H₇ | —OCH₃ | 78-81 |
| 8a | —C₂H₅ | —CH₃ | 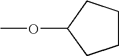 | —OCH₃ | 76-78 |
| 9a | —C₃H₇ | —CH₃ | —OCH(CH₃)₂ | —OCH₃ | 78-80 |
| 10a | —CH₃ | —CH₃ | 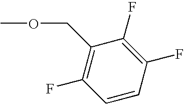 | —OCH₃ | 227-229 |
| 11a | —C₂H₅ | —CH₃ | 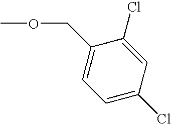 | —OCH₃ | 193-195 |
| 12a | —C₂H₅ | —CH₃ | 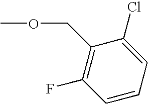 | —OCH₃ | 149-151 |
| 13a | —C₂H₅ | —CH₃ | 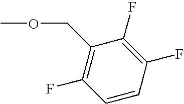 | —OCH₃ | 158-160 |
| 14a | —C₂H₅ | —CH₃ | 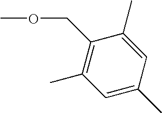 | —OCH₃ | 157-160 |
| 15a | —C₃H₇ | —CH₃ | 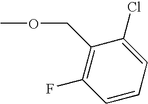 | —OCH₃ | 163-165 |
| 16a | —C₃H₇ | —CH₃ | 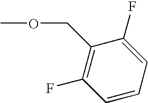 | —OCH₃ | 147-149 |
| 17a | —C₂H₅ | —CH₃ | 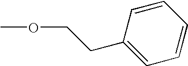 | —OCH₃ | 133-135 |
| 18a | —C₃H₇ | —CH₃ | 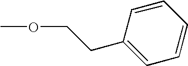 | —OCH₃ | 129-132 |
| 19a | —CH₃ | —CH₃ | 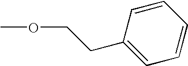 | —OCH₃ | 115-118 |
| 20a | —H | —CH₃ | 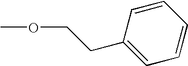 | —OCH₃ | 111-114 |
| 21a | —C₃H₇ | —CH₃ | 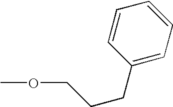 | —OCH₃ | 87-89 |
| 22a | —C₂H₅ | —CH₃ | 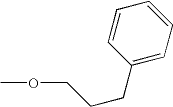 | —OCH₃ | 75-78 |
| 23a | —CH₃ | —CH₃ | 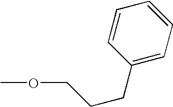 | —OCH₃ | 83-85 |
| 24a | —C₂H₅ | —CH₃ | 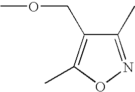 | —OCH₃ | 173-175 |
| 25a | —C₂H₅ | —CH₃ | —SCH₃ | —OCH₃ | 156-159 |
| 26a | —C₃H₇ | —CH₃ | —SCH₃ | —OCH₃ | 112-115 |
| 27a | —CH₃ | —CH₃ | —SCH₃ | —OCH₃ | 140-144 |
| 28a | —H | —CH₃ | —SCH₃ | —OCH₃ | 185-187 |

Compounds of formula (IIa) where m and n are 0, the bond between A and N is a double bond and R³ is —CN are preferably prepared by the treatment of an intermediate of formula (IV) with the Grignard reagent ethoxycarbonyl-difluoromethyl magnesium chloride followed by the substitution with a cyanide salt, e.g. KCN.

### Example 29a

### 4-cyano-8-methoxy-3-methyl-1-propyl-imidazo[1,5-a]pyrido[3,2-e]pyrazine

3 g of intermediate A1 are added into a solution of 32 g ethoxycarbonyl-difluoromethyl magnesia chloride in 100 ml tetrahydrofurane (THF). The mixture is stirred and heated up to reflux for 10 hours. Then the solvent is removed and 15 ml N,N-dimethylformamide and 2 g KCN are added. This reaction mixture is heated up to reflux for 5 hours. After this time 100 ml toluol are added. The organic layer is washed with 3×50 ml water. The solvent is removed and purified by preparative HPLC.

Yield: 0.2 g

m.p.: 178-180° C.

Using the same procedure and reaction conditions like described above for Example 29a also Example 30a was synthesized.

### Example 30a

### 4-cyano-8-methoxy-3-methyl-1-ethyl-imidazo[1,5-a]pyrido[3,2-e]pyrazine

Yield: 0.14 g

m.p.: 171-178° C.

Compounds of formula (IIa) where m and n are 0, the bond between A and N is a double bond and R³ is —N₃ are prepared by the treatment of an intermediate of formula (IV) with and an azide salt, e.g. NaN₃.

### Example 31a

### 4-azido-8-methoxy-3-methyl-1-propyl-imidazo[1,5-a]pyrido[3,2-e]pyrazine

1.5 g of intermediate A1 are stirred into 10 ml N,N-dimethylformamide. 1 g NaN₃ is added at room temperature. The mixture is heated up to 60° C. and stirred for 5 hours. 100 ml toluol are added. The organic layer is separated and washed with 3×30 ml water. 90 ml of the solvent are removed. The reaction product precipitates. The crude product is purified by crystallisation from toluol.

Yield: 1.2 g

m.p.: >205° C. (decomp.)

Compounds of formula (IIa) where m and n are 0, the bond between A and N is a double bond and R³ is (SO)R⁶ or (SO₂)R⁶, wherein R⁶ is as defined above, are prepared by oxidation of the corresponding compounds of formula (II) where R³ means —SR⁶.

### Example 32a

### 8-methoxy-3-methyl-4-methylsulfinyl-1-propyl-imidazo[1,5-a]pyrido[3,2-e]pyrazine and

### Example 33a

### 8-methoxy-3-methyl-4-methylsulfonyl-1-propyl-imidazo[1,5-a]pyrido[3,2-e]pyrazine

0.7 g of 8-methoxy-3-methyl-4-methylthio-1-propyl-imidazo[1,5-a]pyrido[3,2-e]pyrazine (Example 26a) are dissolved in 40 ml dichloromethane. 0.8 g of 3-chloroperoxybenzoic acid are added at 0 to 5° C. in small portions. The mixture is stirred for 2 hours at room temperature. The solution is washed with 2×30 ml saturated NaHCO₃ solution and than with 2×30 ml water. The solvent is removed from the isolated organic layer. The crude mixture of Example 32 and Example 33 is separated by preparative HPLC.

### Example 32a

Yield: 0.2 g

m.p.: 144-147° C.

### Example 33a

Yield: 0.25 g

m.p.: 42-46° C.

Example 34a is prepared using the same route of synthesis and reaction conditions like described above for example 31a:

### Example 34a

### 1-ethyl-8-methoxy-3-methyl-4-methylsulfinyl-imidazo[1,5-a]pyrido[3,2-e]pyrazine

Yield: 0.23 g

m.p.: 189-192° C.

Compounds of formula (IIa) where m and n are 0, the bond between A and N is a double bond and R³ is hydrogen are preferably prepared by the hydrogenation of an intermediate of formula (IV), e.g. with hydrogen in the presence of a catalyst such as palladium.

### Example 35a

### 8-methoxy-3-methyl-1-propyl-imidazo[1,5-a]pyrido[3,2-e]pyrazine

2 g of intermediate A1 are suspended in 50 ml ethanol. 1 ml triethylamine and 1 g palladium catalyst are added. An autoclave is used as reaction vessel. Hydrogen is pressed in up to 20 bar pressure. Now, the mixture is stirred at 30° C. for 4 hours. After filtration the solvent is removed. The crude product is dissolved in 100 ml dichloromethane. This solution is washed with 50 ml water. The solvent is removed to isolate pure product.

Yield: 1.3 g

m.p.: 134-135° C.

Using the same procedure and reaction conditions like described above for Example 35a also Example 36a was synthesized.

### Example 36a

### 1-ethyl-8-methoxy-3-methyl-imidazo[1,5-a]pyrido[3,2-e]pyrazine

Yield: 1.0 g

m.p.: 159-162° C.

Compounds of formula (IIa) where m and n are 0, the bond between A and N is a double bond and R³ is R⁶ as described above, are preferably prepared by treatment of an intermediate of formula (IVa) with the corresponding alkyl-, alkenyl- or alkynyl organometal reagent, e.g. ethyl magnesium bromide.

### Example 37a

### 4-ethyl-8-methoxy-3-methyl-1-propyl-imidazo[1,5-a]pyrido[3,2-e]pyrazine

7 g of intermediate A1 are suspended in 150 ml tetrahydrofurane. 30 ml of a solution of ethyl magnesium bromide in tetrahydrofurane (3 M) are added. The mixture is stirred for 4 hours at room temperature. After filtration the solvent is removed. The crude product is purified by preparative HPLC.

Yield: 5.1 g

m.p.: 78-81° C.

The following compounds are prepared using the same route of synthesis and reaction conditions like described above for Example 37a:

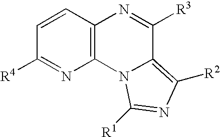

| Example | R¹ | R² | R³ | R⁴ | m.p. [° C.] |
|---|---|---|---|---|---|
| 37a | —C₃H₇ | —CH₃ | —C₂H₅ | —OCH₃ | 78-81 |
| 38a | —C₃H₇ | —CH₃ | —CH₃ | —OCH₃ | 91-93 |
| 39a | —C₃H₇ | —CH₃ | —CH₃ | —OCH₃ | 171-175 |
| | | | | | (×HCl) |
| 40a | —C₂H₅ | —CH₃ | —CH₃ | —OCH₃ | 106-109 |
| 41a | —CH₃ | —CH₃ | —CH₃ | —OCH₃ | 157-161 |
| 42a | —CH₂CH₂CF₃ | —CH₃ | —CH₃ | —OCH₃ | 145-147 |
| 43a | —C₅H₁₁ | —CH₃ | —CH₃ | —OCH₃ | 70-71 |
| 44a | —C₆H₁₁ | —CH₃ | —CH₃ | —OCH₃ | 149-152 |
| 45a | —C₆H₁₃ | —CH₃ | —CH₃ | —OCH₃ | 73-75 |
| 46a | —(CH₂)₂C₆H₅ | —CH₃ | —CH₃ | —OCH₃ | 121.5-123 |
| 47a | —C₆H₅ | —CH₃ | —CH₃ | —OCH₃ | 189-192 |
| 48a | —C₆H₅ | —CH₃ | —CH₃ | —OCH₃ | 210-218 |
| | | | | | (×2 HCl) |
| 49a | —C₆H₄(2-Cl) | —CH₃ | —CH₃ | —CH₃ | 220-222 |
| 50a | —C₆H₄(4-F) | —CH₃ | —CH₃ | —OCH₃ | 235-238 |
| 51a | —C₃H₇ | —CH₃ | —CH₃ | —CH₃ | 104-107 |
| 52a | —C₃H₇ | —CH₃ | —CH₃ | —H | 92-95 |
| 53a | —C₃H₇ | H | —CH₃ | —OCH₃ | 124-126 |
| 54a | —C₃H₇ | —CH₃ | —CH₃ | —OCHF₂ | 126-130 |
| 55a | —C₃H₇ | —CH₃ | —CH₃ | 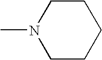 | 98-101 |
| 56a | —C₂H₅ | —CH₃ | —CH₃ | 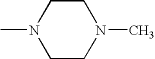 | 146-149 |
| 57a | —C₂H₅ | —CH₃ | —CH₃ | 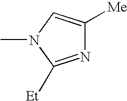 | 73-75 |
| 58a | —C₃H₇ | —CH₃ | —CH₃ | 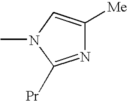 | 105-107 |

### Example 38a

### 3,4-Dimethyl-8-methoxy-1-propyl-imidazo[1,5-a]-pyrido[3,2-e]-pyrazine (alternatively 2-methoxy-6,7-dimethyl-9-propylimidazo[1,5-a]pyrido[3,2-e]pyrazine)

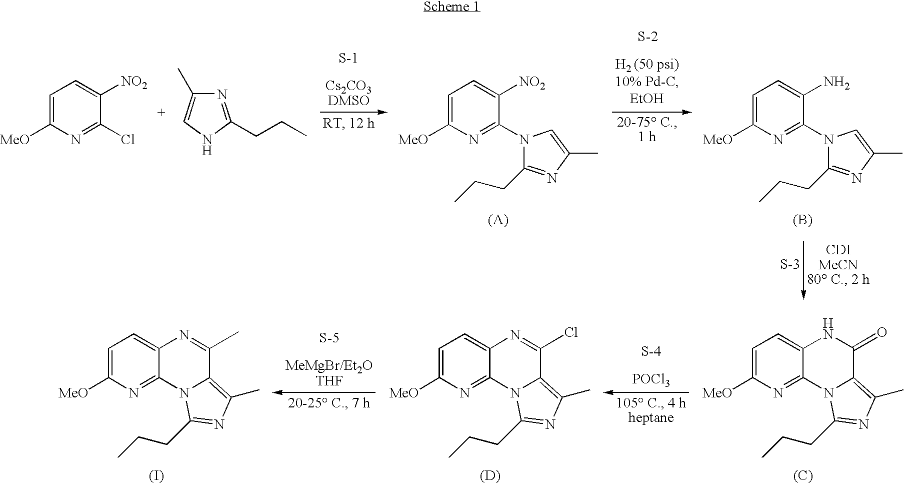

Compound (A) was obtained (yield about 90%, purity about 95-96%) by coupling of 2-chloro-6-methoxy-3-nitropyridine and 4-methyl-2-propyl-1H-imidazole in an organic solvent (e.g., DMSO or NMP) at room temperature in the presence of a base (e.g., cesium carbonate).

Compound (A) was subsequently reduced under a catalytic hydrogenation condition (e.g., 10% Pd—C (50% wet) and hydrogen (50 psi)). Treatment of compound (B) with 1,1′-carbonyldiimidazole in acetonitrile at about 80° C. yielded compound (C) in a high yield (about 96%) and high purity (about 99%), which then reacted with POCl3 to gave compound (D) (Yield ˜95%; Purity ˜98%). The conversion of compound (D) to compound (I) was achieved by treatment of compound (D) with a Grignard reagent (e.g., methyl magnesiumbromide).

Alternatively, 3,4-Dimethyl-8-methoxy-1-propyl-imidazo[1,5-a]-pyrido[3,2-e]-pyrazine (2-methoxy-6,7-dimethyl-9-propylimidazo[1,5-a]pyrido[3,2-e]pyrazine) free base can be prepared according to Scheme 2.

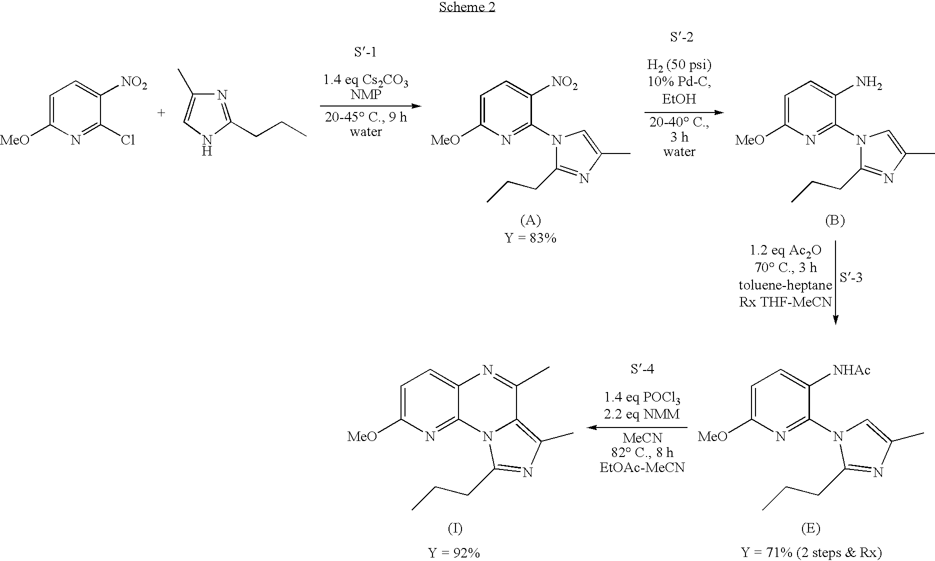

Coupling of 4-methyl-2-propyl-1H-imidazole and 2-chloro-6-methoxy-3-nitropyridine in the presence of cesium carbonate provided compound (A). Compound (A) was then reduced to compound (B) under a catalytic hydrogenation reaction condition (e.g., hydrogen (50 psi), 10% Pd—C). The hydrogenation reaction was carried out in an alcohol solvent (e.g., ethanol) at an elevated temperature (e.g., about 20-40° C.).

Acetylation of compound (B) by acetic anhydride at an elevated temperature (e.g., about 70° C.) yielded compound (E), which was purified by crystallization from acetonitrile and tetrahydrofuran. The crystallization was achieved by dissolving compound (E) in THF at reflux, removing solvent under reduced pressure until crystallization occurred, and adding anti-solvent (e.g., acetonitrile).

Compound (E) subsequently reacted with POCl3 (about 1.4 eq.) in refluxing acetonitrile (80-85° C.) in the presence of a base (e.g., N-methylmorpholine) (2.2 eq.).

Crystalline hemi-succinic salt of 2-methoxy-6,7-dimethyl-9-propylimidazo[1,5-a]pyrido[3,2-e]pyrazine was generated by dissolving 100 mg of freebase in 0.4 mL ethanol at 20-30° C. To this solution, 44 mg of succinic acid dissolved in 0.25 mL ethanol was added. To this solution, 0.4 mL of heptane was added over 5 min. The resulting suspension was stirred at room temperature for 30 minutes and filtered, washed with 0.1 mL ethanol and dried at 40° C. in a vacuum oven to afford 92 mg of white solid (98.7% by NMR). The salt was characterized by polarized optical microscopy to show birefringence with extinction supporting that the sample was crystalline. Powder x-ray diffraction confirmed crystallinity. An endothermic melt event was observed at 132° C. using differential scanning calorimetry measurement. Thermogravimetry analysis indicated no significant weight loss until the melting point. Proton NMR measurement indicated a 1:1 acid/base stoichiometry. Further analysis of the data indicated that the NMR stoichiometry could have resulted from excess succinic acid present in the sample. The XRPD pattern matched that of the sample generated by adding 0.5 eq. succinic acid.

An analogous compound with R³=CH₃ was obtained during the synthesis of the above described of intermediate A28. Separation of the obtained 3 alkylated products by preparative chromatography resulted in Example 59a.

### Example 59a

### 4-difluoromethoxy-3-methyl-1-propyl-imidazo[1,5-a]pyrido[3,2-e]pyrazine-8-ol

Yield: 0.81 g

m.p.: 292-297° C.

Compounds of formula (IIa) where m is 0, n=1 and the bond between A and N is a double bond are synthesized from compounds of formula (IIa) where m and n are 0, the bond between A and N is a double bond by oxidation, e.g. with 3-chloroperoxybenzoic acid.

### Example 60a

### 8-methoxy-3-methyl-5-oxo-1-propyl-imidazo[1,5-a]pyrido[3,2-e]pyrazine

6 g of 8-methoxy-3-methyl-1-propyl-imidazo[1,5-a]pyrido[3,2-e]pyrazine (Example 35a) are dissolved in 300 ml dichloromethane. A solution of 12 g 3-chloroperoxybenzoic acid in 40 ml acetic acid is added in small portions during 30 minutes. The reaction mixture is stirred for 16 hours at room temperature. Than the solution is washed with 2×50 ml saturated NaHCO₃ solution and with 50 ml water. The solvent is removed. The crude product is purified by preparative HPLC.

Yield: 1.5 g

m.p.: 228-232° C.

The same route of synthesis and reaction conditions like described above for Example 37a were used for the synthesis of Example 42a.

### Example 61a

### 3,4-dimethyl-8-methoxy-5-oxo-1-propyl-imidazo[1,5-a]pyrido[3,2-e]pyrazine

Yield: 1.4 g

m.p.: 154-157° C.

Compounds of formula (IIa) where m and n are 0, the bond between A and N is a double bond and R³ is NH(CO)OR⁶, N((CO)OR⁶)₂, N(R⁶)((CO)OR⁶), NH(CO)NH₂, NH(CO)NHR⁶, NR⁶(CO)NH₂ and NR⁶(CO)NHR⁶ are preferably prepared by treatment of an intermediate of formula (IV) with NH₃ or an alkyl amine, e.g. a C₁₋₅ alkyl amine to form the corresponding 4-amino derivatives (according to the method from WO 99/45009). These 4-amino derivatives (intermediates B) are treated with suitable reagents such as chloro formic acid esters or amides to prepare the final products.

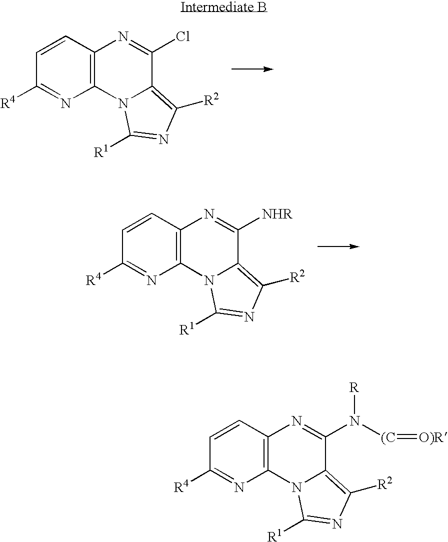

### Intermediate B1: 4-amino-8-methoxy-3-methyl-1-propyl-imidazo[1,5-a]pyrido[3,2-e]pyrazine

10 g of intermediate A1 and 200 ml of an aqueous solution of NH₃ (32%) are mixed in an autoclave and heated up to 130° C. for 8 hours. The reaction mixture is diluted with 200 ml water. The precipitated reaction product is separated washed with water and dichloro methane and dried at reduced pressure.

Yield: 8.5 g

m.p.: 219-221° C.

### Example 62a

### 8-methoxy-4-methoxycarbonylamino-3-methyl-1-propyl-imidazo[1,5-a]pyrido[3,2-e]pyrazine

1.4 g of the intermediate B1 are stirred with 20 ml dichloromethane 5 ml methanol and 1 ml triethylamine. At 0° C. a solution of 0.6 g chloro formic acid methylester in 10 ml dichloromethane is added slowly. The mixture is stirred for 2 hours at 0° C. Than the solution is heated up to reflux 10 hours. The solution is washed with 30 ml saturated NaHCO₃ solution and with 30 ml water. The solvent is removed. The crude product is purified by preparative HPLC.

Yield: 0.22 g

m.p.: 137-138° C.

Further Examples prepared using the same route of synthesis and reaction conditions like described above for Example 62a are the following:

### Example 63a

### 4-ethoxycarbonylamino-8-methoxy-3-methyl-1-propyl-imidazo[1,5-a]pyrido[3,2-e]pyrazine

Yield: 0.3 g

m.p.: 122-124° C.

### Example 64a

### 4-(N,N-bis-methoxycarbonyl-)amino-8-methoxy-3-methyl-1-propyl-imidazo[1,5-a]pyrido[3,2-e]pyrazine

Yield: 0.45 g

m.p.: 137-138° C.

### Example 65a

### 8-methoxy-4-(methoxycarbonyl-methyl-amino)-3-methyl-1-propyl-imidazo[1,5-a]pyrido[3,2-e]pyrazine

Yield: 0.04 g

m.p.: 105-109° C.

### Example 66a

### 8-methoxy-3-methyl-4-(3-methyl-ureido)-1-propyl-imidazo[1,5-a]pyrido[3,2-e]pyrazine

543 mg of Intermediate B1 and 960 mg N,N′-carbonyldiimidazole were stirred with 20 ml tetrahydrofurane for 3 hours under reflux. At room temperature 3 ml 40% methylamine solution was added slowly. The solution was heated up to reflux 30 minutes. After removing the solvent under reduced pressure the residue was extracted with 50 ml dichloromethane and 2×25 ml water. The organic layer is removed. The crude product was purified by preparative HPLC.

Yield: 0.4 g

m.p.: 178-181° C.

Further Examples prepared using the same route of synthesis and reaction conditions like described above for Example 66 are the following:

### Example 67a

### 8-methoxy-3-methyl-1-propyl-4-ureido-imidazo[1,5-a]pyrido[3,2-e]pyrazine

Yield: 0.5 g

m.p.: 185-187° C.

### Example 68a

### 8-methoxy-3-methyl-4-(3-isopropyl-ureido)-1-propyl-imidazo[1,5-a]pyrido[3,2-e]pyrazine

Yield: 0.3 g

m.p.: 165-166° C.

Compounds of formula (IIa) where m and n are 0, the bond between A and N is a double bond and R³ is NH—SO₂R⁶, N(SO₂R⁶)₂, N(R⁶)(SO₂R⁶), NHSO₂R⁷, N(SO₂R⁷)₂ and N(R⁸)SO₂R⁷, wherein R⁶, R⁷ and R⁸ are as defined above, are preferably prepared by treatment of an intermediate of formula (IV) with NH₃ or an alkyl amine, e.g. a C₁₋₅ alkyl amine to form the corresponding 4-amino derivatives according to the method from WO 99/45009. These 4-amino derivatives (intermediates B) are treated with sulfonic acid chlorides or anhydrides forming the final sulfonamides.

### Example 69

### 8-methoxy-3-methyl-4-methylsulfonylamino-1-propyl-imidazo[1,5-a]pyrido[3,2-e]pyrazine

10 g of the intermediate B1 are mixed with 350 ml toluol and 14 g methylsulfonic acid anhydride. The mixture is heated up to reflux for 1 hour. After this time 16 ml triethylamine are added at 70° C. The mixture is stirred then for 1 hour. 100 ml water are added. The product precipitates. After filtration it is washed with 3×80 ml water and 3×80 ml toluol. The product is crystallized from toluol.

Yield: 9 g

m.p.: 243-246° C.

Further Examples prepared using the same route of synthesis and reaction conditions like described above for Example 46a are the following:

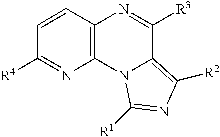

| Example | R¹ | R² | R³ | R⁴ | m.p. [° C.] |
|---|---|---|---|---|---|
| 69a | —C₃H₇ | —CH₃ | —NHSO₂CH₃ | —OCH₃ | 243-246 |
| 70a | —C₃H₇ | —CH₃ | —N(SO₂CH₃)₂ | —OCH₃ | 198-200 |
| 71a | —C₃H₇ | —CH₃ | —NHSO₂C₂H₅ | —OCH₃ | 189-190 |
| 72a | —C₂H₅ | —CH₃ | —NHSO₂CH₃ | —OCH₃ | 270-271 |
| 73a | —C₃H₇ | —CH₃ | —NHSO₂CF₃ | —OCH₃ | 213-216 |
| 74a | —C₃H₇ | —CH₃ | —NHSO₂C₃H₇ | —OCH₃ | 203-206 |
| 75a | —C₃H₇ | —CH₃ | —NHSO₂CH(CH₃)₂ | —OCH₃ | 235-238 |
| 76a | —C₃H₇ | —CH₃ | —NHSO₂(C₆H₄-4-CH₃) | —OCH₃ | 229-232 |
| 77a | —C₃H₇ | —CH₃ | —N[SO₂(C₆H₄-4-CH₃)]₂ | —OCH₃ | 206-209 |
| 78a | —(CH₂)₂CF₃ | —CH₃ | —NHSO₂CH₃ | —OCH₃ | 250-253 |
| 79a | —C₆H₁₃ | —CH₃ | —NHSO₂CH₃ | —OCH₃ | 134-136 |
| 80a | —(CH₂)₂C₆H₅ | —CH₃ | —NHSO₂CH₃ | —OCH₃ | 199-202 |
| 81a | —C₆H₅ | —CH₃ | —NHSO₂CH₃ | —OCH₃ | 217-220 |
| 82a | —C₆H₄(2-Cl) | —CH₃ | —NHSO₂CH₃ | —OCH₃ | 246-251 |
| 83a | —C₆H₄(4-F) | —CH₃ | —NHSO₂CH₃ | —OCH₃ | 250-256 |
| 84a | —C₃H₇ | —CH₃ | —NHSO₂CH₃ | 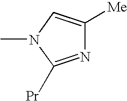 | 224-225 |

### Example 85a

### 3-methyl-4-methylsulfonylamino-1-propyl-imidazo[1,5-a]pyrido[3,2-e]pyrazin-8-ol hydrobromide

3 g 8-methoxy-3-methyl-4-methylsulfonylamino-1-propyl-imidazo[1,5-a]pyrido[3,2-e]pyrazine (Example 69) was suspended in 150 ml dichloromethane. At 0-5° C. 3.3 g bortribromide was added dropwise, followed by 30 min stirring at 0-5° C., 30 min stirring at room temperature, and 2 h at 30° C. The reaction mixture was added slowly to a solution of 10 g sodium carbonate in 100 ml water. After stirring and constant pH>7 (adding 10% potassium carbonate solution) the precipitate was filtered off, washed with water, dried, and recrystallized with ethanol.

Yield: 0.5 g

m.p.: 302-306° C.

### Example 86a

### 3-methyl-4-methylsulfonylamino-1-propyl-imidazo[1,5-a]pyrido[3,2-e]pyrazin-8-ol

Example 86a can be prepared according to procedure of Example 85 without 2 h stirring at 30° C.

Yield: 0.5 g

m.p.: 295-297° C.

### Example 87a

### 8-difluoromethoxy-3-methyl-4-methylsulfonylamino-1-propyl-imidazo[1,5-a]pyrido[3,2-e]pyrazine

4.98 g 3-methyl-4-methylsulfonylamino-1-propyl-imidazo[1,5-a]pyrido[3,2-e]pyrazin-8-ol (Example 86) and 1.6 g sodium hydroxide were dissolved in 20 ml dimethylformamide. After 10 min stirring 1.85 ml chlorodifluoroacetic acid was added dropwise. The mixture was heated 5 h at 150° C. bath temperature with stirring. After cooling the product was extracted with ethyl acetate (200 ml, 300 ml), the combined organic phases were washed with water (2×100 ml), the organic phase was dried over sodium sulfate, filtered off, and evaporated to dryness.

The obtained residue was separated by preparative chromatography (silica gel, dichloromethane/methanol=9/1, v/v).

Yield: 0.66 g

m.p.: 210-214° C.

### Example 88a

### 8-cyclopropylmethoxy-3-methyl-4-methylsulfonylamino-1-propyl-imidazo[1,5-a]pyrido[3,2-e]pyrazine

0.83 g 3-methyl-4-methylsulfonylamino-1-propyl-imidazo[1,5-a]pyrido[3,2-e]pyrazin-8-ol (Example 86) was dissolved in 20 ml dimethylformamide. 1.14 g cesium carbonate was added followed by 0.44 ml cyclopropyl bromide dropwise. The mixture was heated 1 h at 60° C. and 3 h at 130° C. bath temperature with stirring. After cooling the product was extracted with ethyl acetate (2×50 ml), and water (2×50 ml), the organic phase was dried over sodium sulfate, filtered off, and evaporated to dryness.

The obtained residue was separated by preparative chromatography (silica gel, dichloromethane/methanol=95/5, v/v).

Yield: 0.26 g

m.p.: 212-216° C.

Compounds of formula (IIa) where m=1, n is 0, the bond between A and N is a single bond and R⁵ is hydrogen are prepared by the reduction of an intermediate of formula (IV) with hydrogen, e.g. in the presence of a catalyst such as palladium.

### Example 89a

### 3-methyl-1-propyl-4,5-dihydro-imidazo[1,5-a]pyrido[3,2-e]pyrazine

6 g of the intermediate A12 are suspended in 200 ml ethanol. 3 ml triethylamine and 3 g palladium catalyst are added. An autoclave is used as reaction vessel. Hydrogen is pressed in up to 20 bar pressure. Now, the mixture is stirred at 70° C. for 4 hours. After filtration the solvent is removed. The crude product is dissolved in 100 ml dichloromethane. This solution is washed with 50 ml water. The solvent is removed to isolate the pure product.

Yield: 4.5 g

m.p.: 169-172° C.

Further Examples prepared using the same route of synthesis and reaction conditions like described above for Example 89a are the following:

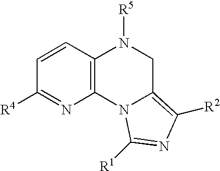

| Example | R¹ | R² | R⁴ | R⁵ | m.p. [° C.] |
|---|---|---|---|---|---|
| 89a | —C₃H₇ | —CH₃ | —H | —H | 169-172 |
| 90a | —C₃H₇ | —H | —OCH₃ | —H | 45-49 |
| 91a | —C₃H₇ | —CH₃ | —OCH₃ | —H | 157-160 |
| 92a | —C₃H₇ | —CH₃ | —OCH₃ | —H × HCl | 228-231 |
| 93a | —C₂H₅ | —CH₃ | —OCH₃ | —H | 139-142 |

Compounds of formula (IIa) where m=1, n is 0, the bond between A and N is a single bond and R⁵ is —C₁₋₅ alkyl are prepared by the treatment of compounds of formula (IIa) where m=1, n is 0, the bond between A and N is a single bond and R⁵ is hydrogen with a C₁₋₅ alkyl-aldehyde, e.g. in the presence of Raney-Nickel and hydrogen.

### Example 94a

### 3,5-dimethyl-8-methoxy-1-propyl-4,5-dihydro-imidazo[1,5-a]pyrido[3,2-e]pyrazine

1 g 8-methoxy-3-methyl-1-propyl-4,5-dihydro-imidazo[1,5-a]pyrido[3,2-e]pyrazine (Example 91) is suspended in 70 ml methanol. 1 ml methanal and 0.5 g Raney-Nickel are added. An autoclave is used as reaction vessel. Hydrogen is pressed in up to 20 bar pressure. Now, the mixture is stirred at 45° C. for 8 hours. After filtration the solvent is distilled off.

Yield: 0.97 g

m.p.: 113-116° C.

Compounds of formula (IIa) where m=1, n is 0, the bond between A and N is a single bond and R⁵ is —(C═O)—C₁₋₅ alkyl are prepared by treatment of compounds of formula (IIa) where m=1, n is 0, the bond between A and N is a single bond and R⁵ is hydrogen with alkyl acid chlorides or anhydrides.

### Example 95a

### 5-acetyl-8-methoxy-3-methyl-1-propyl-4,5-dihydro-imidazo[1,5-a]pyrido[3,2-e]pyrazine

1 g 8-methoxy-3-methyl-1-propyl-4,5-dihydro-imidazo[1,5-a]pyrido[3,2-e]pyrazine (Example 91a) is suspended in 25 ml dichloromethane. 0.8 g triethylamine are added. At 0° C. a solution of 0.4 g acetyl chloride in 5 ml dichloromethane is added.

The mixture is stirred for 2 hours at room temperature. 25 ml water are added. The organic layer is separated. The solvent is distilled off.

Yield: 1 g

m.p.: 114-116° C.

The Synthesis of the preferred compound (Example 38a/39a) is described in the following scheme over all steps:

### Step 1: 6-methoxy-2-(4-methyl-2-propyl-imidazol-1-yl)-3-nitro-pyridine

To a suspension prepared of 20.0 g KOH (solid), 25.8 g 4-methyl-2-propyl imidazole and 130 ml dimethyl formamide were added 38.0 g 2-chloro-6-methoxy-3-nitro pyridine in small amounts at a reaction temperature of 5° C. The reaction mixture was stirred for 75 minutes at room temperature. Then the reaction mixture was poured in 600 ml water. The mixture was further stirred for 1 hr. The desired product precipitated during this time. The resulting solid was collected by filtration, washed with 100 ml water for 3 times and dried in a dry box with vacuum (40° C.).

Yield: 40 g

m.p.: 96-103° C.

### Step 2: 3-amino-6-methoxy-2-(4-methyl-2-propyl-imidazol-1-yl)-pyridine

To a solution prepared of 138.2 g 6-methoxy-2-(4-methyl-2-propyl-imidazol-1-yl)-3-nitro-pyridine and 900 ml ethyl alcohol 4 g palladium-charcoal were added. The reaction mixture was heated to 40° C. and then hydrogenated under pressure (10 to 15 bar). At room temperature the catalyst was filtrated off and the filtrate was evaporated. To the solid residue 150 ml methyl tert.-butyl ether (MTBE) were added. After stirring for 30 minutes the product was collected by filtration, washed with 50 ml MTBE for 2 times and dried in a dry box with vacuum (40° C.).

Yield: 100 g

m.p.: 124-128° C.

### Step 3: 8-methoxy-3-methyl-1-propyl-imidazo[1,5-a]-pyrido[3,2-e]-pyrazinone

A mixture of 20 g 3-amino-6-methoxy-2-(4-methyl-2-propyl-imidazol-1-yl)-pyridine and 60 g urea were heated up to 160° C. The reaction mixture was stirred for 2 hrs. Then 10 ml of glacial acetic acid were added. The stirring was continued for further 6 hrs. The reaction mixture was allowed to cool. At a temperature of 70° C. 300 ml of water were added and the mixture was stirred for 1 hr at 50° C. The warm mixture was filtrated and washed with 50 ml of water for 2 times and dried in a dry box.

Yield: 20.5 g

m.p.: 297-300° C.

### Step 4: 4-chloro-8-methoxy-3-methyl-1-propyl-imidazo[1,5-a]-pyrido[3,2-e]-pyrazine

A mixture of 27 g 8-methoxy-3-methyl-1-propyl-imidazo[1,5-a]-pyrido[3,2-e]-pyrazinone and 225 ml phosphorus oxychloride were heated to reflux for 8 hrs. To the cooled mixture 250 ml of toluene were added and then 350 ml of the liquid were distilled off. Subsequently the same procedure was performed with 150 ml toluene but 250 ml of the liquid were distilled off. The reaction mixture was allowed to cool at room temperature and then poured in a mixture of 500 g ice/500 ml water. After 30 minutes the mixture was extracted with 250 ml of dichloromethane for two times. The dichloromethane layer was then washed with 500 ml water then with sodium carbonate (3% in water) and after that with 500 ml water. The organic layer was dried with sodium sulfate. After removal of the sodium sulfate and evaporation of the dichloromethane the crude product was dried in a dry box with vacuum (40° C.).

Yield: 26.5 g

m.p.: 119-123° C.

### Step 5: 3,4-Dimethyl-8-methoxy-1-propyl-imidazo[1,5-a]-pyrido[3,2-e]-pyrazine (Example 38a)

To a solution prepared of 20 g 4-chloro-8-methoxy-3-methyl-1-propyl-imidazo[1,5-a]-pyrido[3,2-e]-pyrazine (Intermediate 3) and 400 ml tetrahydrofuran 80 ml methylmagnesium bromide (3 M in diethyl ether) were added drop wise (via 2 hrs). The reaction mixture was stirred at room temperature for 6 hours. After that the mixture was poured in a mixture of 300 g water, which contained 100 g of ice and 10 g of ammonium chloride. The mixture was extracted for 4 times with 300 ml dichloromethane. The organic layer was separated and then dried with sodium sulfate. After removal of the sodium sulfate and evaporation of the dichloromethane a yellowish-orange crude product remained. This residue was stirred in 150 ml of diethyl ether. After 1 hr. the product was filtrated off and dried in a dry box.

The yield was 11.9 g of crude product (content>95%).

To a solution of 0.05 mol of the crude product and 100 ml of dichloromethane 2.5 equiv. of hydrochloric acid dissolved in 100 ml of water were added. The mixture was vigorously stirred. The dichloromethane layer was then separated and subsequently the water layer was extracted for 6 times with 100 ml dichloromethane. To the organic layer 15 g of sodium carbonate were added. After filtration of the solid precipitate and evaporation of the dichloromethane yellowish crystals remains.

Yield: 18.6 g

m.p.: 91-92.5° C.

### Step 6: 3,4-Dimethyl-8-methoxy-1-propyl-imidazo[1,5-a]-pyrido[3,2-e]-pyrazine Hydrochloride (Example 39a)

To a solution of 13.52 g of pure 3,4-dimethyl-8-methoxy-1-propyl-imidazo-[1,5-a]-pyrido[3,2-e]-pyrazine and 100 ml of dichloromethane 2.5 equivalents of hydrochloric acid dissolved in 100 ml of water were added. The mixture was vigorously stirred. The dichloromethane layer was then separated and subsequently the water layer was extracted for 6 times with 100 ml dichloromethane. After evaporation of the dichloromethane yellowish crystals remains. (yield 85%; yellowish crystals; m. p. 171-175° C.).

Yield: 13.05 g

m.p.: 171-175° C.

Surprisingly, the compounds of formula (IIa) are potent inhibitors of the enzyme PDE10. A substance is considered to effectively inhibit PDE10 if it has an IC₅₀ of less than 10 μM, preferably less than 1 μM.

### Example 1b

### 4-amino-8-methoxy-3-methyl-1-propyl-imidazo[1,5-a]pyrido[3,2-e]pyrazine

10 g of intermediate A1 and 200 ml of an aqueous solution of NH₃ (32%) are mixed in an autoclave and heated up to 130° C. for 8 hours. The reaction mixture is diluted with 200 ml water. The precipitated reaction product is separated washed with water and dichloro methane and dried at reduced pressure.

Yield: 8.5 g

m.p.: 219-221° C.

The following examples are prepared using the same route of synthesis and reaction conditions like described above for example 1b:

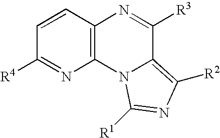

| Example | R¹ | R² | R³ | R⁴ | Fp [° C.] |
|---|---|---|---|---|---|
| 1b | —C₃H₇ | —CH₃ | —NH₂ | —OCH₃ | 219-221 |
| 2b | —C₂H₅ | —CH₃ | —NH₂ | —OCH₃ | 215-217 |
| 3b | —C₂H₅ | —CH₃ | —NH₂ | —H | 190-191 |
| 4b | —C₃H₇ | —CH₃ | —NH₂ | —H | 163-165 |
| 5b | —C₂H₅ | —CH₃ | —NH₂ | 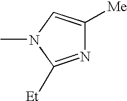 | 277-281 |
| 6b | —C₃H₇ | —CH₃ | —NH₂ | 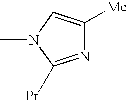 | 215-221 |
| 7b | —C₆H₁₃ | —CH₃ | —NH₂ | —OCH₃ | 167-169 |
| 8b | —CH₂CH₂CF₃ | —CH₃ | —NH₂ | —OCH₃ | 273-276 |
| 9b | —(CH₂)₂C₆H₅ | —CH₃ | —NH₂ | —OCH₃ | 198-200 |
| 10b | —C₆H₅ | —CH₃ | —NH₂ | —OCH₃ | 248-250 |
| 11b | —C₆H₄(2-Cl) | —CH₃ | —NH₂ | —OCH₃ | 248-250 |
| 12b | —C₆H₄(4-F) | —CH₃ | —NH₂ | —OCH₃ | 245-251 |
| 13b | —CH(CH₃)₂ | —H | —NH₂ | —OCH₃ | 277-279 |
| 14b | —H | —H | —NH₂ | —OCH₃ | 239-241 |
| 15b | —H | —C₆H₅ | —NH₂ | —OCH₃ | 252-253 |
| 16b | —C₃H₇ | —CH₃ | —NHCH₃ | —OCH₃ | 111-113 |
| 17b | —C₃H₇ | —CH₃ | —NHC₂H₅ | —OCH₃ | 140-142 |
| 18b | —C₂H₅ | —CH₃ | —NHCH₃ | —OCH₃ | 172-174 |
| 19b | —C₃H₇ | —CH₃ | —N(CH₃)₂ | —OCH₃ | 93-95 |
| 20b | —C₂H₅ | —CH₃ | —NHC₄H₉ | —OCH₃ | 62-65 |
| 21b | —C₂H₅ | —CH₃ | —NHCH₂C₆H₅ | —OCH₃ | 127-128 |
| 22b | —C₂H₅ | —CH₃ | 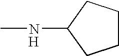 | —OCH₃ | 93-95 |
| 23b | —C₃H₇ | —CH₃ | 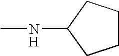 | —OCH₃ | 77-80 |
| 24b | —C₂H₅ | —CH₃ | 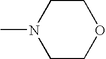 | —OCH₃ | 132-133 |
| 25b | —CH₂CH₂CF₃ | —CH₃ | 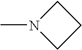 | —OCH₃ | 150-152 |
| 26b | —C₃H₇ | —CH₃ | 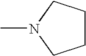 | —OCH₃ | 71-74 |
| 27b | —C₃H₇ | —CH₃ | 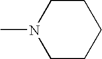 | —OCH₃ | 127-129 |
| 28b | —C₂H₅ | —CH₃ | 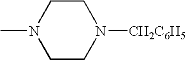 | —OCH₃ | 224-227 |
| 29b | —C₃H₇ | —CH₃ | 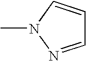 | —OCH₃ | 125-126 |
| 30b | —C₃H₇ | —CH₃ | 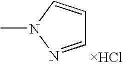 | —OCH₃ | 185-188 |
| 31b | —C₃H₇ | —CH₃ | 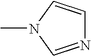 | —OCH₃ | 137-139 |
| 32b | —C₃H₇ | —CH₃ | 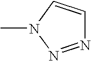 | —OCH₃ | 218-220 |
| 33b | —C₃H₇ | —CH₃ | 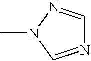 | —OCH₃ | 167-169 |
| 34b | —C₃H₇ | —CH₃ | 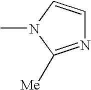 | —OCH₃ | 163-164 |
| 35b | —C₃H₇ | —CH₃ | 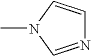 | —OH | 313-320 |

### Example 36b

### 1-ethyl-4-(N-formyl-amino)-8-methoxy-3-methyl-imidazo[1,5-a]pyrido[3,2-e]pyrazine

A mixture of 2.1 ml of methane carboxylic acid and 5 ml of acetic acid anhydride is stirred at 60-70° C. for 1 hour. At room temperature 1 g of 4-amino-1-ethyl-8-methoxy-3-methyl-imidazo[1,5-a]pyrido[3,2-e]pyrazine (example 2) is added. After stirring for 5 hours at 30° C. the mixture is neutralized by addition of NaHCO₃ solution. The crude product is collected washed with water and dried at 40° C. For the final purification column chromatography is used (dichloro methane/methanol 3:1).

Yield: 0.6 g

m.p.: 206-208° C.

The following examples are prepared using the same route of synthesis and reaction conditions as described above for Example 26b:

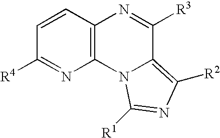

| Ex- | | | | | |
|---|---|---|---|---|---|
| ample | R¹ | R² | R³ | R⁴ | Fp [° C.] |
| 36b | —C₂H₅ | —CH₃ | —NHCH═O | —OCH₃ | 206-208 |
| 37b | —C₃H₇ | —CH₃ | —NHCH═O | —OCH₃ | 205-207 |
| 38b | —C₃H₇ | —CH₃ | —NH(C═O)CH₃ | —OCH₃ | 210-213 |
| 39b | —C₃H₇ | —CH₃ | —N[(C═O)CH₃]₂ | —OCH₃ | 135-138 |
| 40b | —C₂H₅ | —CH₃ | —NH(C═O)CH₃ | —OCH₃ | 199-202 |
| 41b | —C₂H₅ | —CH₃ | —N[(C═O)CH₃]₂ | —OCH₃ | 135-136 |
| 42b | —C₆H₅ | —CH₃ | —NH(C═O)CH₃ | —OCH₃ | 190-192 |
| 43b | —C₃H₇ | —CH₃ | —NH(C═O)C₂H₅ | —OCH₃ | 193-195 |
| 44b | —C₃H₇ | —CH₃ | 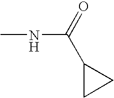 | —OCH₃ | 200-201 |

Surprisingly, the compounds of formula (IIb) and (IIc) are potent inhibitors of the enzyme PDE10. A substance is considered to effectively inhibit PDE10 if it has an IC₅₀ of less than 10 μM, preferably less than 1 μM.

### Preparation and Characterization of PDE10

### Method A

Phosphodiesterase isoenzyme 10 (PDE10) activity was determined in preparations of human recombinant PDE10A and PDE10 from pig striatum, respectively.

The DNA of PDE10A1 (AB 020593, 2340 bp) was synthesized and cloned into the vector pCR4.TOPO (Entelechon GmbH, Regensburg, Germany). The gene was than inserted into a baculovirus vector, ligated with the baculovirus DNA. The enzyme-protein was expressed in SF21-cells. The enzyme was isolated from these cells by harvesting the cells by a centrifugation at 200 g to collect the cells. The cells were resuspended in 50 mM Tris-HCl/5 mM MgCl₂ buffer (pH=7.4) and lysed by a sonication of the cells. The cytosolic PDE10A was obtained by a centrifugation at 48000 g for 1 h in the supernatant and stored at −70° C.

Striatum from male hybrid pigs (150 kg) were collected and frozen at −70° C.

At the day of preparation 0.5 g striatum was homogenised in 10 ml 50 mM Tris/Mg-buffer at 4° C. and centrifuged for one hour at 100000 g. The supernatant was removed and the pellet was resuspended in the same buffer, but containing 1% Triton and incubated for 45 min at 4° C. The membrane fraction was applied onto a 5 ml Hi Trap™ QHP column at the Akta-FPLC. After washing the column the bound PDE protein was eluted with an increasing sodium chloride gradient (0 mM-500 mM sodium chloride) in 50 mM Tris/Mg-buffer at 4° C. in the presence of 1% Triton. The eluted and collected fractions were tested with 100 nM [3H]-cAMP for PDE10-activity in the presence and without a specific PDE-Inhibitor at a concentration, were a 100% inhibition is expected. The fractions with PDE10-activity were pooled and frozen in aliquots until use at −20° C.

PDE10 activity was determined in a one step procedure in microtiter plates. The reaction mixture of 100 μl contained 50 mM Tris-HCl/5 mM MgCl₂ buffer (pH=7.4) (Sigma, Deisenhofen, Germany; Merck, Darmstadt, Germany) 0.1 μM [3H]-cAMP (Amersham, Buckinghamshire, UK) and the enzyme. Nonspecific activity was tested without the enzyme. The reaction was initiated by addition of the substrate solution and was carried out at 37° C. for 30 minutes. Enzymatic activity was stopped by addition of 25 μl YSi-SPA-beads (Amersham-Pharmacia). One hour later the mixture was measured in a liquid scintillation counter for microtiterplates (Microbeta Trilux). To pipette the incubation mixture a robot Biomek (Fa. Beckman) is used. The determined Km-values for the substrate cAMP is 88 nM for pig striatum and 130 nM for human recombinant PDE10A respectively. The optimal amount of enzyme in the assay has been determined and optimised for each enzyme preparation before using the enzyme in compound testing. For determination of IC50 values the Hill-plot, 2-parameter-model, was used. Specific inhibitors of other PDE-Subtypes do not inhibit the PDE10 preparation significantly. Papaverine was used as the most common PDE10 inhibitor and inhibits the PDE10 with IC50 values of 89 nM and 103 nM for PDE10 from human recombinant PDE10A and PDE10 from striatum of pig respectively.

### Method B

Phosphodiesterase isoenzyme 10 (PDE10) activity was determined in preparations of rat, pig and guinea pig striatum respectively. Striatum from male Wistar rats (180-200 g), male hybrid pigs (150 kg) and male guinea pigs (CRL (HA), 500 g) respectively were collected and frozen at −70° C.

In the prepared brain areas gene segments containing the catalytic domain of the PDE10 were amplified and the sequence determined. Therefore the RNA from the frozen striatum of the different animals was isolated according to the instructions of the RNeasy kit (Qiagen; Hilden; Germany) and transcribed into cDNA using Oligo-Primer provided with the 1^{st} strand cDNA synthese kit for RT-PCR (Roche; Mannheim; Germany). These cDNA was used as template for the PCR-reaction to amplify the catalytic domain of the PDE10. For the PCR reaction Taq-Polymerase (Promega; Mannheim; Germany) was used. Therefore it was possible to clone the amplificates directly by TA-cloning in the pCR2.1 vector (Invitrogen; Karlsruhe; Germany). The cloning vector was transformed into E. coli's (XL-2), replicated within the cells, prepared and the included gene sequence determined for the pig and the guinea pig.

The following primers were used for the PCR-reaction:

| | | |
|---|---|---|
| P1: | tgcatctacagggttaccatggagaa | (SEQ ID NO:1) |
| P2: | tatccctgcaggccttcagcagaggctct | (SEQ ID NO:2) |
| P3: | ttcacatggatatgcgacggtaccttct | (SEQ ID NO:3) |
| P4: | ctgtgaagaagaactatcggcgggttcctta. | (SEQ ID NO:4) |

For the pig the priming was successful with P1 and P2. The following sequence (SEQ ID NO: 5) was identified:

tgcatctacagggttaccatggagaagctgtcctaccacagcatttgtac
cgcggaagagtggcaaggcctcatgcgcttcaaccttcccgtccgtcttt
gcaaggagattgaattgttccacttcgacattggtccttttgaaaacatg
tggcctggaatctttgtctatatggttcatcgcttctgtgggacggcctg
ctttgagcttgaaaagctgtgtcgttttatcatgtctgtgaagaagaact
atcgtcgggttccttaccacaactggaagcacgcggtcacggtggcacac
tgcatgtacgccatcctccagaacagccacgggctcttcaccgacctcga
gcgcaaaggactgctaatcgcgtgtctgtgccacgacctggaccacaggg
gcttcagcaacagctacctgcagaaattcgaccaccccctggccgctctc
tactccacgcccaccatggagcagcaccacttctcccagaccgtgtccat
cctccagttggaagggcacaacatcttctccaccctgagctccagtgagt
acgagcaggtgcttgagatcatccgcaaagccatcattgccacagacctc
gctttgtactttggaaacaggaaacagttggaggagatgtaccagaccgg
atcgctaaaccttaataaccagtcacatagagaccgcgtcattggtttga
tgatgactgcctgtgatctctgttccgtgacaaaactgtggccagtaaca
aaactgacggcaaatgatatatatgcggaattctgggccgagggcgatga
ggtgaagaagctgggaatacagcctattcccatgatggacagagacaaga
aggacgaagtcccacaaggccagctcggattctacaacgcggtagctatc
ccctgctacaccaccctcacccagatcttcccgcccacagagcctcttct
gaaggcctgcagggata

For the guinea pig the priming was successful with P4 and P2 as well as for P2 and P3.

The following sequence (SEQ ID NO:6) was identified with P4 and P2:

ctgtgaagaagaactatcggcgggttccttaccacaactggaagcatgca
gtcacggtggcgcactgcatgtacgccatacttcaaaacaacaatggcct
cttcacagaccttgagcgcaaaggcctgctaattgcctgtctgtgccatg
acctggaccacaggggcttcagtaacagctacctgcagaaattcgaccac
cccctggctgcgttgtactccacctccaccatggagcaacaccacttctc
ccagacggtgttcatcctccagctggaaggacacaacatcttctccaccc
tgagctccagcgagtacgagcaggtgctggagatcatccgcaaagccatc
atcgccactgacctcgcactgtactttgggaacaggaagcagttggagga
gatgtaccagacagggtcgctgaacctcaataaccagtcccatcgagacc
gcgtcatcggcttgatgatgactgcctgcgatctttgctctgtgacgaaa
ctatggccagttacaaaattgacagcaaatgatatatatgcagagttctg
ggctgagggggatgagatgaagaagttggggatacagcccatccctatga
tggacagagacaagaaggatgaagtccctcaaggacagcttggattctac
aatgctgtggccatcccctgctataccaccctgacgcagatcctcccacc
cacagagcctctgctgaaggcctgcagggata

The following sequence (SEQ ID NO:7) was identified with P2 and P3:

tagagcctctgctgaaggcctgcagggataacctcaatcagtgggagaag
gtaattcgaggggaagagacagcaatgtggatttcaggcccagcaactag
caaaagcacatcagggaagccgaccaggaaggtcgatgactgatcctgag
gtgatgtctgcctagcaactgactcaacctgcttctgtgacttcgttctt
tttatttttatttttttaacggggtgaaaacctctctcagaaggtaccgt
cgcatatccatgtgaa

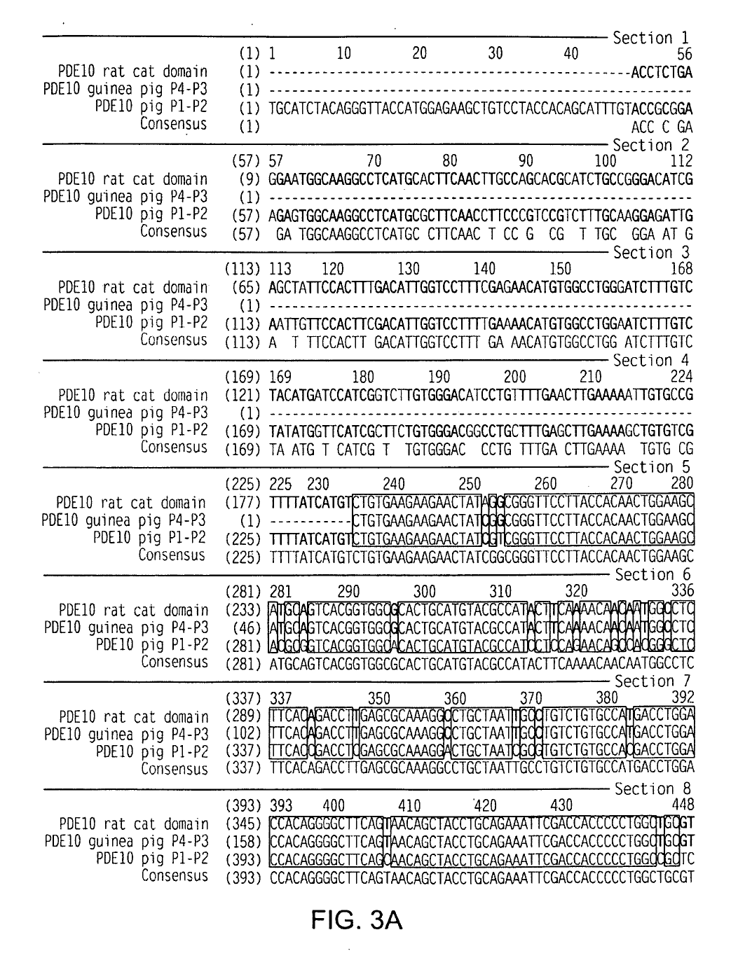

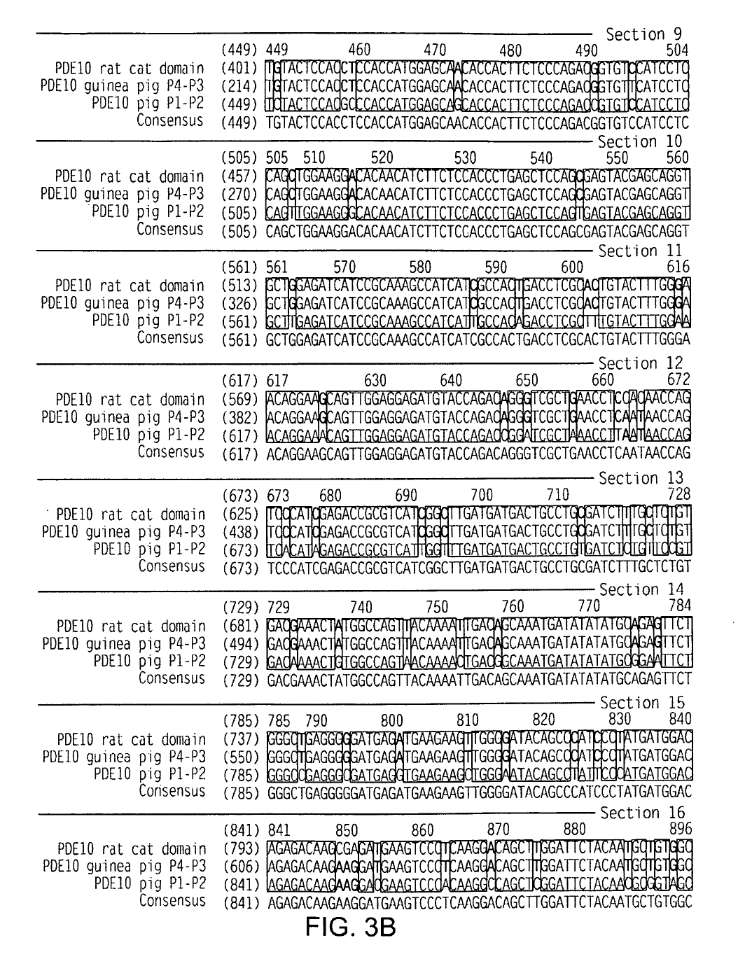

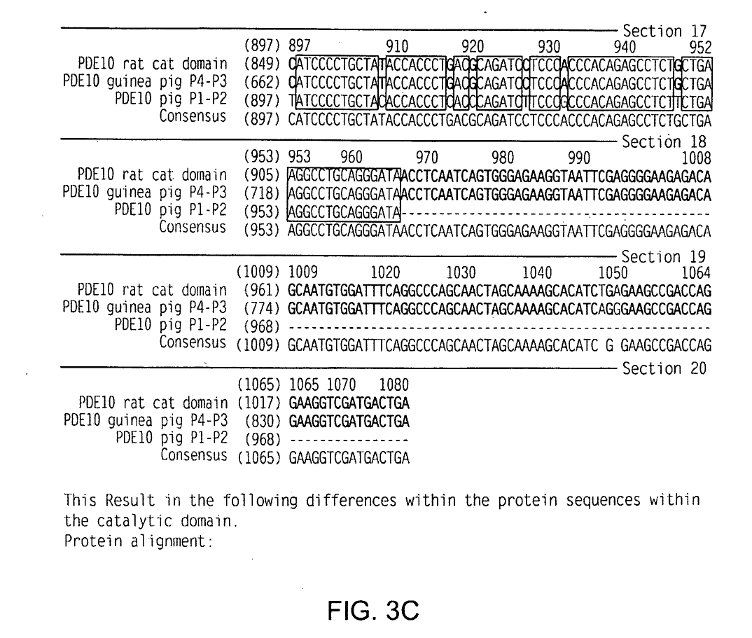

An alignment of the sequences showed a nearly complete accordance between the rat (published gene number NM_{—}022236 3437 bp; coding sequence: 281-2665; catalytic domain 1634-2665) and the guinea pig. More differences were detect between rat and pig. For the alignment the coding areas are used only. The gene alignment is shown in FIG. 3.

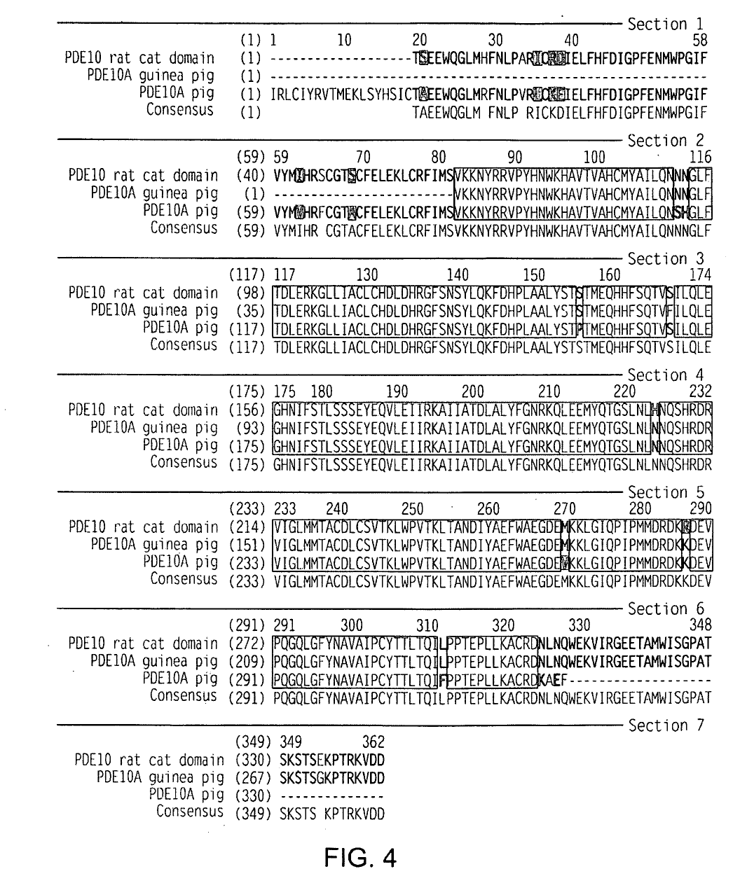

This results in the following differences in the protein sequences within the catalytic domain as shown in a protein alignment (FIG. 4).

For the enzymatic testing of PDE10 activity 0.5 g of the isolated and frozen striatum was homogenised in 10 ml 50 mM Tris/Mg-buffer at 4° C. and centrifuged for one hour at 100000 g. The supernatant is called the cytosolic fraction and was removed and stored on ice. The pellet was resuspended in the same buffer, but containing 1% Triton and incubated for 45 min at 4° C. Both fractions were independently applied onto a 5 ml Hi Trap™ QHP column at the Äkta-FPLC. After washing the columns the bound PDE protein was eluted with an increasing sodium chloride gradient (0 mM-500 mM sodium chloride) in 50 mM Tris/Mg-buffer at 4° C. for the cytosolic fraction and in the presence of 1% Triton for the membrane fraction. The eluted and collected fractions were tested with 100 nM [³H]-cAMP for PDE10-activity in the presence and without a specific PDE-Inhibitor at a concentration, were a 100% inhibition is expected. The fractions with PDE10-activity were pooled and frozen in aliquots until use at −20° C.

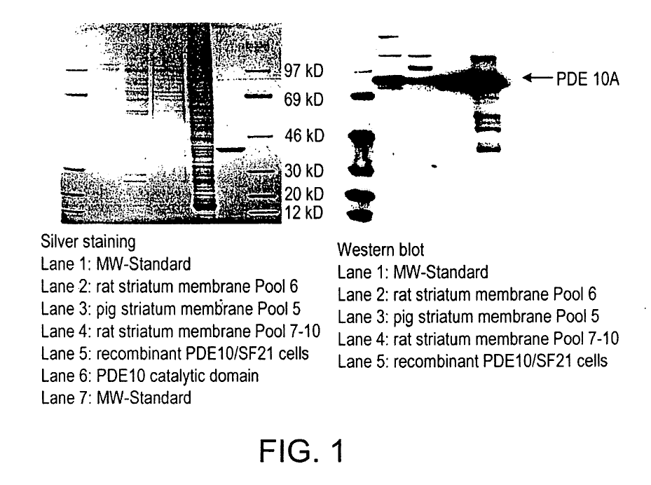

The pooled fractions from the FPLC were additional characterized by Western blot. It was shown, that the PDE10A containing pooled fractions include a great number of other cellular proteins. Nevertheless PDE10 was detected with specific antibodies by Western blot clearly (FIG. 1).

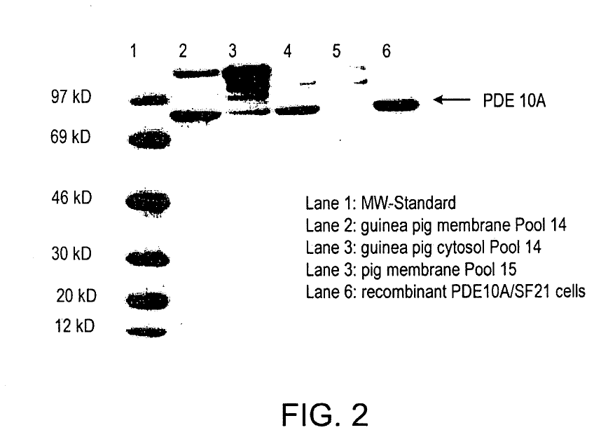

The protein was proven in the preparation of the striatum of the rat, the pig and the guinea pig. The main part of protein was found in the membrane fraction (FIG. 2).

### Inhibition of PDE10

PDE10 activity was determined in a one step procedure in microtiter plates. The reaction mixture of 100 μl contained 50 mM Tris-HCl/5 mM MgCl₂ buffer (pH=7.4) (Sigma, Deisenhofen, Germany; Merck, Darmstadt, Germany) 0.1 μM [³H]-cAMP (Amersham, Buckinghamshire, UK) and the enzyme. Nonspecific activity was tested without the enzyme. The reaction was initiated by addition of the substrate solution and was carried out at 37° C. for 30 minutes. Enzymatic activity was stopped by addition of 25 μl YSi-SPA-beads (Amersham-Pharmacia). One hour later the mixture was measured in a liquid scintillation counter for microtiterplates (Microbeta Trilux). To pipette the incubation mixture a robot Biomek (Fa. Beckman) is used. The determined Km-values for the substrate cAMP is 78 nM for PDE10 from rat striatum, 88 nM for pig striatum and 66.7 nM for guinea pig striatum respectively. cGMP is the second substrate for PDE10, the Km values are 1800 nM, 2200 nM and 1700 nM for PDE10 from these species. For the test with cGMP 500 nM of this substrate was used. The optimal amount of enzyme in the assay has been determined and optimised for each enzyme preparation and substrate separately before using the enzyme in compound testing. For determination of IC₅₀ values the Hill-plot, 2-parameter-model, was used. Specific inhibitors of other PDE-Subtypes do not inhibit the PDE10 preparation significantly. Papaverine was used as the most common PDE10 inhibitor and inhibits the PDE10 with IC₅₀ values of 142 nM, 110 nM and 77 nM for PDE10 from striatum of rat, pig and guinea pig respectively.

| | Inhibition of PDE10 from rat |
|---|---|
| Example | IC₅₀ [μM] |
| 35a | 0.061 |
| 38a | 0.012 |
| 62a | 0.035 |
| 63a | 0.563 |
| 69a | 0.011 |
| 70a | 0.072 |
| 91a | 0.159 |
| 95a | 0.335 |

| | Inhibition of PDE10 from pig |
|---|---|
| Example | IC₅₀ [μM] |
| 1a | 0.010 |
| 29a | 0.013 |
| 30a | 0.020 |
| 31a | 0.171 |
| 35a | 0.040 |
| 38a | 0.006 |
| 39a | 0.005 |
| 40a | 0.024 |
| 41a | 0.118 |
| 42a | 0.059 |
| 43a | 0.035 |
| 44a | 0.003 |
| 45a | 0.053 |
| 46a | 0.049 |
| 47a | 0.006 |
| 48a | 0.007 |
| 49a | 0.001 |
| 52a | 0.053 |
| 53a | 0.043 |
| 54a | 0.018 |
| 55a | 0.014 |
| 57a | 0.011 |
| 58a | 0.002 |
| 59a | 0.011 |
| 60a | 0.023 |
| 62a | 0.006 |
| 63a | 0.189 |
| 65a | 0.559 |
| 66a | 0.752 |
| 67a | 0.083 |
| 68a | 0.141 |
| 69a | 0.005 |
| 71a | 0.126 |
| 72a | 0.088 |
| 73a | 0.019 |
| 75a | 0.078 |
| 79a | 0.011 |
| 80a | 0.037 |
| 84a | 0.025 |
| 85a | 0.013 |
| 86a | 0.023 |
| 87a | 0.015 |
| 91a | 0.108 |
| 95a | 0.222 |

| | Inhibition of PDE10 from guinea pig |
|---|---|
| Example | IC₅₀ [μM] |
| 29a | 0.018 |
| 30a | 0.051 |
| 38a | 0.019 |
| 47a | 0.015 |
| 58a | 0.004 |
| 62a | 0.026 |
| 69a | 0.011 |

The compounds of formula (IIa) show significant antipsychotic effects on the MK-801-induced hyperactivity and stereotyped sniffing, an animal model of psychosis.

| | Inhibition of PDE10 from rat |
|---|---|
| Example | IC₅₀ [μM] |
| 1b | 0.006 |
| 3b | 0.043 |
| 4b | 0.057 |
| 10b | 0.005 |
| 36b | 0.241 |
| 37b | 0.050 |
| 40b | 0.220 |
| 42b | 0.095 |
| 43b | 2.410 |
| 44b | 2.180 |

| | Inhibition of PDE10 from pig |
|---|---|
| Example | IC₅₀ [μM] |
| 2b | 0.015 |
| 3b | 0.041 |
| 4b | 0.027 |
| 5b | 0.006 |
| 6b | 0.001 |
| 7b | 0.048 |
| 9b | 0.038 |
| 10b | 0.003 |
| 11b | 0.0005 |
| 12b | 0.006 |
| 26b | 0.137 |
| 27b | 0.302 |
| 29b | 0.199 |
| 30b | 0.155 |
| 31b | 0.009 |
| 33b | 0.025 |
| 34b | 0.395 |
| 35b | 0.086 |
| 36b | 0.080 |
| 37b | 0.029 |
| 42b | 0.041 |
| 43b | 0.896 |
| 44b | 0.671 |

| | Inhibition of PDE10 from guinea pig |
|---|---|
| Example | IC₅₀ [μM] |
| 3b | 0.037 |
| 11b | 0.001 |
| 31b | 0.011 |

Surprisingly, also the intermediates A of the synthesis of compounds of formula II are potent inhibitors of the enzyme PDE10.

| | Inhibition of PDE10 from rat |
|---|---|
| Intermediate | IC₅₀ [μM] |
| A1 | 0.008 |
| A2 | 0.023 |
| A11 | 0.171 |
| A14 | 0.237 |

| | Inhibition of PDE10 from pig |
|---|---|
| Intermediate | IC₅₀ [μM] |
| A1 | 0.004 |
| A2 | 0.017 |
| A4 | 0.002 |
| A5 | 0.071 |
| A6 | 0.056 |
| A7 | 0.034 |
| A9 | 0.004 |
| A11 | 0.097 |
| A12 | 0.038 |
| A13 | 0.053 |
| A14 | 0.128 |
| A19 | 0.009 |
| A20 | 0.011 |
| A21 | 0.005 |
| A22 | 0.052 |
| A23 | 0.003 |
| A24 | 0.002 |
| A25 | 0.063 |
| A26 | 0.046 |
| A28 | 0.008 |

### Test Procedure:

Female Wistar rats (Crl: (WI) BR, Charles River, Sulzfeld, Germany) weighing 150 to 180 g were used for the MK-801-induced psychosis. Animals were housed under standard conditions in groups of five on a 12 h light/dark cycle (light on at 0600 h) with ad libitum access to food (Pellets, ssniff M/R 15, Spezialdiät GmbH, Soest/Westfalen) and water.

MK-801 (dizocilpine, MW 337.37) was obtained by Tocris, distributed by Biotrend Chemikalien GmbH, Köln, Germany.

### Drug Administration Schedule/Dosage:

| | dosage | pre-treatment | number of | route of |
|---|---|---|---|---|
| substance | [mg/kg] | [min] | application [n] | administration |
| MK-801 | 0.1 | 10 | 1 | i.p. |
| Example 91a | 15, 30 | 30 | 1 | i.p. |
| Example 35a | 10, 30 | 30 | 1 | p.o. |
| Example 95a | 10, 30 | 30 | 1 | p.o. |
| Example 62a | 2.5, 5.0 | 30 | 1 | p.o. |
| Example 38a | 1.0, 2.5, | 30 | 1 | p.o. |
| | 5.0 | | | |
| Example 69a | 1.0, 2.5, | 30 | 1 | p.o. |
| | 5.0 | | | |
| Example 29a | 2.5, 5.0, | 30 | 1 | p.o. |
| | 7.5, 10 | | | |
| Example 47a | 5.0, 7.5, | 30 | 1 | p.o. |
| | 10 | | | |
| Example 30a | 5.0, 10, | 60 | 1 | p.o. |
| | 15, 20 | | | |
| Example 55a | 10, 30 | 30 | 1 | p.o. |

| | Dosage | pre-treatment | number of | Route of |
|---|---|---|---|---|
| substance | [mg/kg] | [min] | application [n] | administration |
| MK-801 | 0.1 | 10 | 1 | i.p. |
| Example 1b | 10, 30 | 30 | 1 | p.o. |
| Example | 0.5, 1.0, | 30 | 1 | p.o. |
| 11b | 2.5, 5.0, | | | |
| | 10 | | | |

### Preparation of Compounds:

Compounds were freshly suspended in 0.5% hydroxyethylcellulose so that an administration volume of 0.5 ml/100 g was reached for each substance and dose. Hydroxyethylcellulose was solved in distilled water.

MK-801 was solved in saline so that an administration volume of 0.5 ml/100 g was reached. The suspensions and solution were placed on a magnetic stirrer before and during dosing procedures.

The behaviour induced by the NMDA antagonist MK-801 is generally accepted as a rat model of psychosis. MK-801 induces stereotyped sniffing, hyperactivity and ataxia in rats after intraperitoneal administration.

Locomotor activity of the rats was recorded by the MotiTest Apparatus (TSE, Bad Homburg, Germany). The test area consisted of a squared arena (45×45 cm) with protective plexiglass walls (20 cm of height) where rats could freely move. Horizontal movements were recorded by 32 infrared photocells arranged along the bottom of each wall of the arena. The activity [sec] was measured by the computer program “ActiMot” (TSE, Bad Homburg, Germany).

Stereotyped sniffing was scored by the experimenter every five minutes for one hour (12 intervals) according to the method described by Andine et al. (1999). The scores of the 12 intervals were summed up at the end of the recording time.

| score | stereotyped sniffing |
|---|---|
| 0 | no stereotyped sniffing |
| 1 | discontinuous sniffing (free interval > 5 s) |
| 2 | continuous sniffing |

The day of experiment the female rats were placed in the laboratory and received the test compound or vehicle at the appropriate time prior to test. MK-801 0.1 mg/kg was intraperitoneally administered 10 minutes prior to test.

At the beginning of the test the rats were placed in the centre of the squared arena of the MotiTest apparatus. Behaviour of the rats was recorded for one hour. After each run animals were removed and the boxes thoroughly cleaned and dried.

### Statistics:

Results were analysed by one way analysis of variance (ANOVA). Tukey test was used for individual comparison. P<0.05 was regarded as significant.

### Results:

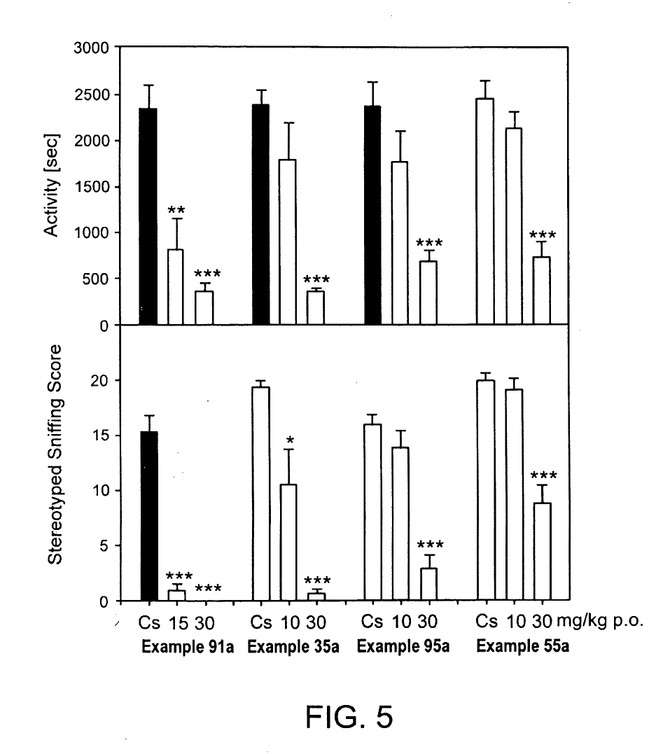

The results are shown in FIGS. 5, 6, 7, 8, and 9.

FIG. 5 shows the effect of the compounds of Example 91a, 35a, 95a and 55a on 25 MK-801-induced psychosis

MK-801 at 0.1 mg/kg i.p. was administered 10 min before testing. Compounds at the described doses were administered 30 min prior to the test. Activity and stereotyped sniffing was recorded for 1 h. Cs=control with MK-801 stimulation. Significant to MK-801 stimulated control (=Cs): * p<0.05, *** p<0.001.

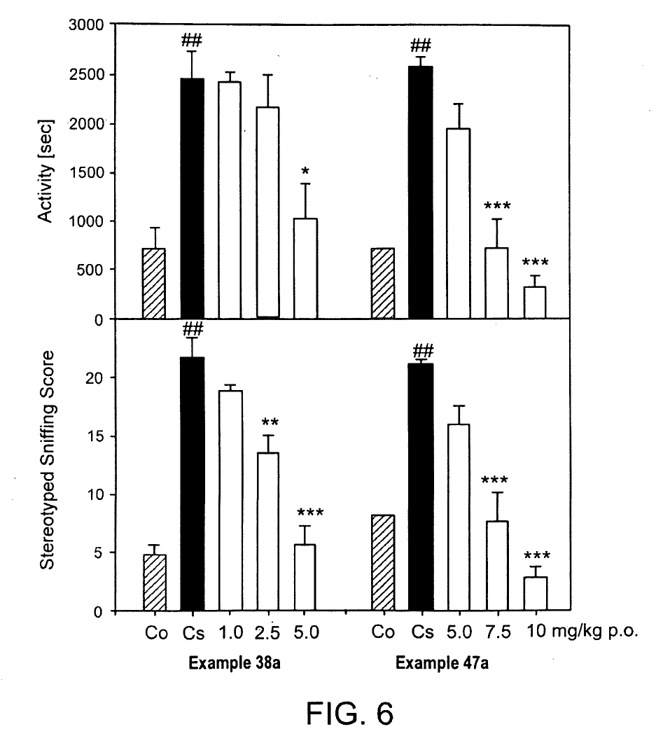

FIG. 6 shows the effect of the compounds of Example 38a and 47a on MK-801-induced psychosis

MK-801 at 0.1 mg/kg i.p. was administered 10 min before testing. Compounds at the described doses were administered 30 min prior to the test. Activity and stereotyped sniffing was recorded for 1 h. Co=control without MK-801 stimulation. Cs=control with MK-801 stimulation. Significant to non-stimulated control (Co): ## p<0.01, ### p<0.001. Significant to MK-801 stimulated control (Cs): * p<0.05, ** p<0.01, *** p<0.001.

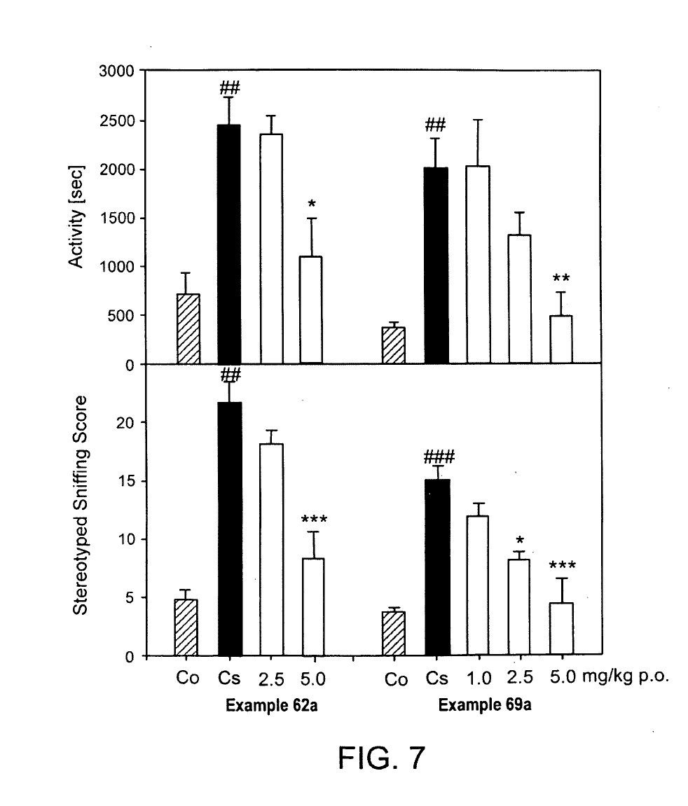

FIG. 7 shows the effect of the compounds of Example 62a and 69a on MK-801-induced psychosis

MK-801 at 0.1 mg/kg i.p. was administered 10 min before testing. Compounds at the described doses were administered 30 min prior to the test. Activity and stereotyped sniffing was recorded for 1 h. Co=control without MK-801 stimulation. Cs=control with MK-801 stimulation. Significant to non-stimulated control (Co): ## p<0.01, ### p<0.001. Significant to MK-801 stimulated control (Cs): * p<0.05, ** p<0.01, *** p<0.001.

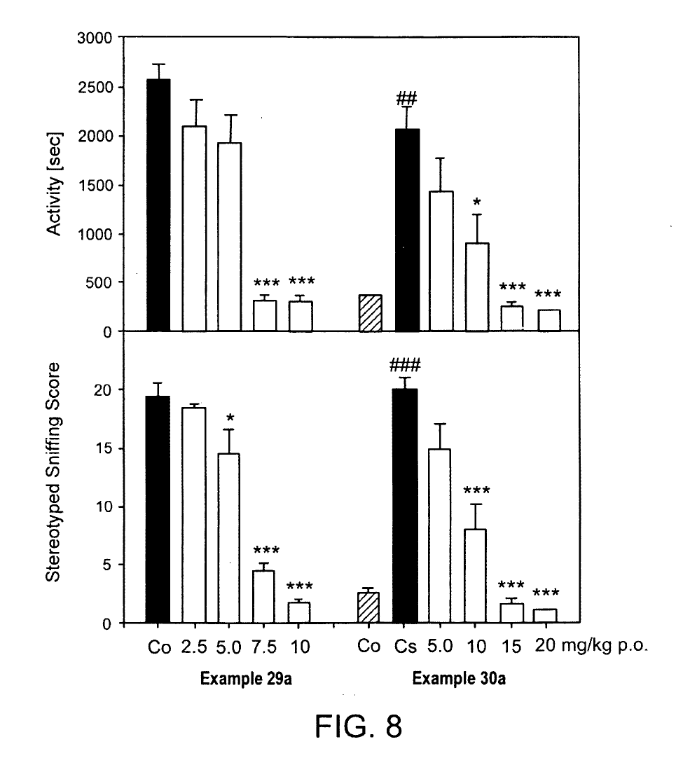

FIG. 8 shows the effect of the compounds of Example 29a and 30a on MK-801-induced psychosis

MK-801 at 0.1 mg/kg i.p. was administered 10 min before testing. Compounds at the described doses were administered 30 min prior to the test. Activity and stereotyped sniffing was recorded for 1 h. Co=control without MK-801 stimulation. Cs=control with MK-801 stimulation. Significant to non-stimulated control (Co): ## p<0.01, ###p<0.001. Significant to MK-801 stimulated control (Cs): * p<0.05, *** p<0.001.

The compound of Example 91a significantly reduced MK-801-induced hyperactivity and stereotyped sniffing starting at 15 mg/kg i.p. The compounds of Example 95a and 55a significantly reversed MK-801-induced hyperactivity and stereotyped sniffing at 30 mg/kg p.o. Example 35a significantly reversed MK-801-induced hyperactivity at 30 mg/kg and stereotyped sniffing starting at 30 mg/kg p.o. The compound of Example 30a significantly reversed MK-801-induced hyperactivity and stereotyped sniffing starting at 10 mg/kg p.o. The compound of Example 47a significantly reversed MK-801-induced hyperactivity and stereotyped sniffing starting at 7.5 mg/kg p.o. Example 29a significantly reversed MK-801-induced hyperactivity starting at 7.5 mg/kg and stereotyped sniffing starting at 5 mg/kg p.o. The compound of Example 62a significantly reversed MK-801-induced hyperactivity and stereotyped sniffing at 5 mg/kg p.o. The compounds of Example 38a and 69a significantly reversed MK-801-induced hyperactivity starting at 5.0 mg/kg and stereotyped sniffing starting at 2.5 mg/kg p.o. The results give evidence for the antipsychotic potential of the compounds.

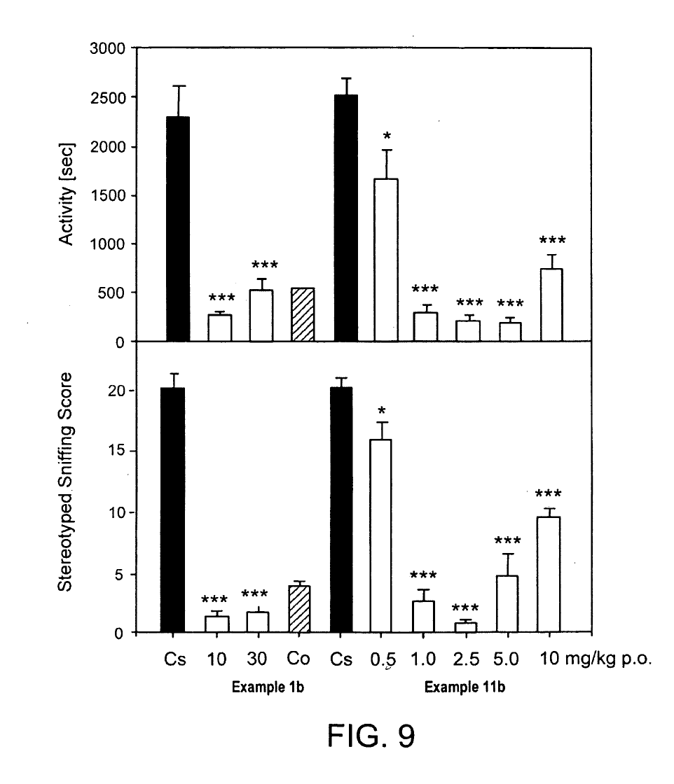

The results are shown in FIG. 9. MK-801 at 0.1 mg/kg i.p. was administered 10 min before testing. The compounds of Example 1b and 11b were administered 30 min prior to the test at the described doses. Activity and stereotyped sniffing was recorded for 1 h. Co=control without MK-801 stimulation, Cs=control with MK-801 stimulation. Significant to MK-801 stimulated control (=Cs): * p<0.05, *** p<0.001.

The compound of Example 1b significantly reversed MK-801-induced hyperactivity and stereotyped sniffing starting at 10 mg/kg p.o. The compound of Example 11b significantly reversed MK-801-induced hyperactivity and stereotyped sniffing starting at 0.5 mg/kg p.o. The results give evidence for the antipsychotic potential of the compounds.

Various modifications of the invention, in addition to those described herein, will be apparent to those skilled in the art from the foregoing description. Such modifications are also intended to fall within the scope of the appended claims. Each reference, including all patents, patent applications, and journal literature, cited in the present application is incorporated herein by reference in its entirety.

What is claimed is:

1. A method of treating or preventing obesity, type 2 diabetes, metabolic syndrome, or glucose intolerance comprising administering to a patient in need a therapeutically effective amount of a compound of Formula (IIa): 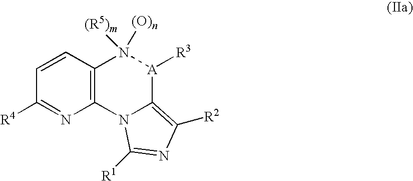
wherein the bond between A and N is a single bond or a double bond,
A is C when the bond is a double bond and CH when the bond is a single bond,
m is 0 or 1,
n is 0 or 1,
wherein R¹ and R² are independently selected from
H,
a cyclic radical,
C₁₋₈ alkyl, optionally mono- or polysubstituted with halo, OH, O—C₁₋₃ alkyl and/or a cyclic radical,
C₂₋₈ alkenyl, optionally mono- or polysubstituted with halo, OH, O—C₁₋₃ alkyl and/or a cyclic radical,
C₂₋₈ alkynyl, optionally mono- or polysubstituted with halo, OH, O—C₁₋₃-alkyl and/or a cyclic radical,
a saturated, monounsaturated or polyunsaturated carboxylic ring system with 3 to 8 atoms, e.g. phenyl, or a heterocyclic ring system with 5 to 15 ring atoms containing at least one heteroatom selected from N including N-oxide, O and S, each optionally mono- or polysubstituted with halo, amino, C₁₋₃ alkylamino, di-C₁₋₃ alkylamino, nitro, C₁₋₃ alkyl, O—C₁₋₃ alkyl, and/or a cyclic radical, and
R³ is selected from
H,
a cyclic radical,
N₃,
CN,
R⁶, OR⁶, SR⁶, SOR⁶, SO₂R⁶,
NH(CO)OR⁶, N((CO)OR⁶)₂, NR⁶((CO)OR⁶),
NH—(C═O)—NH₂, NR⁶—(C═O)—NH₂,
NH—(C═O)—NHR⁶, NR⁶—(C═O)—NHR⁶,
NH—SO₂R⁶, N(SO₂R⁶)₂, and NR⁶(SO₂R⁶),
wherein R⁶ is in each case independently,
a cyclic radical,
C₁₋₈ alkyl, C₃₋₈ cyclo(hetero)alkyl,
C₂₋₈ alkenyl, C₃₋₈ cyclo(hetero)alkenyl,
or C₂₋₈ alkynyl each optionally mono or polysubstituted with halo, OH and/or O—C₁₋₃ alkyl, and/or a cyclic radical,
R⁷, OR⁷, SR⁷, NHSO₂R⁷, N(SO₂R⁷)₂, or N(R⁸)SO₂R⁷,
wherein R⁷ is aryl, heteroaryl, aryl-C₁₋₁₅ alkyl, heteroaryl-C₁₋₅ alkyl,
wherein aryl is phenyl or naphthyl, heteroaryl is an aromatic heterocyclic ring system of 5 to 15 ring atoms containing at least one atom selected from N including N-oxide, S, and O and wherein aryl and heteroaryl are optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical,
R⁸ is C₁₋₅ alkyl, optionally mono or polysubstituted with halo, OH, O—C₁₋₃ alkyl and/or a cyclic radical,
R⁴ is selected from
H,
halo,
a cyclic radical,
R⁹
OH or OR⁹,
NH(C═O)—C₁₋₃ alkyl, optionally mono- or polysubstituted with halo, OH, O—C₁₋₃ alkyl and/or a cyclic radical or
—NH₂, NHR⁹ or NR⁹R¹⁰,
wherein R⁹ and R¹⁰ are independently selected from
a cyclic radical,
C₁₋₆ alkyl or C₃₋₆ cyclo(hetero)alkyl, optionally mono- or polysubstituted with halo, OH, O—C₁₋₃ alkyl and/or a cyclic radical,
aryl-C₁₋₅-alkyl wherein aryl is phenyl, optionally mono- or polysubstituted with halo, amino, C₁₋₃ alkylamino, di-C₁₋₃ alkylamino, nitro, C₁₋₃ alkyl, OH, O—C₁₋₃ alkyl and/or a cyclic radical, or
NR⁹R¹⁰ together form a saturated or unsaturated five-, six- or seven-membered ring which can contain up to 3 heteroatoms, preferably N including N-oxide, S and/or O, optionally mono- or polysubstituted with halo, amino, C₁₋₃ alkylamino, di-C₁₋₃ alkylamino, C₁₋₃ alkyl, O—C₁₋₃ alkyl and/or aryl-C₁₋₅-alkyl, wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical,
and R⁵ is selected from
H,
C₁₋₅ alkyl, C₃₋₆ cycloalkyl or (CO)—C₁₋₅ alkyl, optionally mono or polysubstituted with halo, OH, O—C₁₋₃ alkyl and/or a cyclic radical,
or pharmaceutically acceptable salts thereof.

2. The method of claim 1 wherein said patient is overweight or obese.

3. The method of claim 1 wherein the compound is a selective PDE10 inhibitor.

4. The method of claim 1 further comprising administering a further therapeutic agent.

5. The method of claim 4 wherein said further therapeutic agent is an anti-obesity agent.

6. The method of claim 1 wherein the bond between A and N is a double bond.

7. The method of claim 1 wherein m and n are both 0.

8. The method of claim 1 wherein R¹ is selected from
H,
C₁₋₄ alkyl, particularly C₂₋₄ alkyl optionally mono- or polysubstituted with halo, OH, O—C₁₋₃ alkyl and/or a cyclic radical or
phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical.

9. The method of claim 1 wherein R¹ is C₂₋₄-alkyl.

10. The method of claim 1 wherein R¹ is selected from n-propyl, i-propyl, or phenyl, each optionally substituted.

11. The method of claim 1 wherein R² is
H or
C₁₋₄ alkyl, particularly methyl, optionally substituted, e.g. halo substituted.

12. The method of claim 1 wherein R² is hydrogen, a methyl group, or a trifluoromethyl group.

13. The method of claim 1 wherein R³ is H, CN or C₁₋₃ alkyl.

14. The method of claim 1 wherein R³ is methyl.

15. The method of claim 1 wherein R³ is NH—(C═O)OR⁶

16. The method of claim 1 wherein R³ is NH—(C═O)—OC₁₋₅ alkyl, optionally mono- or polysubstituted.

17. The method of claim 1 wherein R³ is NH—SO₂R⁶.

18. The method of claim 1 wherein R³ is NH—SO₂—C₁₋₅ alkyl, optionally mono- or polysubstituted.

19. The method of claim 1 wherein R⁴ is selected from
H, C₁₋₃ alkyl, O—C₁₋₃ alkyl, NH₂, NHC₁₋₃ alkyl, wherein alkyl is optionally mono- or polysubstituted with halo, OH, O—C₁₋₃ alkyl and/or a cyclic radical or
NH(C═O)—C₁₋₃ alkyl, optionally mono- or polysubstituted with halo, OH, O—C₁₋₃ alkyl and/or a cyclic radical or
cyclopropyl, cyclobutyl, tetrahydropyrrolyl, pyrrolyl, pyrazolyl, imidazolyl, 1,2,3-triazolyl, 1,2,4-triazolyl, piperidinyl, morpholinyl, piperazinyl, optionally mono- or polysubstituted with halo, OH, C₁₋₅ alkyl and/or O—C₁₋₃ alkyl, or aryl-C₁₋₅-alkyl, wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical.

20. The method of claim 1 wherein R⁴ is H, C₁₋₃ alkyl or O—C₁₋₃ alkyl.

21. The method of claim 1 wherein H or OCH₃.

22. The method of claim 1 wherein said compound is selected from:
4,8-dimethoxy-3-methyl-1-propyl-imidazo[1,5-a]pyrido[3,2-e]pyrazine;
4,8-dimethoxy-1-propyl-imidazo[1,5-a]pyrido[3,2-e]pyrazine;
4,8-dimethoxy-1-ethyl-3-methyl-imidazo[1,5-a]pyrido[3,2-e]pyrazine;
4,8-dimethoxy-1,3-dimethyl-imidazo[1,5-a]pyrido[3,2-e]pyrazine;
4,8-dimethoxy-3-methyl-imidazo[1,5-a]pyrido[3,2-e]pyrazine;
1-ethyl-4-isopropyloxy-8-methoxy-3-methyl-imidazo[1,5-a]pyrido[3,2-e]pyrazine;
1-ethyl-8-methoxy-3-methyl-4-propyloxy-imidazo[1,5-a]pyrido[3,2-e]pyrazine;
4-cyclopentyloxy-1-ethyl-8-methoxy-3-methyl-imidazo[1,5-a]pyrido[3,2-e]pyrazine;
4-isopropyloxy-8-methoxy-3-methyl-1-propyl-imidazo[1,5-a]pyrido[3,2-e]pyrazine;
8-methoxy-1,3-dimethyl-4-(2,3,6-trifluorobenzyloxy)-imidazo[1,5-a]pyrido[3,2-e]pyrazine;
4-(2,4-dichlorobenzyloxy)-1-ethyl-8-methoxy-3-methyl-imidazo[1,5-a]pyrido[3,2-e]pyrazine;
4-(2-chloro-6-fluorobenzyloxy)-1-ethyl-8-methoxy-3-methyl-imidazo[1,5-a]pyrido[3,2-e]pyrazine;
1-ethyl-8-methoxy-3-methyl-4-(2,3,6-trifluorobenzyloxy)-imidazo[1,5-a]pyrido[3,2-e]pyrazine;
1-ethyl-8-methoxy-3-methyl-4-(2,4,6-trimethylbenzyloxy)-imidazo[1,5-a]pyrido[3,2-e]pyrazine;
4-(2-chloro-6-fluorobenzyloxy)-8-methoxy-3-methyl-1-propyl-imidazo[1,5-a]pyrido[3,2-e]pyrazine;
4-(2,6-difluorobenzyloxy)-8-methoxy-3-methyl-1-propyl-imidazo[1,5-a]pyrido[3,2-e]pyrazine;
1-ethyl-8-methoxy-3-methyl-4-(2-phenylethyloxy)-imidazo[1,5-a]pyrido[3,2-e]pyrazine;
8-methoxy-3-methyl-4-(2-phenylethyloxy)-1-propyl-imidazo[1,5-a]pyrido[3,2-e]pyrazine;
8-methoxy-1,3-dimethyl-4-(2-phenylethyloxy)-imidazo[1,5-a]pyrido[3,2-e]pyrazine;
8-methoxy-3-methyl-4-(2-phenylethyloxy)-imidazo[1,5-a]pyrido[3,2-e]pyrazine;
8-methoxy-3-methyl-4-(3-phenylpropyloxy)-1-propyl-imidazo[1,5-a]pyrido[3,2-e]pyrazine;
1-ethyl-8-methoxy-3-methyl-4-(3-phenylpropyloxy)-imidazo[1,5-a]pyrido[3,2-e]pyrazine;
1,3-dimethyl-8-methoxy-4-(3-phenylpropyloxy)-imidazo[1,5-a]pyrido[3,2-e]pyrazine;
4-[(3,5-dimethylisoxazol-4-yl)methyloxy]-1-ethyl-8-methoxy-3-methyl-imidazo[1,5-a]pyrido[3,2-e]pyrazine;
1-ethyl-8-methoxy-3-methyl-4-methylthio-imidazo[1,5-a]pyrido[3,2-e]pyrazine;
8-methoxy-3-methyl-4-methylthio-1-propyl-imidazo[1,5-a]pyrido[3,2-e]pyrazine; 1,3-dimethyl-8-methoxy-4-methylthio-imidazo[1,5-a]pyrido[3,2-e]pyrazine;
8-methoxy-3-methyl-4-methylthio-imidazo[1,5-a]pyrido[3,2-e]pyrazine;
4-cyano-8-methoxy-3-methyl-1-propyl-imidazo[1,5-a]pyrido[3,2-e]pyrazine;
4-cyano-8-methoxy-3-methyl-1-ethyl-imidazo[1,5-a]pyrido[3,2-e]pyrazine;
4-azido-8-methoxy-3-methyl-1-propyl-imidazo[1,5-a]pyrido[3,2-e]pyrazine;
8-methoxy-3-methyl-4-methylsulfinyl-1-propyl-imidazo[1,5-a]pyrido[3,2-e]pyrazine;
8-methoxy-3-methyl-4-methylsulfonyl-1-propyl-imidazo[1,5-a]pyrido[3,2-e]pyrazine;
1-ethyl-8-methoxy-3-methyl-4-methylsulfinyl-imidazo[1,5-a]pyrido[3,2-e]pyrazine;
8-methoxy-3-methyl-1-propyl-imidazo[1,5-a]pyrido[3,2-e]pyrazine;
1-ethyl-8-methoxy-3-methyl-imidazo[1,5-a]pyrido[3,2-e]pyrazine;
4-ethyl-8-methoxy-3-methyl-1-propyl-imidazo[1,5-a]pyrido[3,2-e]pyrazine;
3,4-dimethyl-8-methoxy-1-propyl-imidazo[1,5-a]pyrido[3,2-e]pyrazine;
3,4-dimethyl-8-methoxy-1-propyl-imidazo[1,5-a]pyrido[3,2-e]pyrazine hydrochloride;
1-ethyl-3,4-dimethyl-8-methoxy-imidazo[1,5-a]pyrido[3,2-e]pyrazine;
1,3,4-trimethyl-8-methoxy-imidazo[1,5-a]pyrido[3,2-e]pyrazine;
3,4-dimethyl-8-methoxy-1-(3,3,3-trifluoropropyl)-imidazo[1,5-a]pyrido[3,2-e]pyrazine;
3,4-dimethyl-8-methoxy-1-pentyl-imidazo[1,5-a]pyrido[3,2-e]pyrazine;
1-cyclohexyl-3,4-dimethyl-8-methoxy-imidazo[1,5-a]pyrido[3,2-e]pyrazine;
3,4-dimethyl-1-hexyl-8-methoxy-imidazo[1,5-a]pyrido[3,2-e]pyrazine;
3,4-dimethyl-8-methoxy-1-phenethyl-imidazo[1,5-a]pyrido[3,2-e]pyrazine;
3,4-dimethyl-8-methoxy-1-phenyl-imidazo[1,5-a]pyrido[3,2-e]pyrazine;
3,4-dimethyl-8-methoxy-1-phenyl-imidazo[1,5-a]pyrido[3,2-e]pyrazine;
dihydrochloride;
3,4-dimethyl-8-methoxy-1-(2-chlorophenyl)-imidazo[1,5-a]pyrido[3,2-e]pyrazine;
3,4-dimethyl-8-methoxy-1-(4-fluorophenyl)-imidazo[1,5-a]pyrido[3,2-e]pyrazine;
1-propyl-3,4,8-trimethyl-imidazo[1,5-a]pyrido[3,2-e]pyrazine;
1-propyl-3,4-dimethyl-imidazo[1,5-a]pyrido[3,2-e]pyrazine;
1-propyl-4,8-dimethyl-imidazo[1,5-a]pyrido[3,2-e]pyrazine;
8-difluoromethoxy-3,4-dimethyl-1-propyl-imidazo[1,5-a]pyrido[3,2-e]pyrazine;
3,4-dimethyl-8-(piperidin-1-yl)-methoxy-1-propyl-imidazo[1,5-a]pyrido[3,2-e]pyrazine;
3,4-dimethyl-8-(4-methyl-piperazin-1-yl)-methoxy-1-propyl-imidazo[1,5-a]pyrido[3,2-e]pyrazine;
3,4-dimethyl-8-(2-ethyl-4-methyl-imidazol-1-yl)-methoxy-1-propyl-imidazo[1,5-a]pyrido[3,2-e]pyrazine;
3,4-dimethyl-8-(2-propyl-4-methyl-imidazol-1-yl)-methoxy-1-propyl-imidazo[1,5-a]pyrido[3,2-e]pyrazine;
4-difluoromethoxy-3-methyl-1-propyl-imidazo[1,5-a]pyrido[3,2-e]pyrazine-8-ol;
8-methoxy-3-methyl-5-oxo-1-propyl-imidazo[1,5-a]pyrido[3,2-e]pyrazine;
3,4-dimethyl-8-methoxy-5-oxo-1-propyl-imidazo[1,5-a]pyrido[3,2-e]pyrazine;
8-methoxy-4-methoxycarbonylamino-3-methyl-1-propyl-imidazo[1,5-a]pyrido[3,2-e]pyrazine;
4-ethoxycarbonylamino-8-methoxy-3-methyl-1-propyl-imidazo[1,5-a]pyrido[3,2-e]pyrazine;
4-(N,N-bis-methoxycarbonyl)-amino-8-methoxy-3-methyl-1-propyl-imidazo[1,5-a]pyrido[3,2-e]pyrazine;
8-methoxy-4-(methoxycarbonyl-methyl-amino)-3-methyl-1-propyl-imidazo[1,5-a]pyrido[3,2-e]pyrazine;
8-methoxy-3-methyl-4-(3-methyl-ureido)-1-propyl-imidazo[1,5-a]pyrido[3,2-e]pyrazine;
8-methoxy-3-methyl-1-propyl-4-ureido-imidazo[1,5-a]pyrido[3,2-e]pyrazine;
8-methoxy-3-methyl-4-(3-isopropyl-ureido)-1-propyl-imidazo[1,5-a]pyrido[3,2-e]pyrazine;
8-methoxy-3-methyl-4-methylsulfonylamino-1-propyl-imidazo[1,5-a]pyrido[3,2-e]pyrazine;
4-(N,N-bis-methylsulfonyl)-amino-8-methoxy-3-methyl-1-propyl-imidazo[1,5-a]pyrido[3,2-e]pyrazine;
4-ethylsulfonylamino-8-methoxy-3-methyl-1-propyl-imidazo[1,5-a]pyrido[3,2-e]pyrazine;
1-ethyl-8-methoxy-3-methyl-4-methylsulfonylamino-imidazo[1,5-a]pyrido[3,2-e]pyrazine;
8-methoxy-3-methyl-1-propyl-4-trifluoromethylsulfonylamino-imidazo[1,5-a]pyrido[3,2-e]pyrazine;
8-methoxy-3-methyl-1-propyl-4-propylsulfonylamino-imidazo[1,5-a]pyrido[3,2-e]pyrazine;
4-isopropylsulfonylamino-8-methoxy-3-methyl-1-propyl-imidazo[1,5-a]pyrido[3,2-e]pyrazine;
8-methoxy-3-methyl-4-(4-methylphenylsulfonylamino)-1-propyl-imidazo[1,5-a]pyrido[3,2-e]pyrazine;
4-[N,N-bis-(4-methylphenylsulfonyl)-amino]-8-methoxy-3-methyl-1-propyl-imidazo[1,5-a]pyrido[3,2-e]pyrazine;
8-methoxy-3-methyl-1-(3,3,3-trifluoropropyl)-4-methylsulfonylamino-imidazo[1,5-a]pyrido[3,2-e]pyrazine;
1-hexyl-8-methoxy-3-methyl-4-methylsulfonylamino-imidazo[1,5-a]pyrido[3,2-e]pyrazine;
8-methoxy-3-methyl-1-phenethyl-4-methylsulfonylamino-imidazo[1,5-a]pyrido[3,2-e]pyrazine;
8-methoxy-3-methyl-1-phenyl-4-methylsulfonylamino-imidazo[1,5-a]pyrido[3,2-e]pyrazine;
1-(2-chlorophenyl)-8-methoxy-3-methyl-4-methylsulfonylamino-imidazo[1,5-a]pyrido[3,2-e]pyrazine
1-(4-fluorophenyl)-8-methoxy-3-methyl-4-methylsulfonylamino-imidazo[1,5-a]pyrido[3,2-e]pyrazine;
3-methyl-8-(4-methyl-2-propyl-imidazol-1-yl)-1-propyl-4-methylsulfonylamino-imidazo[1,5-a]pyrido[3,2-e]pyrazine;
3-methyl-4-methylsulfonylamino-1-propyl-imidazo[1,5-a]pyrido[3,2-e]pyrazin-8-ol hydrobromide;
3-methyl-4-methylsulfonylamino-1-propyl-imidazo[1,5-a]pyrido[3,2-e]pyrazin-8-ol;
8-difluoromethoxy-3-methyl-4-methylsulfonylamino-1-propyl-imidazo[1,5-a]pyrido[3,2-e]pyrazine;
8-cyclopropylmethoxy-3-methyl-4-methylsulfonylamino-1-propyl-imidazo[1,5-a]pyrido[3,2-e]pyrazine;
3-methyl-1-propyl-4,5-dihydro-imidazo[1,5-a]pyrido[3,2-e]pyrazine;
8-methoxy-1-propyl-4,5-dihydro-imidazo[1,5-a]pyrido[3,2-e]pyrazine;
8-methoxy-3-methyl-1-propyl-4,5-dihydro-imidazo[1,5-a]pyrido[3,2-e]pyrazine;
8-methoxy-3-methyl-1-propyl-4,5-dihydro-imidazo[1,5-a]pyrido[3,2-e]pyrazine hydrochloride;
1-ethyl-8-methoxy-3-methyl-4,5-dihydro-imidazo[1,5-a]pyrido[3,2-e]pyrazine;
3,5-dimethyl-8-methoxy-1-propyl-4,5-dihydro-imidazo[1,5-a]pyrido[3,2-e]pyrazine;
5-acetyl-8-methoxy-3-methyl-1-propyl-4,5-dihydro-imidazo[1,5-a]pyrido[3,2-e]pyrazine and their pharmaceutically acceptable salts.

23. The method of claim 1 wherein said compound is 3,4-Dimethyl-8-methoxy-1-propyl-imidazo[1,5-a]-pyrido[3,2-e]-pyrazine or a pharmaceutically acceptable salt thereof.

24. A method of reducing body fat or body weight in a patient comprising administering to said patient in need a therapeutically effective amount of a compound of formula (IIa): 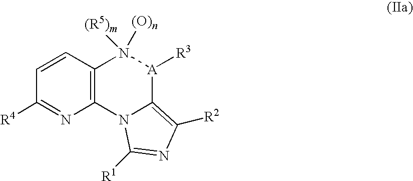
wherein the bond between A and N is a single bond or a double bond,
A is C when the bond is a double bond and CH when the bond is a single bond,
m is 0 or 1,
n is 0 or 1,
wherein R¹ and R² are independently selected from
H,
a cyclic radical,
C₁₋₈ alkyl, optionally mono- or polysubstituted with halo, OH, O—C₁₋₃ alkyl and/or a cyclic radical,
C₂₋₈ alkenyl, optionally mono- or polysubstituted with halo, OH, O—C₁₋₃ alkyl and/or a cyclic radical,
C₂₋₈ alkynyl, optionally mono- or polysubstituted with halo, OH, O—C₁₋₃-alkyl and/or a cyclic radical,
a saturated, monounsaturated or polyunsaturated carboxylic ring system with 3 to 8 atoms, e.g. phenyl, or a heterocyclic ring system with 5 to 15 ring atoms containing at least one heteroatom selected from N including N-oxide, O and S, each optionally mono- or polysubstituted with halo, amino, C₁₋₃ alkylamino, di-C₁₋₃ alkylamino, nitro, C₁₋₃ alkyl, O—C₁₋₃ alkyl, and/or a cyclic radical, and
R³ is selected from
H,
a cyclic radical,
N₃,
CN,
R⁶, OR⁶, SR⁶, SOR⁶, SO₂R⁶,
NH(CO)OR⁶, N((CO)OR⁶)₂, NR⁶((CO)OR⁶),
NH—(C═O)—NH₂, NR⁶—(C═O)—NH₂,
NH—(C═O)—NHR⁶, NR⁶—(C═O)—NHR⁶,
NH—SO₂R⁶, N(SO₂R⁶)₂, and NR⁶(SO₂R⁶),
wherein R⁶ is in each case independently,
a cyclic radical,
C₁₋₈ alkyl, C₃₋₈ cyclo(hetero)alkyl,
C₂₋₈ alkenyl, C₃₋₈ cyclo(hetero)alkenyl,
C₂₋₈ alkynyl each optionally mono or polysubstituted with halo, OH and/or O—C₁₋₃ alkyl, and/or a cyclic radical,
R⁷, OR⁷, SR⁷, NHSO₂R⁷, N(SO₂R⁷)₂, or N(R⁸)SO₂R⁷,
wherein R⁷ is aryl, heteroaryl, aryl-C₁₋₅alkyl, heteroaryl-C₁₋₅ alkyl,
wherein aryl is phenyl or naphthyl, heteroaryl is an aromatic heterocyclic ring system of 5 to 15 ring atoms containing at least one atom selected from N including N-oxide, S, and O and wherein aryl and heteroaryl are optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical,
R⁸ is C₁₋₅ alkyl, optionally mono or polysubstituted with halo, OH, O—C₁₋₃ alkyl and/or a cyclic radical,
R⁴ is selected from
H,
halo,
a cyclic radical,
R⁹,
OH or OR⁹,
NH(C═O)—C₁₋₃ alkyl, optionally mono- or polysubstituted with halo, OH, O—C₁₋₃ alkyl and/or a cyclic radical or
NH₂, NHR⁹ or NR⁹R¹⁰,
wherein R⁹ and R¹⁰ are independently selected from
a cyclic radical,
C₁₋₆ alkyl or C₃₋₆ cyclo(hetero)alkyl, optionally mono- or polysubstituted with halo, OH, O—C₁₋₃ alkyl and/or a cyclic radical,
aryl-C₁₋₅-alkyl wherein aryl is phenyl, optionally mono- or polysubstituted with halo, amino, C₁₋₃ alkylamino, di-C₁₋₃ alkylamino, nitro, C₁₋₃ alkyl, OH, O—C₁₋₃ alkyl and/or a cyclic radical, or
NR⁹R¹⁰ together form a saturated or unsaturated five-, six- or seven-membered ring which can contain up to 3 heteroatoms, preferably N including N-oxide, S and/or O, optionally mono- or polysubstituted with halo, amino, C₁₋₃ alkylamino, di-C₁₋₃ alkylamino, C₁₋₃ alkyl, O—C₁₋₃ alkyl and/or aryl-C₁₋₅-alkyl, wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical,
R⁵ is selected from
H,
C₁₋₅ alkyl, C₃₋₆ cycloalkyl or (CO)—C₁₋₅ alkyl, optionally mono or polysubstituted with halo, OH, O—C₁₋₃ alkyl and/or a cyclic radical,
or pharmaceutically acceptable salts thereof.

25. The method of claim 24 wherein said patient is overweight or obese.

26. The method of claim 24 wherein the compound is a selective PDE10 inhibitor.

27. The method of claim 24 further comprising administering a further therapeutic agent.

28. The method of claim 27 wherein said further therapeutic agent is an anti-obesity agent.

29. The method of claim 24 wherein the bond between A and N is a double bond.

30. The method of claim 24 wherein m and n are both 0.

31. The method of claim 24 wherein R¹ is selected from
H,
C₁₋₄ alkyl, particularly C₂₋₄ alkyl optionally mono- or polysubstituted with halo, OH, O—C₁₋₃ alkyl and/or a cyclic radical or
phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical.

32. The method of claim 24 wherein R¹ is C₂₋₄-alkyl.

33. The method of claim 24 wherein R¹ is selected from n-propyl, i-propyl, or phenyl, each optionally substituted.

34. The method of claim 24 wherein R² is
H or
C₁₋₄ alkyl, particularly methyl, optionally substituted, e.g. halo substituted.

35. The method of claim 24 wherein R² is hydrogen, a methyl group, or a trifluoromethyl group.

36. The method of claim 24 wherein R³ is H, CN or C₁₋₃ alkyl.

37. The method of claim 24 wherein R³ is methyl.

38. The method of claim 24 wherein R³ is NH—(C═O)OR⁶

39. The method of claim 24 wherein R³ is NH—(C═O)—OC₁₋₅ alkyl, optionally mono- or polysubstituted.

40. The method of claim 24 wherein R³ is NH—SO₂R⁶.

41. The method of claim 24 wherein R³ is NH—SO₂—C₁₋₅ alkyl, optionally mono- or polysubstituted.

42. The method of claim 24 wherein R⁴ is selected from
H, C₁₋₃ alkyl, O—C₁₋₃ alkyl, NH₂, NHC₁₋₃ alkyl, wherein alkyl is optionally mono- or polysubstituted with halo, OH, O—C₁₋₃ alkyl and/or a cyclic radical or
NH(C═O)—C₁₋₃ alkyl, optionally mono- or polysubstituted with halo, OH, O—C₁₋₃ alkyl and/or a cyclic radical or
cyclopropyl, cyclobutyl, tetrahydropyrrolyl, pyrrolyl, pyrazolyl, imidazolyl, 1,2,3-triazolyl, 1,2,4-triazolyl, piperidinyl, morpholinyl, piperazinyl, optionally mono- or polysubstituted with halo, OH, C₁₋₅ alkyl and/or O—C₁₋₃ alkyl, or aryl-C₁₋₅-alkyl, wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical.

43. The method of claim 24 wherein R⁴ is H, C₁₋₃ alkyl or O—C₁₋₃ alkyl.

44. The method of claim 24 wherein H or OCH₃.

45. The method of claim 24 wherein said compound is selected from:
4,8-dimethoxy-3-methyl-1-propyl-imidazo[1,5-a]pyrido[3,2-e]pyrazine;
4,8-dimethoxy-1-propyl-imidazo[1,5-a]pyrido[3,2-e]pyrazine;
4,8-dimethoxy-1-ethyl-3-methyl-imidazo[1,5-a]pyrido[3,2-e]pyrazine;
4,8-dimethoxy-1,3-dimethyl-imidazo[1,5-a]pyrido[3,2-e]pyrazine;
4,8-dimethoxy-3-methyl-imidazo[1,5-a]pyrido[3,2-e]pyrazine;
1-ethyl-4-isopropyloxy-8-methoxy-3-methyl-imidazo[1,5-a]pyrido[3,2-e]pyrazine;
1-ethyl-8-methoxy-3-methyl-4-propyloxy-imidazo[1,5-a]pyrido[3,2-e]pyrazine;
4-cyclopentyloxy-1-ethyl-8-methoxy-3-methyl-imidazo[1,5-a]pyrido[3,2-e]pyrazine;
4-isopropyloxy-8-methoxy-3-methyl-1-propyl-imidazo[1,5-a]pyrido[3,2-e]pyrazine;
8-methoxy-1,3-dimethyl-4-(2,3,6-trifluorobenzyloxy)-imidazo[1,5-a]pyrido[3,2-e]pyrazine;
4-(2,4-dichlorobenzyloxy)-1-ethyl-8-methoxy-3-methyl-imidazo[1,5-a]pyrido[3,2-e]pyrazine;
4-(2-chloro-6-fluorobenzyloxy)-1-ethyl-8-methoxy-3-methyl-imidazo[1,5-a]pyrido[3,2-e]pyrazine;
1-ethyl-8-methoxy-3-methyl-4-(2,3,6-trifluorobenzyloxy)-imidazo[1,5-a]pyrido[3,2-e]pyrazine;
1-ethyl-8-methoxy-3-methyl-4-(2,4,6-trimethylbenzyloxy)-imidazo[1,5-a]pyrido[3,2-e]pyrazine;
4-(2-chloro-6-fluorobenzyloxy)-8-methoxy-3-methyl-1-propyl-imidazo[1,5-a]pyrido[3,2-e]pyrazine;
4-(2,6-difluorobenzyloxy)-8-methoxy-3-methyl-1-propyl-imidazo[1,5-a]pyrido[3,2-e]pyrazine;
1-ethyl-8-methoxy-3-methyl-4-(2-phenylethyloxy)-imidazo[1,5-a]pyrido[3,2-e]pyrazine;
8-methoxy-3-methyl-4-(2-phenylethyloxy)-1-propyl-imidazo[1,5-a]pyrido[3,2-e]pyrazine;
8-methoxy-1,3-dimethyl-4-(2-phenylethyloxy)-imidazo[1,5-a]pyrido[3,2-e]pyrazine;
8-methoxy-3-methyl-4-(2-phenylethyloxy)-imidazo[1,5-a]pyrido[3,2-e]pyrazine;
8-methoxy-3-methyl-4-(3-phenylpropyloxy)-1-propyl-imidazo[1,5-a]pyrido[3,2-e]pyrazine;
1-ethyl-8-methoxy-3-methyl-4-(3-phenylpropyloxy)-imidazo[1,5-a]pyrido[3,2-e]pyrazine;
1,3-dimethyl-8-methoxy-4-(3-phenylpropyloxy)-imidazo[1,5-a]pyrido[3,2-e]pyrazine;
4-[(3,5-dimethylisoxazol-4-yl)methyloxy]-1-ethyl-8-methoxy-3-methyl-imidazo[1,5-a]pyrido[3,2-e]pyrazine;
1-ethyl-8-methoxy-3-methyl-4-methylthio-imidazo[1,5-a]pyrido[3,2-e]pyrazine;
8-methoxy-3-methyl-4-methylthio-1-propyl-imidazo[1,5-a]pyrido[3,2-e]pyrazine;
1,3-dimethyl-8-methoxy-4-methylthio-imidazo[1,5-a]pyrido[3,2-e]pyrazine;
8-methoxy-3-methyl-4-methylthio-imidazo[1,5-a]pyrido[3,2-e]pyrazine;
4-cyano-8-methoxy-3-methyl-1-propyl-imidazo[1,5-a]pyrido[3,2-e]pyrazine;
4-cyano-8-methoxy-3-methyl-1-ethyl-imidazo[1,5-a]pyrido[3,2-e]pyrazine;
4-azido-8-methoxy-3-methyl-1-propyl-imidazo[1,5-a]pyrido[3,2-e]pyrazine;
8-methoxy-3-methyl-4-methylsulfinyl-1-propyl-imidazo[1,5-a]pyrido[3,2-e]pyrazine;
8-methoxy-3-methyl-4-methylsulfonyl-1-propyl-imidazo[1,5-a]pyrido[3,2-e]pyrazine;
1-ethyl-8-methoxy-3-methyl-4-methylsulfinyl-imidazo[1,5-a]pyrido[3,2-e]pyrazine;
8-methoxy-3-methyl-1-propyl-imidazo[1,5-a]pyrido[3,2-e]pyrazine;
1-ethyl-8-methoxy-3-methyl-imidazo[1,5-a]pyrido[3,2-e]pyrazine;
4-ethyl-8-methoxy-3-methyl-1-propyl-imidazo[1,5-a]pyrido[3,2-e]pyrazine;
3,4-dimethyl-8-methoxy-1-propyl-imidazo[1,5-a]pyrido[3,2-e]pyrazine;
3,4-dimethyl-8-methoxy-1-propyl-imidazo[1,5-a]pyrido[3,2-e]pyrazine hydrochloride;
1-ethyl-3,4-dimethyl-8-methoxy-imidazo[1,5-a]pyrido[3,2-e]pyrazine;
1,3,4-trimethyl-8-methoxy-imidazo[1,5-a]pyrido[3,2-e]pyrazine;
3,4-dimethyl-8-methoxy-1-(3,3,3-trifluoropropyl)-imidazo[1,5-a]pyrido[3,2-e]pyrazine;
3,4-dimethyl-8-methoxy-1-pentyl-imidazo[1,5-a]pyrido[3,2-e]pyrazine;
1-cyclohexyl-3,4-dimethyl-8-methoxy-imidazo[1,5-a]pyrido[3,2-e]pyrazine;
3,4-dimethyl-1-hexyl-8-methoxy-imidazo[1,5-a]pyrido[3,2-e]pyrazine;
3,4-dimethyl-8-methoxy-1-phenethyl-imidazo[1,5-a]pyrido[3,2-e]pyrazine;
3,4-dimethyl-8-methoxy-1-phenyl-imidazo[1,5-a]pyrido[3,2-e]pyrazine;
3,4-dimethyl-8-methoxy-1-phenyl-imidazo[1,5-a]pyrido[3,2-e]pyrazine dihydrochloride;
3,4-dimethyl-8-methoxy-1-(2-chlorophenyl)-imidazo[1,5-a]pyrido[3,2-e]pyrazine;
3,4-dimethyl-8-methoxy-1-(4-fluorophenyl)-imidazo[1,5-a]pyrido[3,2-e]pyrazine;
1-propyl-3,4,8-trimethyl-imidazo[1,5-a]pyrido[3,2-e]pyrazine;
1-propyl-3,4-dimethyl-imidazo[1,5-a]pyrido[3,2-e]pyrazine;
1-propyl-4,8-dimethyl-imidazo[1,5-a]pyrido[3,2-e]pyrazine;
8-difluoromethoxy-3,4-dimethyl-1-propyl-imidazo[1,5-a]pyrido[3,2-e]pyrazine;
3,4-dimethyl-8-(piperidin-1-yl)-methoxy-1-propyl-imidazo[1,5-a]pyrido[3,2-e]pyrazine;
3,4-dimethyl-8-(4-methyl-piperazin-1-yl)-methoxy-1-propyl-imidazo[1,5-a]pyrido[3,2-e]pyrazine;
3,4-dimethyl-8-(2-ethyl-4-methyl-imidazol-1-yl)-methoxy-1-propyl-imidazo[1,5-a]pyrido[3,2-e]pyrazine;
3,4-dimethyl-8-(2-propyl-4-methyl-imidazol-1-yl)-methoxy-1-propyl-imidazo[1,5-a]pyrido[3,2-e]pyrazine;
4-difluoromethoxy-3-methyl-1-propyl-imidazo[1,5-a]pyrido[3,2-e]pyrazine-8-ol;
8-methoxy-3-methyl-5-oxo-1-propyl-imidazo[1,5-a]pyrido[3,2-e]pyrazine;
3,4-dimethyl-8-methoxy-5-oxo-1-propyl-imidazo[1,5-a]pyrido[3,2-e]pyrazine;
8-methoxy-4-methoxycarbonylamino-3-methyl-1-propyl-imidazo[1,5-a]pyrido[3,2-e]pyrazine;
4-ethoxycarbonylamino-8-methoxy-3-methyl-1-propyl-imidazo[1,5-a]pyrido[3,2-e]pyrazine;
4-(N,N-bis-methoxycarbonyl)-amino-8-methoxy-3-methyl-1-propyl-imidazo[1,5-a]pyrido[3,2-e]pyrazine;
8-methoxy-4-(methoxycarbonyl-methyl-amino)-3-methyl-1-propyl-imidazo[1,5-a]pyrido[3,2-e]pyrazine;
8-methoxy-3-methyl-4-(3-methyl-ureido)-1-propyl-imidazo[1,5-a]pyrido[3,2-e]pyrazine;
8-methoxy-3-methyl-1-propyl-4-ureido-imidazo[1,5-a]pyrido[3,2-e]pyrazine;
8-methoxy-3-methyl-4-(3-isopropyl-ureido)-1-propyl-imidazo[1,5-a]pyrido[3,2-e]pyrazine;
8-methoxy-3-methyl-4-methylsulfonylamino-1-propyl-imidazo[1,5-a]pyrido[3,2-e]pyrazine;
4-(N,N-bis-methylsulfonyl)-amino-8-methoxy-3-methyl-1-propyl-imidazo[1,5-a]pyrido[3,2-e]pyrazine;
4-ethylsulfonylamino-8-methoxy-3-methyl-1-propyl-imidazo[1,5-a]pyrido[3,2-e]pyrazine;
1-ethyl-8-methoxy-3-methyl-4-methylsulfonylamino-imidazo[1,5-a]pyrido[3,2-e]pyrazine;
8-methoxy-3-methyl-1-propyl-4-trifluoromethylsulfonylamino-imidazo[1,5-a]pyrido[3,2-e]pyrazine;
8-methoxy-3-methyl-1-propyl-4-propylsulfonylamino-imidazo[1,5-a]pyrido[3,2-e]pyrazine;
4-isopropylsulfonylamino-8-methoxy-3-methyl-1-propyl-imidazo[1,5-a]pyrido[3,2-e]pyrazine;
8-methoxy-3-methyl-4-(4-methylphenylsulfonylamino)-1-propyl-imidazo[1,5-a]pyrido[3,2-e]pyrazine;
4-[N,N-bis-(4-methylphenylsulfonyl)-amino]-8-methoxy-3-methyl-1-propyl-imidazo[1,5-a]pyrido[3,2-e]pyrazine;
8-methoxy-3-methyl-1-(3,3,3-trifluoropropyl)-4-methylsulfonylamino-imidazo[1,5-a]pyrido[3,2-e]pyrazine;
1-hexyl-8-methoxy-3-methyl-4-methylsulfonylamino-imidazo[1,5-a]pyrido[3,2-e]pyrazine;
8-methoxy-3-methyl-1-phenethyl-4-methylsulfonylamino-imidazo[1,5-a]pyrido[3,2-e]pyrazine;
8-methoxy-3-methyl-1-phenyl-4-methylsulfonylamino-imidazo[1,5-a]pyrido[3,2-e]pyrazine;
1-(2-chlorophenyl)-8-methoxy-3-methyl-4-methylsulfonylamino-imidazo[1,5-a]pyrido[3,2-e]pyrazine;
1-(4-fluorophenyl)-8-methoxy-3-methyl-4-methylsulfonylamino-imidazo[1,5-a]pyrido[3,2-e]pyrazine;
3-methyl-8-(4-methyl-2-propyl-imidazol-1-yl)-1-propyl-4-methylsulfonylamino-imidazo[1,5-a]pyrido[3,2-e]pyrazine;
3-methyl-4-methylsulfonylamino-1-propyl-imidazo[1,5-a]pyrido[3,2-e]pyrazin-8-ol hydrobromide;
3-methyl-4-methylsulfonylamino-1-propyl-imidazo[1,5-a]pyrido[3,2-e]pyrazin-8-ol;
8-difluoromethoxy-3-methyl-4-methylsulfonylamino-1-propyl-imidazo[1,5-a]pyrido[3,2-e]pyrazine;
8-cyclopropylmethoxy-3-methyl-4-methylsulfonylamino-1-propyl-imidazo[1,5-a]pyrido[3,2-e]pyrazine;
3-methyl-1-propyl-4,5-dihydro-imidazo[1,5-a]pyrido[3,2-e]pyrazine;
8-methoxy-1-propyl-4,5-dihydro-imidazo[1,5-a]pyrido[3,2-e]pyrazine;
8-methoxy-3-methyl-1-propyl-4,5-dihydro-imidazo[1,5-a]pyrido[3,2-e]pyrazine;
8-methoxy-3-methyl-1-propyl-4,5-dihydro-imidazo[1,5-a]pyrido[3,2-e]pyrazine hydrochloride;
1-ethyl-8-methoxy-3-methyl-4,5-dihydro-imidazo[1,5-a]pyrido[3,2-e]pyrazine;
3,5-dimethyl-8-methoxy-1-propyl-4,5-dihydro-imidazo[1,5-a]pyrido[3,2-e]pyrazine;
5-acetyl-8-methoxy-3-methyl-1-propyl-4,5-dihydro-imidazo[1,5-a]pyrido[3,2-e]pyrazine;
and their pharmaceutically acceptable salts.

46. The method of claim 24 wherein said compound is 3,4-Dimethyl-8-methoxy-1-propyl-imidazo[1,5-a]-pyrido[3,2-e]-pyrazine or a pharmaceutically acceptable salt thereof.

47. A method of treating or preventing obesity, type 2 diabetes, metabolic syndrome, or glucose intolerance comprising administering to a patient in need a therapeutically effective amount of a compound of Formula (IIb): 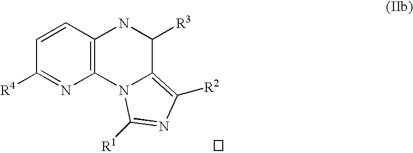
wherein R¹ and R² are independently selected from
H,
a cyclic radical,
C₁₋₈ alkyl or C₃₋₈ cycloalkyl, optionally mono- or polysubstituted with halo, OH, O—C₁₋₃ alkyl, and/or a cyclic radical,
C₂₋₈ alkenyl or C₃₋₈ cycloalkenyl, optionally mono- or polysubstituted with halo, OH, O—C₁₋₃ alkyl and/or a cyclic radical,
C₂-C₈ alkynyl, optionally mono- or polysubstituted with halo, OH, O—C₁₋₃-alkyl, and/or a cyclic radical,
a saturated, monounsaturated or polyunsaturated heterocycle with 5 to 15 ring atoms, optionally mono- or polysubstituted with halo, amino, C₁₋₃ alkylamino, di-C₁₋₃ alkylamino, nitro, C₁₋₃ alkyl, and/or O—C₁₋₃ alkyl, and
phenyl, optionally mono- or polysubstituted with halo, amino, C₁₋₃ alkylamino, di-C₁₋₃ alkylamino, nitro, C₁₋₃ alkyl, and/or OC₁₋₃ alkyl and/or a cyclic radical,
R³ is NH₂, NHR⁵ or NR⁵R⁶;
wherein R⁵ and R⁶ are independently selected from
a cyclic radical,
C₁₋₅ alkyl, optionally mono- or polysubstituted with halo, OH, O—C₁₋₃ alkyl and/or a cyclic radical,
aryl-C₁₋₅-alkyl wherein aryl is phenyl, optionally mono- or polysubstituted with halo, nitro, C₁₋₃ alkyl, OC₁₋₃ alkyl, and/or a cyclic radical,
(C═O)—C₁₋₅ alkyl optionally mono- or polysubstituted with halo, OH, O—C₁₋₃ alkyl and/or a cyclic radical,
or NR⁵R⁶ together form a saturated or unsaturated five-, six- or seven-membered ring which can contain up to 3 heteroatoms, preferably N including N-oxide, S and O, optionally mono- or polysubstituted with halo, C₁₋₃ alkyl, O—C₁₋₃ alkyl and/or aryl-C₁₋₅-alkyl, wherein aryl is phenyl, optionally mono- or polysubstituted with halo, nitro, C₁₋₃ alkyl, and/or O—C₁₋₃ alkyl, and/or a cyclic radical, and
R⁴ is selected from
H,
halo,
a cyclic radical,
R⁷,
OH or OR⁷,
NH(C═O)—C₁₋₃ alkyl, optionally mono- or polysubstituted with halo, OH, O—C₁₋₃ alkyl and/or a cyclic radical, in particular aryl or phenyl, or NH₂, NHR⁷ or NR⁷R⁸,
wherein R⁷ and R⁸ are independently selected from a cyclic radical,
C₁₋₆ alkyl or C₃₋₆ cycloalkyl, optionally mono- or polysubstituted with halo, OH, O—C₁₋₃ alkyl, and/or a cyclic radical,
aryl-C₁₋₅-alkyl wherein aryl is phenyl, optionally mono- or polysubstituted with halo, nitro, C₁₋₃ alkyl, O—C₁₋₃ alkyl, and/or a cyclic radical,
or NR⁷R⁸ together form a saturated or unsaturated five- or six-membered ring which can contain up to 3 heteroatoms, preferably N including N-oxide, S and O, optionally mono- or polysubstituted with halo, C₁₋₃ alkyl, C₃₋₆ cycloalkyl, O—C₁₋₃ alkyl and/or aryl-C₁₋₅-alkyl, wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical,
or pharmaceutically acceptable salt thereof.

48. The method of claim 47 wherein said patient is overweight or obese.

49. The method of claim 47 wherein the compound is a selective PDE10 inhibitor.

50. The method of claim 47 further comprising administering a further therapeutic agent.

51. The method of claim 50 wherein said further therapeutic agent is an anti-obesity agent.

52. The method of claim 47 wherein R¹ is selected from
H,
C₁₋₄ alkyl, particularly C₂₋₄ alkyl optionally mono- or polysubstituted with halo, OH, C₁₋₃ alkyl, or/and a cyclic radical or
phenyl, optionally mono- or polysubstituted with halo, amino, C₁₋₃ alkylamino, di-C₁₋₃ alkylamino, nitro, C₁₋₃ alkyl, O—C₁₋₃ alkyl or/and a cyclic radical.

53. The method of claim 47 wherein R¹ is C₂₋₄-alkyl or phenyl.

54. The method of claim 47 wherein R² is
H or C₁₋₄ alkyl optionally halogenated, particularly methyl or trifluoromethyl.

55. The method of claim 47 wherein R² is hydrogen or a methyl group.

56. The method of claim 47 wherein R³ is selected from
NH₂,
NHC₁₋₃ alkyl, optionally mono- or polysubstituted with halo, OH, O—C₁₋₃ alkyl and/or a cyclic radical, or
NH(C═O)—C₁₋₃ alkyl, optionally mono- or polysubstituted with halo, OH, O—C₁₋₃ alkyl and/or a cyclic radical or
cyclopropyl, cyclobutyl, tetrahydropyrrolyl, pyrrolyl, pyrazolyl, imidazolyl, 1,2,3-triazolyl, 1,2,4-triazolyl, piperidinyl, morpholinyl, piperazinyl, optionally substituted with C₁₋₃ alkyl, optionally mono- or polysubstituted with halo, OH and/or O—C₁₋₃ alkyl, or arylalkyl, wherein aryl is phenyl, optionally mono- or polysubstituted with halo, amino, C₁₋₃ alkylamino, di-C₁₋₃ alkylamino, nitro, C₁₋₃ alkyl, and/or O—C₁₋₃ alkyl and/or a cyclic radical.

57. The method of claim 1 wherein R³ is —NH₂, —NH—C₁₋₃-alkyl, —NH—(C═O)—C₁₋₃-alkyl or -imidazolyl.

58. The method of claim 1 wherein R⁴ is selected from
OH or O—C₁₋₃ alkyl, optionally mono- or polysubstituted with halo, OH, O—C₁₋₃ alkyl, and/or a cyclic radical, NHC₁₋₃ alkyl, optionally mono- or polysubstituted with halo, OH, O—C₁₋₃ alkyl and/or a cyclic radical, or
NH benzyl, wherein the phenyl group is phenyl, optionally mono- or polysubstituted with halo, amino, C₁₋₃ alkylamino, di-C₁₋₃ alkylamino, nitro, C₁₋₃ alkyl, O—C₁₋₃ alkyl or a cyclic radical or
cyclopropyl, cyclobutyl, tetrahydropyrrolyl, pyrrolyl, pyrazolyl, imidazolyl, 1,2,3-triazolyl, 1,2,4-triazolyl, piperidinyl, morpholinyl, piperazinyl, optionally substituted with C₁₋₃ alkyl, optionally mono- or polysubstituted with halo, OH, C₁₋₅ alkyl and/or O—C₁₋₃ alkyl, or arylalkyl, wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical.

59. The method of claim 1 wherein said compound is selected from:
4-amino-8-methoxy-3-methyl-1-propyl-imidazo[1,5-a]pyrido[3,2-e]pyrazine;
4-amino-1-ethyl-8-methoxy-3-methyl-imidazo[1,5-a]pyrido[3,2-e]pyrazine;
4-amino-1-ethyl-3-methyl-imidazo[1,5-a]pyrido[3,2-e]pyrazine;
4-amino-3-methyl-1-propyl-imidazo[1,5-a]pyrido[3,2-e]pyrazine;
4-amino-1-ethyl-8-(2-ethyl-4-methyl-imidazol-1-yl)-3-methyl-imidazo[1,5-a]pyrido[3,2-e]pyrazine;
4-amino-3-methyl-1-propyll-8-(2-propyl-4-methyl-imidazol-1-yl)-imidazo[1,5-a]pyrido[3,2-e]pyrazine;
4-amino-1-hexyl-8-methoxy-3-methyl-imidazo[1,5-a]pyrido[3,2-e]pyrazine;
4-amino-8-methoxy-3-methyl-1-(3,3,3-trifluoropropyl)-imidazo[1,5-a]pyrido[3,2-e]pyrazine;
4-amino-8-methoxy-3-methyl-1-phenethyl-imidazo[1,5-a]pyrido[3,2-e]pyrazine;
4-amino-8-methoxy-3-methyl-1-phenyl-imidazo[1,5-a]pyrido[3,2-e]pyrazine;
4-amino-1-(2-chloro-phenyl)-8-methoxy-3-methyl-imidazo[1,5-a]pyrido[3,2-e]pyrazine;
4-amino-1-(4-fluoro-phenyl)-8-methoxy-3-methyl-imidazo[1,5-a]pyrido[3,2-e]pyrazine;
4-amino-1-isopropyl-8-methoxy-imidazo[1,5-a]pyrido[3,2-e]pyrazine;
4-amino-8-methoxy-imidazo[1,5-a]pyrido[3,2-e]pyrazine;
4-amino-8-methoxy-3-phenyl-imidazo[1,5-a]pyrido[3,2-e]pyrazine;
4-(N-methyl-amino)-8-methoxy-3-methyl-1-propyl-imidazo[1,5-a]pyrido[3,2-e]pyrazine;
4-(N-ethyl-amino)-8-methoxy-3-methyl-1-propyl-imidazo[1,5-a]pyrido[3,2-e]pyrazine;
4-(N-methyl-amino)-1-ethyl-8-methoxy-3-methyl-imidazo[1,5-a]pyrido[3,2-e]pyrazine;
4-(N,N-dimethyl-amino)-8-methoxy-3-methyl-1-propyl-imidazo[1,5-a]pyrido[3,2-e]pyrazine;
4-(N-butyl-amino)-1-ethyl-8-methoxy-3-methyl-imidazo[1,5-a]pyrido[3,2-e]pyrazine;
4-(N-benzyl-amino)-1-ethyl-8-methoxy-3-methyl-imidazo[1,5-a]pyrido[3,2-e]pyrazine;
4-(N-cyclopentyl-amino)-1-ethyl-8-methoxy-3-methyl-imidazo[1,5-a]pyrido[3,2-e]pyrazine;
4-(N-cyclopentyl-amino)-8-methoxy-3-methyl-1-propyl-imidazo[1,5-a]pyrido[3,2-e]pyrazine;
1-ethyl-8-methoxy-3-methyl-4-morpholino-imidazo[1,5-a]pyrido[3,2-e]pyrazine;
4-azetidine-8-methoxy-3-methyl-1-(3,3,3-trifluoropropyl)-imidazo[1,5-a]pyrido[3,2-e]pyrazine;
8-methoxy-3-methyl-1-propyl-4-pyrrolidino-imidazo[1,5-a]pyrido[3,2-e]pyrazine;
8-methoxy-3-methyl-4-piperidino-1-propyl-imidazo[1,5-a]pyrido[3,2-e]pyrazine;
1-ethyl-8-methoxy-3-methyl-4-(4-phenylpiperazino)-imidazo[1,5-a]pyrido[3,2-e]pyrazine;
8-methoxy-3-methyl-1-propyl-4-(pyrazol-1-yl)-imidazo[1,5-a]pyrido[3,2-e]pyrazine;
8-methoxy-3-methyl-1-propyl-4-(pyrazol-1-yl)-imidazo[1,5-a]pyrido[3,2-e]pyrazine hydro chloride;
4-(imidazol-1-yl)-8-methoxy-3-methyl-1-propyl-imidazo[1,5-a]pyrido[3,2-e]pyrazine;
8-methoxy-3-methyl-1-propyl-4-(1,2,3-triazol-1-yl)-imidazo[1,5-a]pyrido[3,2-e]pyrazine;
8-methoxy-3-methyl-1-propyl-4-(1,2,4-triazol-1-yl)-imidazo[1,5-a]pyrido[3,2-e]pyrazine;
8-methoxy-3-methyl-4-(2-methyl-imidazol-1-yl)-1-propyl-imidazo[1,5-a]pyrido[3,2-e]pyrazine;
4-(imidazol-1-yl)-3-methyl-1-propyl-imidazo[1,5-a]pyrido[3,2-e]pyrazine-8-ol;
1-ethyl-4-(N-formyl-amino)-8-methoxy-3-methyl-imidazo[1,5-a]pyrido[3,2-e]pyrazine;
4-(N-formyl-amino)-8-methoxy-3-methyl-1-propyl-imidazo[1,5-a]pyrido[3,2-e]pyrazine;
4-(N-acetyl-amino)-8-methoxy-3-methyl-1-propyl-imidazo[1,5-a]pyrido[3,2-e]pyrazine;
4-(N,N-diacetyl-amino)-8-methoxy-3-methyl-1-propyl-imidazo[1,5-a]pyrido[3,2-e]pyrazine;
4-(N-acetyl-amino)-1-ethyl-8-methoxy-3-methyl-imidazo[1,5-a]pyrido[3,2-e]pyrazine;
4-(N,N-diacetyl-amino)-1-ethyl-8-methoxy-3-methyl-imidazo[1,5-a]pyrido[3,2-e]pyrazine;
4-(N-acetyl-amino)-8-methoxy-3-methyl-1-phenyl-imidazo[1,5-a]pyrido[3,2-e]pyrazine;
8-methoxy-3-methyl-4-(N-propionyl-amino)-1-propyl-imidazo[1,5-a]pyrido[3,2-e]pyrazine;
4-(N-cyclopropylcarboxy-amino)-8-methoxy-3-methyl-1-propyl-imidazo[1,5-a]pyrido[3,2-e]pyrazine;
and pharmaceutically acceptable salts thereof.

60. A method of reducing body fat or body weight in a patient comprising administering to said patient in need a therapeutically effective amount of a compound of formula (IIb): 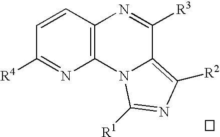
wherein R¹ and R² are independently selected from
H,
a cyclic radical,
C₁₋₈ alkyl or C₃₋₈ cycloalkyl, optionally mono- or polysubstituted with halo, OH, O—C₁₋₃ alkyl, and/or a cyclic radical,
C₂₋₈ alkenyl or C₃₋₈ cycloalkenyl, optionally mono- or polysubstituted with halo, OH, O—C₁₋₃ alkyl and/or a cyclic radical,
C₂-C₈ alkynyl, optionally mono- or polysubstituted with halo, OH, O—C₁₋₃-alkyl, and/or a cyclic radical,
a saturated, monounsaturated or polyunsaturated heterocycle with 5 to 15 ring atoms, optionally mono- or polysubstituted with halo, amino, C₁₋₃ alkylamino, di-C₁₋₃ alkylamino, nitro, C₁₋₃ alkyl, and/or O—C₁₋₃ alkyl, and
phenyl, optionally mono- or polysubstituted with halo, amino, C₁₋₃ alkylamino, di-C₁₋₃ alkylamino, nitro, C₁₋₃ alkyl, and/or OC₁₋₃ alkyl and/or a cyclic radical,
R³ is NH₂, NHR⁵ or NR⁵R⁶;
wherein R⁵ and R⁶ are independently selected from a cyclic radical,
C₁₋₅ alkyl, optionally mono- or polysubstituted with halo, OH, O—C₁₋₃ alkyl and/or a cyclic radical,
aryl-C₁₋₅-alkyl wherein aryl is phenyl, optionally mono- or polysubstituted with halo, nitro, C₁₋₃ alkyl, OC₁₋₃ alkyl, and/or a cyclic radical,
(C═O)—C₁₋₅ alkyl optionally mono- or polysubstituted with halo, OH, O—C₁₋₃ alkyl and/or a cyclic radical,
or NR⁵R⁶ together form a saturated or unsaturated five-, six- or seven-membered ring which can contain up to 3 heteroatoms, preferably N including N-oxide, S and O, optionally mono- or polysubstituted with halo, C₁₋₃ alkyl, O—C₁₋₃ alkyl and/or aryl-C₁₋₅-alkyl, wherein aryl is phenyl, optionally mono- or polysubstituted with halo, nitro, C₁₋₃ alkyl, and/or O—C₁₋₃ alkyl, and/or a cyclic radical, and
R⁴ is selected from
H,
halo,
a cyclic radical,
R⁷,
OH or OR⁷,
NH(C═O)—C₁₋₃ alkyl, optionally mono- or polysubstituted with halo, OH, O—C₁₋₃ alkyl and/or a cyclic radical, in particular aryl or phenyl, or
NH₂, NHR⁷ or NR⁷R⁸,
wherein R⁷ and R⁸ are independently selected from
a cyclic radical,
C₁₋₆ alkyl or C₃₋₆ cycloalkyl, optionally mono- or polysubstituted with halo, OH, O—C₁₋₃ alkyl, and/or a cyclic radical,
aryl-C₁₋₅-alkyl wherein aryl is phenyl, optionally mono- or polysubstituted with halo, nitro, C₁₋₃ alkyl, O—C₁₋₃ alkyl, and/or a cyclic radical,
or NR⁷R⁸ together form a saturated or unsaturated five- or six-membered ring which can contain up to 3 heteroatoms, preferably N including N-oxide, S and O, optionally mono- or polysubstituted with halo, C₁₋₃ alkyl, C₃₋₆ cycloalkyl, O—C₁₋₃ alkyl and/or aryl-C₁₋₅-alkyl, wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical,
or pharmaceutically acceptable salt thereof.

61. The method of claim 60 wherein said patient is overweight or obese.

62. The method of claim 60 wherein the compound is a selective PDE10 inhibitor.

63. The method of claim 60 further comprising administering a further therapeutic agent.

64. The method of claim 63 wherein said further therapeutic agent is an anti-obesity agent.

65. The method of claim 60 wherein R¹ is selected from
H,
C₁₋₄ alkyl, particularly C₂₋₄ alkyl optionally mono- or polysubstituted with halo, OH, C₁₋₃ alkyl, or/and a cyclic radical or
phenyl, optionally mono- or polysubstituted with halo, amino, C₁₋₃ alkylamino, di-C₁₋₃ alkylamino, nitro, C₁₋₃ alkyl, O—C₁₋₃ alkyl or/and a cyclic radical.

66. The method of claim 60 wherein R¹ is C₂₋₄-alkyl or phenyl.

67. The method of claim 60 wherein R² is H or C₁₋₄ alkyl optionally halogenated, particularly methyl or trifluoromethyl.

68. The method of claim 60 wherein R² is hydrogen or a methyl group.

69. The method of claim 60 wherein R³ is selected from
NH₂,
NHC₁₋₃ alkyl, optionally mono- or polysubstituted with halo, OH, O—C₁₋₃ alkyl and/or a cyclic radical, or
NH(C═O)—C₁₋₃ alkyl, optionally mono- or polysubstituted with halo, OH, O—C₁₋₃ alkyl and/or a cyclic radical or
cyclopropyl, cyclobutyl, tetrahydropyrrolyl, pyrrolyl, pyrazolyl, imidazolyl, 1,2,3-triazolyl, 1,2,4-triazolyl, piperidinyl, morpholinyl, piperazinyl, optionally substituted with C₁₋₃ alkyl, optionally mono- or polysubstituted with halo, OH and/or O—C₁₋₃ alkyl, or arylalkyl, wherein aryl is phenyl, optionally mono- or polysubstituted with halo, amino, C₁₋₃ alkylamino, di-C₁₋₃ alkylamino, nitro, C₁₋₃ alkyl, and/or O—C₁₋₃ alkyl and/or a cyclic radical.

70. The method of claim 60 wherein R³ is —NH₂, —NH—C₁₋₃-alkyl, —NH—(C═O)—C₁₋₃-alkyl or -imidazolyl.

71. The method of claim 60 wherein R⁴ is selected from
OH or O—C₁₋₃ alkyl, optionally mono- or polysubstituted with halo, OH, O—C₁₋₃ alkyl, and/or a cyclic radical,
NHC₁₋₃ alkyl, optionally mono- or polysubstituted with halo, OH, O—C₁₋₃ alkyl and/or a cyclic radical, or
NH benzyl, wherein the phenyl group is phenyl, optionally mono- or polysubstituted with halo, amino, C₁₋₃ alkylamino, di-C₁₋₃ alkylamino, nitro,
C₁₋₃ alkyl, O—C₁₋₃ alkyl or a cyclic radical or
cyclopropyl, cyclobutyl, tetrahydropyrrolyl, pyrrolyl, pyrazolyl, imidazolyl, 1,2,3-triazolyl, 1,2,4-triazolyl, piperidinyl, morpholinyl, piperazinyl, optionally substituted with C₁₋₃ alkyl, optionally mono- or polysubstituted with halo, OH, C₁₋₅ alkyl and/or O—C₁₋₃ alkyl, or arylalkyl, wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical.

72. The method of claim 60 wherein said compound is selected from:
4-amino-8-methoxy-3-methyl-1-propyl-imidazo[1,5-a]pyrido[3,2-e]pyrazine;
4-amino-1-ethyl-8-methoxy-3-methyl-imidazo[1,5-a]pyrido[3,2-e]pyrazine;
4-amino-1-ethyl-3-methyl-imidazo[1,5-a]pyrido[3,2-e]pyrazine;
4-amino-3-methyl-1-propyl-imidazo[1,5-a]pyrido[3,2-e]pyrazine;
4-amino-1-ethyl-8-(2-ethyl-4-methyl-imidazol-1-yl)-3-methyl-imidazo[1,5-a]pyrido[3,2-e]pyrazine;
4-amino-3-methyl-1-propyll-8-(2-propyl-4-methyl-imidazol-1-yl)-imidazo[1,5-a]pyrido[3,2-e]pyrazine;
4-amino-1-hexyl-8-methoxy-3-methyl-imidazo[1,5-a]pyrido[3,2-e]pyrazine;
4-amino-8-methoxy-3-methyl-1-(3,3,3-trifluoropropyl)-imidazo[1,5-a]pyrido[3,2-e]pyrazine;
4-amino-8-methoxy-3-methyl-1-phenethyl-imidazo[1,5-a]pyrido[3,2-e]pyrazine;
4-amino-8-methoxy-3-methyl-1-phenyl-imidazo[1,5-a]pyrido[3,2-e]pyrazine;
4-amino-1-(2-chloro-phenyl)-8-methoxy-3-methyl-imidazo[1,5-a]pyrido[3,2-e]pyrazine;
4-amino-1-(4-fluoro-phenyl)-8-methoxy-3-methyl-imidazo[1,5-a]pyrido[3,2-e]pyrazine;
4-amino-1-isopropyl-8-methoxy-imidazo[1,5-a]pyrido[3,2-e]pyrazine;
4-amino-8-methoxy-imidazo[1,5-a]pyrido[3,2-e]pyrazine;
4-amino-8-methoxy-3-phenyl-imidazo[1,5-a]pyrido[3,2-e]pyrazine;
4-(N-methyl-amino)-8-methoxy-3-methyl-1-propyl-imidazo[1,5-a]pyrido[3,2-e]pyrazine;
4-(N-ethyl-amino)-8-methoxy-3-methyl-1-propyl-imidazo[1,5-a]pyrido[3,2-e]pyrazine;
4-(N-methyl-amino)-1-ethyl-8-methoxy-3-methyl-imidazo[1,5-a]pyrido[3,2-e]pyrazine;
4-(N,N-dimethyl-amino)-8-methoxy-3-methyl-1-propyl-imidazo[1,5-a]pyrido[3,2-e]pyrazine;
4-(N-butyl-amino)-1-ethyl-8-methoxy-3-methyl-imidazo[1,5-a]pyrido[3,2-e]pyrazine;
4-(N-benzyl-amino)-1-ethyl-8-methoxy-3-methyl-imidazo[1,5-a]pyrido[3,2-e]pyrazine;
4-(N-cyclopentyl-amino)-1-ethyl-8-methoxy-3-methyl-imidazo[1,5-a]pyrido[3,2-e]pyrazine;
4-(N-cyclopentyl-amino)-8-methoxy-3-methyl-1-propyl-imidazo[1,5-a]pyrido[3,2-e]pyrazine;
1-ethyl-8-methoxy-3-methyl-4-morpholino-imidazo[1,5-a]pyrido[3,2-e]pyrazine;
4-azetidine-8-methoxy-3-methyl-1-(3,3,3-trifluoropropyl)-imidazo[1,5-a]pyrido[3,2-e]pyrazine;
8-methoxy-3-methyl-1-propyl-4-pyrrolidino-imidazo[1,5-a]pyrido[3,2-e]pyrazine;
8-methoxy-3-methyl-4-piperidino-1-propyl-imidazo[1,5-a]pyrido[3,2-e]pyrazine;
1-ethyl-8-methoxy-3-methyl-4-(4-phenylpiperazino)-imidazo[1,5-a]pyrido[3,2-e]pyrazine;
8-methoxy-3-methyl-1-propyl-4-(pyrazol-1-yl)-imidazo[1,5-a]pyrido[3,2-e]pyrazine;
8-methoxy-3-methyl-1-propyl-4-(pyrazol-1-yl)-imidazo[1,5-a]pyrido[3,2-e]pyrazine hydro chloride;
4-(imidazol-1-yl)-8-methoxy-3-methyl-1-propyl-imidazo[1,5-a]pyrido[3,2-e]pyrazine;
8-methoxy-3-methyl-1-propyl-4-(1,2,3-triazol-1-yl)-imidazo[1,5-a]pyrido[3,2-e]pyrazine;
8-methoxy-3-methyl-1-propyl-4-(1,2,4-triazol-1-yl)-imidazo[1,5-a]pyrido[3,2-e]pyrazine;
8-methoxy-3-methyl-4-(2-methyl-imidazol-1-yl)-1-propyl-imidazo[1,5-a]pyrido[3,2-e]pyrazine;
4-(imidazol-1-yl)-3-methyl-1-propyl-imidazo[1,5-a]pyrido[3,2-e]pyrazine-8-ol;
1-ethyl-4-(N-formyl-amino)-8-methoxy-3-methyl-imidazo[1,5-a]pyrido[3,2-e]pyrazine;
4-(N-formyl-amino)-8-methoxy-3-methyl-1-propyl-imidazo[1,5-a]pyrido[3,2-e]pyrazine;
4-(N-acetyl-amino)-8-methoxy-3-methyl-1-propyl-imidazo[1,5-a]pyrido[3,2-e]pyrazine;
4-(N,N-diacetyl-amino)-8-methoxy-3-methyl-1-propyl-imidazo[1,5-a]pyrido[3,2-e]pyrazine;
4-(N-acetyl-amino)-1-ethyl-8-methoxy-3-methyl-imidazo[1,5-a]pyrido[3,2-e]pyrazine;
4-(N,N-diacetyl-amino)-1-ethyl-8-methoxy-3-methyl-imidazo[1,5-a]pyrido[3,2-e]pyrazine;
4-(N-acetyl-amino)-8-methoxy-3-methyl-1-phenyl-imidazo[1,5-a]pyrido[3,2-e]pyrazine;
8-methoxy-3-methyl-4-(N-propionyl-amino)-1-propyl-imidazo[1,5-a]pyrido[3,2-e]pyrazine;
4-(N-cyclopropylcarboxy-amino)-8-methoxy-3-methyl-1-propyl-imidazo[1,5-a]pyrido[3,2-e]pyrazine;
and pharmaceutically acceptable salts thereof.


SEQUENCE LISTING (14 sequences)

SEQ ID NO: 1 - DNA, 26 bp, Artificial Sequence
  Primer
tgcatctacagggttaccatggagaa

SEQ ID NO: 2 - DNA, 29 bp, Artificial Sequence
  Primer
tatccctgcaggccttcagcagaggctct

SEQ ID NO: 3 - DNA, 28 bp, Artificial Sequence
  Primer
ttcacatggatatgcgacggtaccttct

SEQ ID NO: 4 - DNA, 31 bp, Artificial Sequence
  Primer
ctgtgaagaagaactatcggcgggttcctta

SEQ ID NO: 5 - DNA, 967 bp, Sus scrofa
tgcatctacagggttaccatggagaagctgtcctaccacagcatttgtaccgcggaagagtggcaaggcctcatgcgcttcaaccttcccgtccgtctttgcaaggagattgaattgttccacttcgacattggtccttttgaaaacatgtggcctggaatctttgtctatatggttcatcgcttctgtgggacggcctgctttgagcttgaaaagctgtgtcgttttatcatgtctgtgaagaagaactatcgtcgggttccttaccacaactggaagcacgcggtcacggtggcacactgcatgtacgccatcctccagaacagccacgggctcttcaccgacctcgagcgcaaaggactgctaatcgcgtgtctgtgccacgacctggaccacaggggcttcagcaacagctacctgcagaaattcgaccaccccctggccgctctctactccacgcccaccatggagcagcaccacttctcccagaccgtgtccatcctccagttggaagggcacaacatcttctccaccctgagctccagtgagtacgagcaggtgcttgagatcatccgcaaagccatcattgccacagacctcgctttgtactttggaaacaggaaacagttggaggagatgtaccagaccggatcgctaaaccttaataaccagtcacatagagaccgcgtcattggtttgatgatgactgcctgtgatctctgttccgtgacaaaactgtggccagtaacaaaactgacggcaaatgatatatatgcggaattctgggccgagggcgatgaggtgaagaagctgggaatacagcctattcccatgatggacagagacaagaaggacgaagtcccacaaggccagctcggattctacaacgcggtagctatcccctgctacaccaccctcacccagatcttcccgcccacagagcctcttctgaaggcctgcagggata

SEQ ID NO: 6 - DNA, 732 bp, Guinea pig
ctgtgaagaagaactatcggcgggttccttaccacaactggaagcatgcagtcacggtggcgcactgcatgtacgccatacttcaaaacaacaatggcctcttcacagaccttgagcgcaaaggcctgctaattgcctgtctgtgccatgacctggaccacaggggcttcagtaacagctacctgcagaaattcgaccaccccctggctgcgttgtactccacctccaccatggagcaacaccacttctcccagacggtgttcatcctccagctggaaggacacaacatcttctccaccctgagctccagcgagtacgagcaggtgctggagatcatccgcaaagccatcatcgccactgacctcgcactgtactttgggaacaggaagcagttggaggagatgtaccagacagggtcgctgaacctcaataaccagtcccatcgagaccgcgtcatcggcttgatgatgactgcctgcgatctttgctctgtgacgaaactatggccagttacaaaattgacagcaaatgatatatatgcagagttctgggctgagggggatgagatgaagaagttggggatacagcccatccctatgatggacagagacaagaaggatgaagtccctcaaggacagcttggattctacaatgctgtggccatcccctgctataccaccctgacgcagatcctcccacccacagagcctctgctgaaggcctgcagggata

SEQ ID NO: 7 - DNA, 266 bp, Artificial Sequence
  Synthetically generated oligonucleotide
tagagcctctgctgaaggcctgcagggataacctcaatcagtgggagaaggtaattcgaggggaagagacagcaatgtggatttcaggcccagcaactagcaaaagcacatcagggaagccgaccaggaaggtcgatgactgatcctgaggtgatgtctgcctagcaactgactcaacctgcttctgtgacttcgttctttttatttttatttttttaacggggtgaaaacctctctcagaaggtaccgtcgcatatccatgtgaa

SEQ ID NO: 8 - DNA, 993 bp, Artificial Sequence
  Consensus sequence
acccgagatggcaaggcctcatgccttcaactccgcgttgcggaatgatttccacttgacattggtcctttgaaacatgtggcctggatctttgtctaatgtcatcgttgtgggaccctgttttgacttgaaaatgtgcttttatcatgtctgtgaagaagaactatcggcgggttccttaccacaactggaagcatgcagtcacggtggcgcactgcatgtacgccatacttcaaaacaacaatggcctcttcacagaccttgagcgcaaaggcctgctaattgcctgtctgtgccatgacctggaccacaggggcttcagtaacagctacctgcagaaattcgaccaccccctggctgcgttgtactccacctccaccatggagcaacaccacttctcccagacggtgtccatcctccagctggaaggacacaacatcttctccaccctgagctccagcgagtacgagcaggtgctggagatcatccgcaaagccatcatcgccactgacctcgcactgtactttgggaacaggaagcagttggaggagatgtaccagacagggtcgctgaacctccacaaccagtcccatcgagaccgcgtcatcggcttgatgatgactgcctgcgatctttgctctgtgacgaaactatggccagttacaaaattgacagcaaatgatatatatgcagagttctgggctgagggggatgagatgaagaagttggggatacagcccatccctatgatggacagagacaagcgagatgaagtccctcaaggacagcttggattctacaatgctgtggccatcccctgctataccaccctgacgcagatcctcccacccacagagcctctgctgaaggcctgcagggataacctcaatcagtgggagaaggtaattcgaggggaagagacagcaatgtggatttcaggcccagcaactagcaaaagcacatcggaagccgaccaggaaggtcgatgactga

SEQ ID NO: 9 - DNA, 845 bp, Guinea pig
ctgtgaagaagaactatcgtcgggttccttaccacaactggaagcacgcggtcacggtggcacactgcatgtacgccatcctccagaacagccacgggctcttcaccgacctcgagcgcaaaggactgctaatcgcgtgtctgtgccacgacctggaccacaggggcttcagcaacagctacctgcagaaattcgaccaccccctggccgctctctactccacgcccaccatggagcagcaccacttctcccagaccgtgtccatcctccagttggaagggcacaacatcttctccaccctgagctccagtgagtacgagcaggtgcttgagatcatccgcaaagccatcattgccacagacctcgctttgtactttggaaacaggaaacagttggaggagatgtaccagaccggatcgctaaaccttaataaccagtcacatagagaccgcgtcattggtttgatgatgactgcctgtgatctctgttccgtgacaaaactgtggccagtaacaaaactgacggcaaatgatatatatgcggaattctgggccgagggcgatgaggtgaagaagctgggaatacagcctattcccatgatggacagagacaagaaggacgaagtcccacaaggccagctcggattctacaacgcggtagctatcccctgctacaccaccctcacccagatcttcccgcccacagagcctcttctgaaggcctgcagggataacctcaatcagtgggagaaggtaattcgaggggaagagacagcaatgtggatttcaggcccagcaactagcaaaagcacatcagggaagccgaccaggaaggtcgatgactga

SEQ ID NO: 10 - DNA, 1032 bp, Rattus norvegicus
acctctgaggaatggcaaggcctcatgcacttcaacttgccagcacgcatctgccgggacatcgagctattccactttgacattggtcctttcgagaacatgtggcctgggatctttgtctacatgatccatcggtcttgtgggacatcctgttttgaacttgaaaaattgtgccgttttatcatgtctgtgaagaagaactataggcgggttccttaccacaactggaagcatgcagtcacggtggcgcactgcatgtacgccatacttcaaaacaacaatggcctcttcacagaccttgagcgcaaaggcctgctaattgcctgtctgtgccatgacctggaccacaggggcttcagtaacagctacctgcagaaattcgaccaccccctggctgcgttgtactccacctccaccatggagcaacaccacttctcccagacggtgtccatcctccagctggaaggacacaacatcttctccaccctgagctccagcgagtacgagcaggtgctggagatcatccgcaaagccatcatcgccactgacctcgcactgtactttgggaacaggaagcagttggaggagatgtaccagacagggtcgctgaacctccacaaccagtcccatcgagaccgcgtcatcggcttgatgatgactgcctgcgatctttgctctgtgacgaaactatggccagttacaaaattgacagcaaatgatatatatgcagagttctgggctgagggggatgagatgaagaagttggggatacagcccatccctatgatggacagagacaagcgagatgaagtccctcaaggacagcttggattctacaatgctgtggccatcccctgctataccaccctgacgcagatcctcccacccacagagcctctgctgaaggcctgcagggataacctcaatcagtgggagaaggtaattcgaggggaagagacagcaatgtggatttcaggcccagcaactagcaaaagcacatctgagaagccgaccaggaaggtcgatgactga

SEQ ID NO: 11 - protein, 329 aa, Sus scrofa
IRLCIYRVTMEKLSYHSICTAEEWQGLMRFNLPVRLCKEIELFHFDIGPFENMWPGIFVYMVHRFCGTACFELEKLCRFIMSVKKNYRRVPYHNWKHAVTVAHCMYAILQNSHGLFTDLERKGLLIACLCHDLDHRGFSNSYLQKFDHPLAALYSTPTMEQHHFSQTVSILQLEGHNIFSTLSSSEYEQVLEIIRKAIIATDLALYFGNRKQLEEMYQTGSLNLNNQSHRDRVIGLMMTACDLCSVTKLWPVTKLTANDTYAEPWAEGDEVKKLGIQPIPMMDRDKKDEVPQGQLGFYNAVAIPCYTTLTQIFPPTEPLLKACRDKAEF

SEQ ID NO: 12 - protein, 280 aa, Guinea pig
VKKNYRRVPYHNWKHAVTVAHCMYAILQNSHGLFTDLERKGLLIACLCHDLDHRGFSNSYLQKFDHPLAALYSTSTMEQHHFSQTVFILQLEGHNIFSTLSSSEYEQVLEIIRKAIIATDLALYFGNRKQLEEMYQTGSLNLNNQSHRDRVIGLMMTACDLCSVTKLWPVTKLTANDTYAEPWAEGDEMKKLGIQPIPMMDRDKKDEVPQGQLGFYNAVAIPCYTTLTQILPPTEPLLKACRDNLNQWEKVIRGEETAMWISGPATSKSTSEKPTRKVDD

SEQ ID NO: 13 - protein, 343 aa, Rattus norvegicus
TSEEWQGLMHFNLPARICRDIELFHFDIGPFENMWPGIFVYMIHRSCGTSCFELEKLCRFIMSVKKNYRRVPYHNWKHAVTVAHCMYAILQNNNGLFTDLERKGLLIACLCHDLDHRGFSNSYLQKFDHPLAALYSTSTMEQHHFSQTVSILQLEGHNIFSTLSSSEYEQVLEIIRKAIIATDLALYFGNRKQLEEMYQTGSLNLHNQSHRDRVIGLMMTACDLCSVTKLWPVTKLTANDIYAEFWAEGDEMKKLGIQPIPMMDRDKRDEVPQGQLGFYNAVAIPCYTTLTQILPPTEPLLKACRDNLNQWEKVIRGEETAMWISGPATSKSTSEKPTRKVDD

SEQ ID NO: 14 - protein, 339 aa, Artificial Sequence
  Consensus sequence
TAEEWQGLMFNLPRICKDIELFHFDIGPFENMWPGIFVYMIHRCGTSCFELEKLCRFIMSVKKNYRRVPYHNWKHAVTVAHCMYAILQNNNGLFTDLERKGLLIACLCHDLDHRGFSNSYLQKFDHPLAALYSTSTMEQHHFSQTVSILQLEGHNIFSTLSSSEYEQVLEIIRKAIIATDLALYFGNRKQLEEMYQTGSLNLHNQSHRDRVIGLMMTACDLCSVTKLWPVTKLTANDIYAEFWAEGDEMKKLGIQPIPMMDRDKKDEVPQGQLGFYNAVAIPCYTTLTQILPPTEPLLKACRDNLNQWEKVIRGEETAMWISGPATSKSTSKPTRKVDD